(19) United States
(12) Patent Application Publication
(10) Pub. No.: US 2026/0308679 A1
(43) Pub. Date: Oct. 8, 2026

(54) PLASMONIC NANOANTENNA AND USE OF SAME IN SINGLE MOLECULE SEQUENCING

(71) Applicant: SWAN GENOMICS PTY LTD, St. Ives Chase (AU)

(72) Inventors: Lawrence LEE, St. Ives Chase (AU); Min HUANG, St. Leonards (AU); James BROWN, Lane Cove North (AU); Alexander Daniel MARS, Vaucluse (AU)

(21) Appl. No.: 19/382,135

(22) Filed: Nov. 6, 2025

Related U.S. Application Data
(63) Continuation of application No. PCT/AU2024/050452, filed on May 8, 2024.

(30) Foreign Application Priority Data
May 8, 2023 (AU) 2023901389

(51) Int. Cl. C12Q 1/6874; C12Q 1/48; C12Q 1/6876

(52) U.S. Cl. CPC C12Q 1/6874; C12Q 1/48; C12Q 1/6876; C12Y 207/07

(57) ABSTRACT

The present disclosure relates generally to methods of sequencing nucleic acid molecules, such as DNA and more specifically single molecules of DNA, using polymerase enzyme located within an area of electrical field enhancement. The present disclosure also relates to plasmonic nanoantenna which may be used in nucleic acid sequencing, methods of making said nanoantenna, arrays comprising said nanoantenna and methods of using said nanoantenna and arrays in single molecule sequencing technologies.

### CROSS-REFERENCE

This application is a continuation of International Patent Application No. PCT/AU2024/050452, filed May 8, 2024, which claims priority from Australian Provisional Patent Application No. 2023901389 filed on 8 May 2023, the content of each of which is incorporated herein by reference in their entirety.

### INCORPORATION BY REFERENCE OF SEQUENCE LISTING

The instant application contains a Sequence Listing which has been submitted electronically in XML format and is hereby incorporated by reference in its entirety. Said XML copy, created on Apr. 28, 2026, is named 67649-701_301_SL.xml and is 27,617 bytes in size.

### TECHNICAL FIELD

The present disclosure relates generally to methods of sequencing nucleic acid molecules, plasmonic nanoantenna, methods of making said nanoantenna, arrays comprising said nanoantenna and methods of using said nanoantenna and arrays in single molecule sequencing technologies.

### BACKGROUND

DNA sequencing reads the order in which the four nucleotide bases—adenine (A), thymine (T), cytosine (C) and guanine (G)—arrange into the long biopolymer that encodes the genes of all living organisms. The ability to sequence DNA has revolutionised the biological sciences and is having a rapidly expanding impact on multiple industries including medicine, agriculture, and biotechnology. The DNA sequencing market is predicted to reach $60 billion by 2030. Nonetheless, there exist serious challenges for the current wave of industry-leading high-throughput sequencing methods including the need for large quantities of DNA, limitations on the maximum DNA length that can be continuously read, and the computational resources needed to build the sequence from raw data. As such, a highly coveted goal is to accurately read the sequence of a single DNA molecule in real time.

### SUMMARY

Recently, significant progress has been made with single-molecule sequencing technologies and two emerging technologies—Oxford Nanopore's ‘current blockade’ and PacBio's ‘zero mode waveguide’ sequencing—now lead the market. Each company is already valued at ~$US 2 billion. However, both technologies use methods subject to fundamental limits in our ability to precisely manipulate a single DNA molecule and confine the observation volume to a single nucleotide. These limitations result in substantial error rates, and thus accurate sequencing still requires multiple repeated sequencing reactions to attain accuracy statistically. As such, there is a need for true single-molecule DNA sequencing.

The use of metallic nanoparticles and that plasmon may serve as auxiliary antenna for enhancing fluorescence of molecules, e.g. in the field of optical sensors. By coupling the electromagnetically irradiated molecules with the plasmon of the nanoparticles, it is possible to influence intensity and dynamic of the interaction between molecules and light, making it possible to increase the emitting rate of fluorescent molecules. As such, nanoparticle plasmon resonance-based sensing is a label-free approach that has been developed for detecting the presence of analytes in a sample and/or for studying biomolecular interactions in real time.

Provided herein are plasmon resonance-based sensing methods and compositions for use in improved sequencing of DNA molecules. The plasmon resonance-based sensing methods and compositions provided herein also encompass solutions to challenges of developing plasmon resonance-based sensing methods for sequencing, wherein such challenges were previously not contemplated or known. While prior attempts at applying plasmon resonance-based sensing to the single molecule level for complex biological molecules like proteins have been made (e.g., US20130252825A1), plasmon resonance-based sensing methods and compositions required for the effective performance of sequencing reactions have not been demonstrated experimentally and/or remains a challenge due to inter alia the difficulties with immobilising large biological molecules e.g., such as enzymes, within the hotspot formed by the nanoparticles in such a way that those biological molecules (e.g., a polymerase enzyme) retain their function. Thus, new methods and tools for analysing single molecules using nanoparticle plasmon resonance-based approaches are required. The present disclosure is based, in part, on the recognition by the inventors that, although plasmonic nanoantenna have been developed and shown to be useful for a range of applications, previous effort towards the use of plasmonic nanoantenna in biological applications have been limited by the types of molecules that can be tethered to the DNA scaffold within the ‘plasmonic hotspot’ defining the area of electrical field enhancement. The limitations of existing plasmonic nanoantenna technology stem, in part, from the fact that the relatively small size of the plasmonic hotspot that is required for meaningful enhancement of signal limits the size of the molecules that can be bound within the hotspot, as well as the size of analytes that can access the hotspot during use. Whilst more recent efforts have resulted in plasmonic nanoantenna with so called ‘cleared space’ within the plasmonic hotspot, there is no evidence to date of enzymes being bound within the plasmonic hotspot of plasmonic nanoantenna, much less evidence that an enzyme bound within the hotspot would be able to retain its activity in the face of steric constraints caused by the limited space within the DNA origami structure. However, the present inventors have now developed a plasmonic nanoantenna comprising a nanoscopic DNA scaffold onto which two plasmonic nanoparticles are immobilised to form a plasmonic hotspot therebetween and demonstrated for the first time that an active protein (i.e., DNA polymerase) can be immobilised within the unoccupied area (or ‘cleared space’) within the plasmonic hotspot. The inventors have also demonstrated experimentally that DNA polymerase retains its biological function when immobilised within the plasmonic hotspot of their nanoantenna design and that the plasmonic hotspot retains sufficient electrical field intensity to enhance fluorescence signal whilst containing the DNA polymerase. In this regard, the inventors have shown that the intensity of light emitted from fluorophores attached to or co-located with the protein molecule within the hotspot can be enhanced relative to the intensity of light emitted from the corresponding fluorophores outside the hotspot. Most significantly, however, is the experimental finding by the inventors that, when DNA polymerase is tethered within the plasmonic hotspot of their nanoantenna in the presence of fluorescently-labelled deoxynucleotide triphosphates (dNTPs) and a DNA template, it is possible to detect the order of incorporation of specific, fluorescently-labelled dNTPs into a growing strand of DNA by the DNA polymerase enzyme during DNA synthesis by detecting the enhanced and unique fluorescence signal emitted from the respective dNTPs upon their incorporation within the plasmonic hotspot. Thus, the inventors have shown that the plasmonic nanoantenna of the disclosure can be used in single molecule DNA sequencing applications.

Accordingly, in one example, the disclosure provides method of sequencing a nucleic acid analyte, comprising:
(I) contacting a nucleic acid polymerase with the nucleic acid analyte and labelled nucleotides for a time and under conditions such that the labelled nucleotides are sequentially incorporated by the polymerase into a polynucleotide having a sequence which is complementary to a polynucleotide sequence of the nucleic acid analyte, wherein the polymerase is positioned within an area of electrical field enhancement; and each labelled nucleotide comprises:
   (i) an adenine nucleotide (A), a guanosine nucleotide (G), a thymine nucleotide (T), or a cytosine nucleotide (C),
   (ii) a fluorophore, and
   (iii) a polyphosphate linker binding the nucleotide to the fluorophore;
      wherein the A, G, T and C are each independently linked to a fluorophore via the polyphosphate,
      wherein each of labelled nucleotides A, G, T and C has a distinct fluorescence emission signature when the fluorophore to which the respective nucleotide is linked is excited, and
      wherein the distinct fluorescence emission signatures of the labelled nucleotides are enhanced upon their incorporation by the nucleic acid polymerase into the sequence which is complementary to a polynucleotide sequence of the nucleic acid analyte; and
(II) detecting the order of enhanced distinct fluorescence emission signatures as the labelled nucleotides are sequentially incorporated by the nucleic acid polymerase into the polynucleotide having a sequence which is complementary to a polynucleotide sequence of the nucleic acid analyte, and thereby determining the sequence of the nucleic acid analyte by determining the order of nucleotides incorporated into the polynucleotide having a sequence which is complementary to a polynucleotide sequence of the nucleic acid analyte.

In one example, the fluorescence emission signatures of the labelled nucleotides are not enhanced outside of the area of electrical field enhancement.

In one example, the method comprises contacting the nucleic acid polymerase with the nucleic acid analyte in the presence of each of the labelled nucleotides A, G, T and C.

In some examples, the nucleotide of the labelled nucleotide may be further selected from a synthetic nucleotide. In one example, the synthetic nucleotide is selected from 5-methylcytidine, N-methylcytidine, and N6-methyladenosine.

The synthetic nucleotide is preferably linked to a fluorophore via the polyphosphate. In accordance with examples where the method employs a synthetic nucleotide linked to a fluorophore via the polyphosphate, each of the nucleotides A, G, T, and C and the synthetic nucleotide has a distinct fluorescence emission signature when the fluorophores to which the respective nucleotides are linked are excited.

In one example, the distinct enhanced fluorescence emission signatures of the labelled nucleotides are enhanced by two times or more (e.g., by about three times or more, or by about four times or more, or by about five times or more, or by about six times or more, or by about seven times or more, or by about eight times or more or by about nine times or more) as compared to the distinct fluorescence emission signatures of corresponding labelled nucleotides outside of the area of electrical field enhancement. In some examples, the enhanced distinct fluorescence emission signatures of the labelled nucleotides are enhanced by an order of magnitude or more (e.g., about 10× or more, or about 20× or more, or about 30× or more, or about 40× or more, or about 50× or more, or about 60× or more, or about 70× or more, or about 80× or more, or about 90× or more, or about 100× or more) as compared to the distinct fluorescence emission signatures of corresponding labelled nucleotides outside of the area of electrical field enhancement. In further examples, the enhanced distinct fluorescence emission signatures of the labelled nucleotides are enhanced by at least about 100× or more (e.g., at least about 150×, or at least about 200×, or at least about 250×, or at least about 300×, or at least about 350×, or at least about 400×, or at least about 450×, or at least about 500×, or at least about 550×, or at least about 600×, or at least about 650×, or at least about 700×, or at least about 750×, or at least about 800×, or at least about 850×, or at least about 900×, or at least about 950×, or at least about 1000×) as compared to the distinct fluorescence emission signatures of corresponding labelled nucleotides outside of the area of electrical field enhancement.

The distinct fluorescence emission signatures as described herein may be distinguishable from one another based on differences in peak emission wavelength, differences in fluorescence emission intensity, differences in fluorescence emission duration, differences in fluorescence lifetime, differences in duration between successive fluorescence emissions, or any combinations thereof.

In one example, each fluorophore has a peak emission wavelength independently selected from an emission wavelength in the visible spectrum, an emission wavelength in the ultraviolet (UV) spectrum, an emission wavelength in the infrared (IR) spectrum, and an emission wavelength in the near-IR spectrum. In one example, each fluorophore has a peak emission wavelength independently selected from an emission wavelength between about 350 nm and 850 nm. In some examples, one or more or each of the fluorophores has a low quantum efficiency (or low quantum yield).

In one example, the distinct fluorescence emission signatures of two or more species of the labelled nucleotides are distinguishable based on differences in peak emission wavelength. In one example, each different species of nucleotide is linked to a fluorophore having a distinct peak emission wavelength. In accordance with any example in which labelled nucleotides are distinguishable based on differences in peak emission wavelength, the peak emission wavelengths of the labelled nucleotides are separated by 10 nm or more relative to each other. For example, the peak emission wavelengths may be separated by about 25 nm or more relative to each other. For example, the peak emission wavelengths may be separated by about 50 nm or more relative to each other. For example, the peak emission wavelengths may be separated by about 75 nm or more relative to each other. For example, the peak emission wavelengths may be separated by about 100 nm or more relative to each other.

In one example, the distinct fluorescence emission signatures of two or more species of the labelled nucleotides are distinguishable based on differences in fluorescence lifetime.

Alternatively, or in addition, two or more species of nucleotides are linked to the same fluorophore and different species of nucleotides are distinguished based on differences in fluorescence emission intensity and/or fluorescence emission duration. In one example, the different species of nucleotides are distinguished based on differences in fluorescence emission intensity. In one example, the different species of nucleotides are distinguished based on differences in fluorescence emission duration.

As described herein, each labelled nucleotide used in the method of the disclosure comprises a nucleotide linked to a fluorophore via a polyphosphate. In one example, the polyphosphate is a tri-, tetra-, penta- or hexa-phosphate. For example, the polyphosphate is a triphosphate. For example, the polyphosphate is a tetra-phosphate. For example, the polyphosphate is a penta-phosphate. For example, the polyphosphate is a hexa-phosphate.

In some examples, the fluorophore is bound to the phosphate furthest from the nucleotide.

In some examples, the method comprises contacting the nucleic acid polymerase with the nucleic acid analyte and labelled nucleotides the presence of one or more quenching agents.

In one example, the method comprises providing a source of electromagnetic waves to the area of electrical field enhancement, wherein the electromagnetic waves comprise wavelengths sufficient to excite the fluorophores linked to the labelled nucleotides.

In each of the foregoing examples, the step of detecting the order of enhanced distinct fluorescence emission signatures at (II) is performed using fluorescence microscopy or a fluorometer. For example, the step of detecting the order of enhanced distinct fluorescence emission signatures at (II) is performed using total internal reflection fluorescence (TIRF) microscopy. For example, the step of detecting the order of enhanced distinct fluorescence emission signatures at (II) is performed using confocal microscopy. For example, the step of detecting the order of enhanced distinct fluorescence emission signatures at (II) is performed using epifluorescence microscopy.

In each of the foregoing examples, the nucleic acid analyte may be DNA. In one example, the DNA is genomic DNA. In another example, the DNA is complementary DNA (cDNA) which has been derived from an RNA. In accordance with examples in which the nucleic acid analyte is cDNA, the method may comprise determining the sequence of RNA based on the sequence for corresponding cDNA.

In some examples, the method further comprises contacting the DNA with an oligonucleotide primer which is capable of hybridizing specifically to a region of the DNA to initiate synthesis by the nucleic acid polymerase e.g., DNA polymerase. In some examples, the region of the DNA to which the oligonucleotide primer hybridizes is an oligonucleotide adapter, and the oligonucleotide primer is substantially complementary to the oligonucleotide adapter. In certain examples, the DNA to be sequenced comprises the oligonucleotide adapter at its 3′ end. For example, the DNA comprising the oligonucleotide adapter at its 3′ end may be single-stranded DNA. For example, the DNA comprising the oligonucleotide adapter at its 3′ end may be double-stranded DNA. In other examples, the DNA to be sequenced is a double-stranded DNA flanked by two hairpin loops, wherein the hairpin loops are formed of single-stranded DNA and at least one of the hairpin loops comprises the oligonucleotide adapter. The method may further comprise ligating the oligonucleotide adapter to the DNA to be sequenced prior to contacting step at (I).

In other examples, the DNA to be sequenced is single-stranded DNA and comprises a hairpin oligonucleotide adapter at its 3′ end, wherein the hairpin oligonucleotide adapter comprises a sequence which is internally complementary and capable of forming a hairpin comprising a duplex region to initiate synthesis by the nucleic acid polymerase e.g., DNA polymerase. The method may further comprise ligating the hairpin oligonucleotide adapter to the DNA to be sequenced prior to contacting step at (I).

In each of the foregoing examples, the nucleic acid analyte may be a single molecule. Accordingly, the method may comprise contacting the nucleic acid polymerase with a single nucleic acid molecule, e.g., a single molecule of DNA.

In each of the forgoing examples, the area of electrical field enhancement may be produced by a plasmonic hotspot, wherein the plasmonic hotspot is produced by a plasmonic nanoantenna. The plasmonic nanoantenna may comprise at least two plasmonic nanoparticles.

In one particular example, the plasmonic nanoantenna used to produce the area of electrical field enhancement in the method comprises:
two plasmonic nanoparticles;
a nanoscopic nucleic acid scaffold; and
the nucleic acid polymerase;
wherein:
(a) the two plasmonic nanoparticles are attached to the nanoscopic nucleic acid scaffold and positioned relative to one another such that a plasmonic hotspot exists between the two plasmonic nanoparticles,
(b) the plasmonic hotspot comprises a region unoccupied by the nanoscopic nucleic acid scaffold, and
(c) the nucleic acid polymerase is bound to the nanoscopic nucleic acid scaffold and is positioned within the region unoccupied by the nanoscopic nucleic acid scaffold, and
(d) optionally, a nucleic acid analyte is bound to the nanoscopic nucleic acid scaffold.

The present disclosure also provides a plasmonic nanoantenna comprising:
two plasmonic nanoparticles;
a nanoscopic nucleic acid scaffold; and
a nucleic acid polymerase;
wherein:
(a) the two plasmonic nanoparticles are bound to the nanoscopic nucleic acid scaffold and positioned relative to one another such that a plasmonic hotspot exists between the two plasmonic nanoparticles,
(b) the plasmonic hotspot comprises a region unoccupied by the nanoscopic nucleic acid scaffold, and
(c) the nucleic acid polymerase is bound to the nanoscopic nucleic acid scaffold and is positioned within the region unoccupied by the nanoscopic nucleic acid scaffold, and
(d) optionally, a nucleic acid analyte is bound to the nanoscopic nucleic acid scaffold.

In other examples of the method and/or plasmonic nanoantenna of the disclosure, the plasmonic nanoantenna may comprises more than two plasmonic nanoparticles, each bound to the nanoscopic nucleic acid scaffold and positioned relative to one another such that a plasmonic hotspot exists between the plasmonic nanoparticles. For example, the plasmonic nanoantenna may comprise three plasmonic nanoparticles. For example, the plasmonic nanoantenna may comprise four plasmonic nanoparticles. For example, the plasmonic nanoantenna may comprise five plasmonic nanoparticles. For example, the plasmonic nanoantenna may comprise six plasmonic nanoparticles. For example, the plasmonic nanoantenna may comprise seven plasmonic nanoparticles. For example, the plasmonic nanoantenna may comprise eight plasmonic nanoparticles. For example, the plasmonic nanoantenna may comprise nine plasmonic nanoparticles. For example, the plasmonic nanoantenna may comprise ten or more plasmonic nanoparticles.
a. In accordance with examples of the method and/or the plasmonic nanoantenna of the disclosure in which the plasmonic nanoantenna comprises two plasmonic nanoparticles, the region within the plasmonic hotspot which is unoccupied by the nanoscopic nucleic acid scaffold is defined by a three-dimensional space having a length (L) between the two plasmonic nanoparticles, a height (H) at a midpoint (L/2) that is perpendicular to (L), and a width (W) at L/2 that is perpendicular to L and H, wherein at L/2, W is selected from about 20 nm to about 100 nm and H is selected from about 10 nm to about 100 nm, and wherein L is measured at the shortest distance between the two plasmonic nanoparticles. In some examples, L is selected from about 20 nm to about 100 nm. In other examples, L is selected from about 20 nm to about 50 nm.

In yet other examples, the region of the plasmonic hotspot which is unoccupied by the nanoscopic nucleic acid scaffold has a volume of at least about 1 zL, such as from about 1 zL to about 10 zL, or from about 5 zL to about 10 zL. In certain examples, the region of the plasmonic hotspot which is unoccupied by the nanoscopic nucleic acid scaffold has a volume which is greater than or equal to about 10 zL. For example, the region of the plasmonic hotspot which is unoccupied by the nanoscopic nucleic acid scaffold has a volume of about 10 zL to about 50 zL. For example, the region of the plasmonic hotspot which is unoccupied by the nanoscopic nucleic acid scaffold may have a volume of about 20 zL to about 40 zL.

In accordance with the foregoing examples, the region of the plasmonic hotspot unoccupied by the nanoscopic nucleic acid scaffold is sufficiently large to accommodate the nucleic acid polymerase and to retain biological activity of the nucleic acid polymerase. In some examples, the region unoccupied by the nanoscopic nucleic acid scaffold is less than or equal to 50% of the plasmonic hotspot. However, in other examples, the region unoccupied by the nanoscopic nucleic acid scaffold is more than 50% of the plasmonic hotspot. In some examples, the region unoccupied by the nanoscopic nucleic acid scaffold is 60% or more of the plasmonic hotspot. In certain examples, the plasmonic hotspot is substantially unoccupied by the nanoscopic nucleic acid scaffold. For example, the region unoccupied by the nanoscopic nucleic acid scaffold may be 70% or more of the plasmonic hotspot. In some examples, the region unoccupied by the nanoscopic nucleic acid scaffold is 75% or more of the plasmonic hotspot. In some examples, the region unoccupied by the nanoscopic nucleic acid scaffold is 80% or more of the plasmonic hotspot. In some examples, the region unoccupied by the nanoscopic nucleic acid scaffold is 85% or more of the plasmonic hotspot. In some examples, the region unoccupied by the nanoscopic nucleic acid scaffold is 90% or more of the plasmonic hotspot.

In each of the foregoing examples, the region unoccupied by the nanoscopic nucleic acid scaffold may take different shapes or form. In one example, the region unoccupied by the nanoscopic nucleic acid scaffold may be amorphous. Alternatively, the region unoccupied by the nanoscopic nucleic acid scaffold may have a substantially defined shape. For example, the region unoccupied by the nanoscopic nucleic acid scaffold may be spherical, elliptical, cubic or cuboid.

According to some examples of the method and/or the plasmonic nanoantenna of the disclosure, the average interparticle distance between the plasmonic nanoparticles is about 10 nm to about 100 nm, wherein the interparticle distance between two particles is determined as the shortest distance between the surfaces of the respective particles. For example, the average interparticle distance between the plasmonic nanoparticles may be about 10 nm to 80 nm. For example, the average interparticle distance between the plasmonic nanoparticles may be about 10 nm to 50 nm. For example, the average interparticle distance between the plasmonic nanoparticles may be about 20 nm to 80 nm. For example, the average interparticle distance between the plasmonic nanoparticles may be about 20 nm to 50 nm. For example, the average interparticle distance between the plasmonic nanoparticles may be about 30 nm to 50 nm. For example, the average interparticle distance between the plasmonic nanoparticles may be about 20 nm to 40 nm. For example, the average interparticle distance between the plasmonic nanoparticles may be about 30 nm to 40 nm. In one particular example, the plasmonic nanoparticles have an average interparticle distance of about 30 nm.

In some examples of the method and/or plasmonic nanoantenna of the disclosure, the plasmonic nanoantenna comprises the nucleic acid analyte bound to the nanoscopic nucleic acid scaffold. For example, the nucleic acid analyte may be bound non-covalently (e.g., through hydrogen bonding) to the nanoscopic nucleic acid scaffold.

The plasmonic nanoparticles of the plasmonic nanoantenna described herein may be formed of any material which possesses plasmonic resonance properties. Suitable plasmonic nanoparticles include, but are not limited to, metallic nanoparticles, metal alloy nanoparticles, polymeric nanoparticles and derivatives and composites thereof. In one example, each of the plasmonic nanoparticles are independently selected from metallic nanoparticles. For example, each of the two or more plasmonic nanoparticle is a metallic nanoparticle independently selected from the group consisting of gold nanoparticles, silver nanoparticles, aluminium nanoparticles, copper nanoparticles, bismuth nanoparticles, nickel nanoparticles, palladium nanoparticles and platinum nanoparticles or a nanoparticle formed of an alloy of any one thereof. In some examples, the metallic nanoparticles are solid and formed wholly of the metallic substance. In other examples, the metallic nanoparticles are formed wholly of the metallic substance but have a hollow core. In other examples, the metallic nanoparticles comprise a core (e.g., a glass, polymer or composite core) which is coated in the metallic substance to form a metallic surface. In one example, the plasmonic nanoparticles are gold nanoparticles or gold alloy nanoparticles. In one example, the plasmonic nanoparticles are silver nanoparticles or silver alloy nanoparticles. In other examples, the plasmonic nanoparticles are formed of a non-metallic material or metal oxide which has been doped to increase the total carrier load of the nanoparticle and thereby increase its plasmon resonance.

The plasmonic nanoparticles of the plasmonic nanoantenna described herein may vary in their shape and geometry. Accordingly, the plasmonic nanoparticles may be selected independently from nanospheres, nanorods, nanoprisms, nanocubes, nanoshells, nanotubes, or nanostars. The plasmonic nanoparticle on the plasmonic nanoantenna may be the same shape or they may be heterogeneous. In one example, the plasmonic nanoparticles are nanospheres.

The plasmonic nanoparticles of the plasmonic nanoantenna described herein may vary in their dimensions. In one example, each of the plasmonic nanoparticles has a largest diameter of about 5 nm to about 500 nm. In one example, each of the plasmonic nanoparticles has a largest diameter of about 10 nm to about 250 nm. In one example, each of the plasmonic nanoparticles has a largest diameter of about 20 nm to about 200 nm. In one example, each of the plasmonic nanoparticles has a largest diameter of about 50 nm to about 150 nm. In one example, each of the plasmonic nanoparticles has a largest diameter of about 80 nm to about 120 nm. In one particular example, each of the plasmonic nanoparticles has a largest diameter of about 80 nm. In another particular example, each of the plasmonic nanoparticles has a largest diameter of about 100 nm. In yet another particular example, each of the plasmonic nanoparticles has a largest diameter of about 120 nm.

In some examples, the plasmonic nanoparticles have the same largest diameter. Whereas in other examples, the largest diameter differs between plasmonic nanoparticles on the same plasmonic nanoantenna. In accordance with an example in which the largest diameter differs between plasmonic nanoparticles on the same plasmonic nanoantenna, the plasmonic nanoparticles may have an average largest diameter of about 5 nm to about 500 nm. For example, the plasmonic nanoparticles on the plasmonic nanoantenna may have an average largest diameter of about 10 nm to about 250 nm. For example, the plasmonic nanoparticles on the plasmonic nanoantenna may have an average largest diameter of about 20 nm to about 200 nm. For example, the plasmonic nanoparticles on the plasmonic nanoantenna may have an average largest diameter of about 50 nm to about 150 nm. For example, the plasmonic nanoparticles on the plasmonic nanoantenna may have an average largest diameter of about 80 nm to about 120 nm. In one particular example, the plasmonic nanoparticles on the plasmonic nanoantenna may have an average largest diameter of about 80 nm. In one particular example, the plasmonic nanoparticles on the plasmonic nanoantenna may have an average largest diameter of about 100 nm. In one particular example, the plasmonic nanoparticles on the plasmonic nanoantenna may have an average largest diameter of about 120 nm.

In one particular example, the plasmonic nanoantenna described herein comprises gold or gold-coated nanoparticles (e.g., a nanosphere) having an average largest diameter of about 100 nm.

In each of the foregoing examples describing the plasmonic nanoantenna of the disclosure and/or the use of same in the method of the disclosure, each of the plasmonic nanoparticles is coated with a coating. Preferably, the coating substantially covers the surface of each of the plasmonic nanoparticles. In one example, the coating covers about 75% or more of the surface of each of the plasmonic nanoparticles. In one example, the coating covers about 80% or more of the surface of each of the plasmonic nanoparticles. In one example, the coating covers about 85% or more of the surface of each of the plasmonic nanoparticles. In one example, the coating covers about 90% or more of the surface of each of the plasmonic nanoparticles. In one example, the coating covers about 95% or more of the surface of each of the plasmonic nanoparticles.

In some examples, the coating reduces aggregation of the plasmonic nanoparticles relative to plasmonic nanoparticles without the coating. In one example, the coating reduces aggregation of the plasmonic nanoparticles by 35% or more relative to plasmonic nanoparticles without the coating. In one example, the coating reduces aggregation of the plasmonic nanoparticles by 50% or more relative to plasmonic nanoparticles without the coating. In one example, the coating reduces aggregation of the plasmonic nanoparticles by 65% or more relative to plasmonic nanoparticles without the coating.

In each of the foregoing examples describing the coating of the plasmonic nanoparticles, the coating may comprise a plurality of oligonucleotides. In some examples, each nucleotide in each oligonucleotide of the plurality of oligonucleotides is selected from pyrimidine nucleotides. In one example, each oligonucleotide of the plurality of oligonucleotides comprises about 20 nucleotides to about 50 nucleotides. In one example, each oligonucleotide of the plurality of oligonucleotides comprises about 20 nucleotides to about 30 nucleotides.

In each of the foregoing examples describing the plasmonic nanoantenna of the disclosure and/or the use of same in the method of the disclosure, the nanoscopic nucleic acid scaffold may be composed substantially of double stranded DNA. For example, the double stranded DNA may be parallel interconnected strands of double helices. In some examples, the nanoscopic nucleic acid scaffold is composed of DNA origami structures e.g., 3D DNA origami structures. In one example, the DNA origami structure comprises a M13mp18-derived scaffold strand and complementary staple strands. In certain example examples, at least a portion of the DNA origami structures are assembled to form a U-shaped or curved structure.

In one example, the nanoscopic nucleic acid scaffold comprises a first face to which one of two plasmonic nanoparticles is anchored and a second face to which the second of two plasmonic nanoparticles is anchored. The first face of the scaffold and the second face of the scaffold may be on the same or adjacent sides of the nanoscopic nucleic acid scaffold. Alternatively, the first face of the scaffold and the second face of the scaffold may be on opposite sides of the nanoscopic nucleic acid scaffold.

In accordance with examples of the method and/or the plasmonic nanoantenna of the disclosure in which the plasmonic nanoantenna comprises two plasmonic nanoparticles, one plasmonic nanoparticle is anchored to the first face of the scaffold through one or more nucleic acid linkers and the other plasmonic nanoparticle is anchored to the second face of the scaffold through one or more nucleic acid linkers,
b. wherein each nucleic acid linker is formed by an oligonucleotide coated on the surface of one of the plasmonic nanoparticles which is hybridised to a polynucleotide forming part of, or extending from, the nanoscopic nucleic acid scaffold, and
   wherein the sequences of the respective oligonucleotides and polynucleotides which are capable of hybridising are complementary or substantially complementary to one another. In accordance with this example, the nanoscopic nucleic acid scaffold comprises one or more polynucleotides extending from the first face of the scaffold, each comprising a sequence which is complementary to a sequence of one or more oligonucleotides coating one of the two plasmonic nanoparticles. Additionally, the nanoscopic nucleic acid scaffold comprises one or more polynucleotides extending from the second face of the scaffold, each comprising a sequence which is complementary to a sequence of one or more oligonucleotides coating one of the two plasmonic nanoparticles. The polynucleotides forming part of, or extending from, the nanoscopic nucleic acid scaffold, may each be a single-stranded DNA. Preferably, the polynucleotides extending from the first and second faces of the scaffold each comprise a region of sufficient length and complementarity to hybridise to the oligonucleotides coating the surface of the plasmonic nanoparticles. In one example, the polynucleotides extending from the first and second faces of the scaffold each comprise about 20 nucleotides to about 50 nucleotides. In another example, the polynucleotides extending from the first and second faces of the scaffold comprises about 20 nucleotides to about 30 nucleotides. In each of these examples, the polynucleotides extending from the first and second faces of the scaffold each comprise a region of about 10 to about 25 nucleotides in length which is capable of hybridising to a region of corresponding length within the oligonucleotides coating the surface of the plasmonic nanoparticles.

In each of the foregoing examples describing the plasmonic nanoantenna of the disclosure and/or the use of same in the method of the disclosure, the nucleic acid polymerase is bound to, or immobilized on, the nanoscopic nucleic acid scaffold through a nucleic acid linker. In one example, the nucleic acid linker is double-stranded and comprises (i) a polynucleotide which is covalently or non-covalently bound to the nucleic acid polymerase via an amino acid within the nucleic acid polymerase, and (ii) a polynucleotide forming part of, or extending from, the nanoscopic nucleic acid scaffold, wherein the polynucleotides at (i) and (ii) comprise single-stranded DNA sequences which are complementary or substantially complementary and are capable of hybridizing to one another. In one example, the polynucleotide bound to the nucleic acid polymerase comprises a peptide tag which is bound to the nucleic acid polymerase via an isopeptide bond formed between an amino acid within the nucleic acid polymerase and an amino acid within the peptide tag. In one example, the isopeptide bond is capable of being formed spontaneously. For example, the isopeptide bond may be formed between Lys and Asp, or between Lys and Asn. In some examples, the amino acid within the nucleic acid polymerase to which the nucleic acid linker is bound is located with the N-terminal domain of the nucleic acid polymerase.

Each of the polynucleotides of the nucleic acid linker which binds or immobilises the nucleic acid polymerase on the nanoscopic nucleic acid scaffold, comprises a region of sufficient length and complementarity to hybridise to one another. In one example, each polynucleotide of the nucleic acid linker comprises about 20 nucleotides to about 50 nucleotides. In another example, each polynucleotide of the nucleic acid linker comprises about 20 nucleotides to about 30 nucleotides. In each of these examples, the polynucleotides of the nucleic acid linker each comprise a region of about 10 to about 25 nucleotides in length which are complementarity and capable of hybridizing to one another.

In each of the foregoing examples describing the plasmonic nanoantenna of the disclosure and/or the use of same in the method of the disclosure, the nucleic acid polymerase may be a DNA polymerase.

In accordance with any example described herein in which the nucleic acid polymerase (e.g., DNA polymerase) is bound to, or immobilized on, on the nanoscopic nucleic acid scaffold of the plasmonic nanoantenna through a nucleic acid linker, the nucleic acid polymerase may be modified to express an amino acid sequence which is capable of binding to a peptide tag conjugated to the nucleic acid linker.

The present disclosure also provides an array comprising a plurality of the plasmonic nanoantenna as described herein.

In one example, the array comprises a solid substrate and the plurality of plasmonic nanoantenna are immobilised on the solid substrate. For example, each plasmonic nanoantenna of the array may be immobilised on the solid substrate through its nucleic acid scaffold. In some examples, the solid substrate is glass or silica. For example, the solid substrate may be glass or silica in the form of a slide or chip.

In some examples, less than 30% of the plasmonic nanoantenna in the array are clustered in aggregates of two plasmonic nanoantenna or more. For example, less than about 25% of the plasmonic nanoantenna in the array are clustered in aggregates of two plasmonic nanoantenna or more. For example, less than about 20% of the plasmonic nanoantenna in the array are clustered in aggregates of two plasmonic nanoantenna or more. For example, less than about 15% of the plasmonic nanoantenna in the array are clustered in aggregates of two plasmonic nanoantenna or more. For example, less than about 10% of the plasmonic nanoantenna in the array are clustered in aggregates of two plasmonic nanoantenna or more.

In certain examples, the array of the disclosure may be used in the method of sequencing a nucleic acid analyte as described herein. Accordingly, in any of the foregoing examples describing methods of sequencing a nucleic acid analyte, the array comprising a plurality of plasmonic nanoantenna as described herein may be employed, such as to determine the sequence of multiple nucleic acid analytes simultaneously.

It will be appreciated by persons skilled in the art that numerous variations and/or modifications may be made to the above-described embodiments, without departing from the broad general scope of the present disclosure. The present examples and embodiments are, therefore, to be considered in all respects as illustrative and not restrictive.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1 illustrates the design of a plasmonic nanoantenna DNA sequencer of the disclosure.

FIG. 2 illustrates the design of the NanoSpanno DNA Origami Scaffold of the disclosure.

FIG. 3 illustrates synthesis of DNA origami NanoSpanno scaffold described herein. (A) Agarose gels stained with RedSafe™ DNA staining solution. gel. Ladder contains a DNA molecular weight standards; M13 in lane 2 contains the single-stranded M13 phage genome (7249 nt); NS in lane 3 contains the synthesised NanoSpanno sample. (B) A typical TEM image at 38 000× magnification, containing around 20 NanoSpanno particles. (C) 2D Class Averages for the images and 3D-model showing the proposed orientation.

FIG. 4 shows visualisation of thiolated-DNA coating of gold nanoparticles. (A) shows agarose gel electrophoresis under white transmitted light (top) where colloidal gold are observable as a red band, and with a DNA-specific stain (bottom). (B) TEM micrographs of a bare gold nanoparticle (left) and a gold nanoparticle coated with thiolated DNA (right).

FIG. 5 illustrates the stability of DNA-coated gold nanoparticles. DNA-coated gold nanoparticles were suspended in 1×TAE+6 mM MgCl₂. Agarose gel is stained with a DNA-specific stain and photographs show colour of corresponding samples. Figure discloses “AAAAAAAAAAAAAAA” as SEQ ID NO: 1, “AGAGAGAGAGAGAGA” SEQ ID NO: 3, “AGAGAGAGAGAGAGAGAGAGAGAGA” SEQ ID NO: 5, “TGTGTGTGTGTGTGTGTGTGTGTGT” as SEQ ID NO: 9, “TTTTTTTTTTTTTTTTTTTTTTTTT” as SEQ ID NO: 11, “ACACACACACACACACACACACACA” as SEQ ID NO: 13, “ACACACACACACACACACACACACA” as SEQ ID NO: 15.

FIG. 6 presents an expression Vector Map for the Recombinant DNA Polymerases. The petDuet-1 vector is shown on the left with relevant features labelled. MCS-1, the site in which all three recombinant polymerases were cloned, is partially expanded on the right. The restriction sites, and components of each recombinant protein are shown in order from N to C terminus. Figure discloses “6×His” as SEQ ID NO: 27 and “MGSSHNHHHHSQDPNSS” as SEQ ID NO: 28.

FIG. 7 provides exemplary data from IMAC and SEC purification of Klenow-SpyCatcher, and SEC purification of Alexa647 labelled Klenow-SpyCatcher. (A) shows UV280 chromatogram from IMAC purification. Green line is gradient from sample pump, orange line is conductivity, providing a measure of imidazole concentration at the sample. (B) SDS PAGE of eluted fractions from IMAC purification. (C) shows UV280 chromatogram from SEC purification. (D) SDS PAGE of eluted fractions from SEC purification. (E) shows chromatogram from SEC purification of Alexa647 labelled Klenow-SpyCatcher. Blue line is the absorbance at 280 nm, purple line is the absorbance at 650 nm. (F) SDS PAGE of eluted fractions from SEC purification of Alexa647 labelled Klenow-SpyCatcher. The gel on the left was scanned for Alexa647, and the gel on the right was scanned in brightfield.

FIG. 8 illustrates the two step process used for synthesis of DNA-SpyTag peptide. Left depicts reaction with SMCC to produce maleimide-DNA. Right depicts reaction of maleimide DNA with thiol cysteine side chain on peptide to yield DNA-peptide conjugate.

FIG. 9 presents data relating to HPLC purification of maleimide DNA. (A) HPLC chromatograph at 260 nm of 5′-Maleimide-15 nt DNA; (B) HPLC chromatograph of 3′-Maleimide-15 nt DNA; (C) Absorbance Spectra and Absorbance at 260 nm (blue) and 650 nm (orange) of 5′Cy5-3′-Maleimide-15 nt DNA.

FIG. 10 presents data relating to HPLC chromatograph of peptide-DNA conjugate. (A) HPLC chromatograph at 260 nm of 5′-SpyTag-15 nt DN. (B) HPLC chromatograph at 260 nm of 3′-SpyTag-15 nt DNA. (C) Absorbance Spectra and Absorbance at 260 nm (blue) and 650 nm (orange) of 5′Cy5-3′-SpyTag-15 nt DNA.

FIG. 11 presents an SDS PAGE gel showing conjugation of SpyTag-DNA to polymerase-SpyCatcher. (A) The left image shows Alexa 647 imaging, while the right image shows brightfield after a protein-specific stain of gels. Above, 5′-SpyTag-DNA conjugates were bound to Alexa 647 labelled Klenow-SpyCatcher, while below, 3′-SpyTag-DNA conjugates were bound to Alexa 647 labelled Klenow-SpyCatcher. In each image, the left lane shows Alexa 647 labelled Klenow, and the right lane shows Alexa 647 labelled Klenow after conjugation with SpyTag-DNA. (B) The gels show 3′-SpyTag-DNA-5′Cy5 conjugates that were bound to unlabelled Klenow-SpyCatcher. The left image displays merged imaging, while the middle image shows Cy5 imaging, and the right image shows brightfield after a protein-specific stain of gels. In each image, the left lane displays Klenow-SpyTag-DNA conjugates, and the right lane displays Klenow.

FIG. 12 illustrates DNA-labelled Polymerase function (A) 15% Tris-glycine native gel for DNA polymerase (Klenow, Tag, Phi29) activity assay. (B) 15% Tris-glycine native gel for Klenow-SpyCatcher-SpyTag-DNA conjugation (left: Alexa488, right: after staining with SYBR™ Gold Nucleic Acid Gel Stain)

FIG. 13 shows the binding kinetics of Klenow (top row) and Phi29 (bottom row) binding to primed target DNA strands. Association (A,D) and dissociation (B,E) curves were fit to biphasic exponential curves (red) as described for a range of protein concentrations, as indicated. The plateau of the association curves were plotted against protein concentration (C,F) and fit to a one-site binding model (red).

FIG. 14 shows the assembly of plasmonic nanoantenna of the disclosure. (A) i: Picture showing colour of solutions; ii: photograph of agarose gel electrophoresis where red bands correspond to colloidal gold; iii: agarose gel electrophoresis image after a DNA-specific stain of gels. (B) Example pictures of well-formed dimers with EM and cryo-EM and with different sequences. (C) Example pictures of monomers and aggregates

FIG. 15 charts the yields in different configurations and with different sequences (A) 100 nm AuNPs-25AC+NS-25TG (2 binding sites); (B) 100 nm AuNP-25AC+NS-25TG (1 binding site) (C) 100 nm AuNP-25AC+NS-25TG (0 binding site); (D) 100 nm AuNPs-25TG+NS-25AC (2 binding sites); (E) 100 nm AuNP-25TG+NS-25AC (1 binding site) (F) 100 nm AuNP-25TG+NS-25AC (0 binding site).

FIG. 16 illustrates DNA polymerase bound to the cavity of the DNA origami NanoSpanno scaffold via DNA hybridisation.

FIG. 17 shows the specific co-localisation of DNA polymerase with DNA origami scaffold. 2% agarose gel for conjugation confirmation of DNA origami scaffold and Klenow and 2% agarose gel for negative control.

FIG. 18 shows quantification of co-localisation yield of DNA polymerase and DNA origami scaffold. Data are shown for Alexa488 labelled DNA origami scaffold and Alexa647 labelled Klenow. (A) shows example fluorescent image with intensity trace from encircled spot shown in (B). (C) shows the distribution of initial Alexa-647 vs initial Alexa-488 intensity. (D) are experimentally determined co-localisation yield of DNA polymerase bound at different locations at the cavity of the DNA origami scaffold as depicted by the pictures in inset. NStop corresponds to DNA origami annealed with a molar excess of Alexa647 DNA that was complementary to the staple extension on the edge of the cavity.

FIG. 19 shows quantification of fluorescence enhancement in the presence of AuNPs. (A) Exemplary photobleaching traces as indicated. (B) Photobleaching step height distributions to measure single Alexa647 fluorophore intensities in the absence (blue) and presence (red) of AuNP binding sites on DNA origami scaffold. (C) Quantification of photobleaching rate from data shown in (B). (D) Scattering distributions of 2×AuNP binding site measurements from 488 and 647 channel, with thresholds selected using gaussian fitting of 488 channel. (E) Step height distributions of particles from 2×AuNP experiment separated based on 488 scattering as in (D). (F) Photobleaching rates from data shown in €.

FIG. 20 presents the results of repeated measurements of fluorophore intensity within hotspot region by sequential binding and unbinding of a short Alexa647 labelled DNA strand (imager strand) in absence (top) or presence (bottom) of 2AuNPs.

FIG. 21 presents the result of repeated intensity measurements of plasmonic nanoantenna in absence (orange) and presence (blue) of 1 nM imager strand.

FIG. 22 shows the amplification of single fluorescent DNA polymerase bound in the hotspot.

FIG. 23 shows polarisation dependent variation in scattering intensity of perpendicular plasmonic nanoantenna orientations. (A) i. Simulation of TIRF background intensity at increasing polarisation angles of incident light. ii. Experimentally measured background intensities at increasing polarisation angles. (B) i. Predicted scattering cross-section of dimeric AuNPs aligned along (black) or perpendicular to (blue) the axis of TIRF field propagation (black). ii. Measured intensities of particles at increasing polarisation angles enabling particle number and orientation to be inferred. Dimeric AuNPs aligned along (black) or perpendicular to (blue) the axis of TIRF field propagation, or monomeric AuNPs (green).

FIG. 24 presents traces of individual plasmonic nanoantennas in absence of imager strand after incubation with biotin-DNA complementary to AuNP bound DNA. Significant decrease in fluctuations as compared to FIGS. 21 and 22.

FIG. 25 illustrates the simulated enhancement of Alexa647 fluorophore located within the hotspot at varying orientations of plasmonic nanoantenna relative to the incident light polarisation.

FIG. 26 shows the fluorescent labelling of free dNTP molecules, including HPLC chromatograms from purification of fluorescently labelled dNTPs.

FIG. 27 present the configuration of a plasmonic nanoantenna DNA sequencer of the disclosure for single-molecule DNA sequencing reactions as described.

FIG. 28 presents example traces from 2-colour DNA sequencing experiment. Traces are from two independent plasmonic nanoantenna DNA sequencer molecules. Sequencing reactions were performed with Alexa647 labelled dGTP, and Alexa561 labelled dATP, with dCTP and dTTP nucleotides left unlabelled. Bases identified from intensity values greater than three standard deviations above background are indicated in the traces.

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

### Plasmonic Nanoantenna

The present disclosure provides a plasmonic nanoantenna comprising:
(i) two plasmonic nanoparticles;
(ii) a nanoscopic nucleic acid scaffold; and
(iii) a nucleic acid polymerase;
wherein:
(a) the two plasmonic nanoparticles are bound to the nanoscopic nucleic acid scaffold and positioned relative to one another such that a plasmonic hotspot exists between the plasmonic nanoparticles,
(b) the plasmonic hotspot comprises a region unoccupied by the nanoscopic nucleic acid scaffold,
(c) the nucleic acid polymerase is bound to the nanoscopic nucleic acid scaffold and is positioned within the region (of the plasmonic hotspot) runoccupied by the nanoscopic nucleic acid scaffold, and
optionally, a nucleic acid analyte bound to the nanoscopic nucleic acid scaffold.

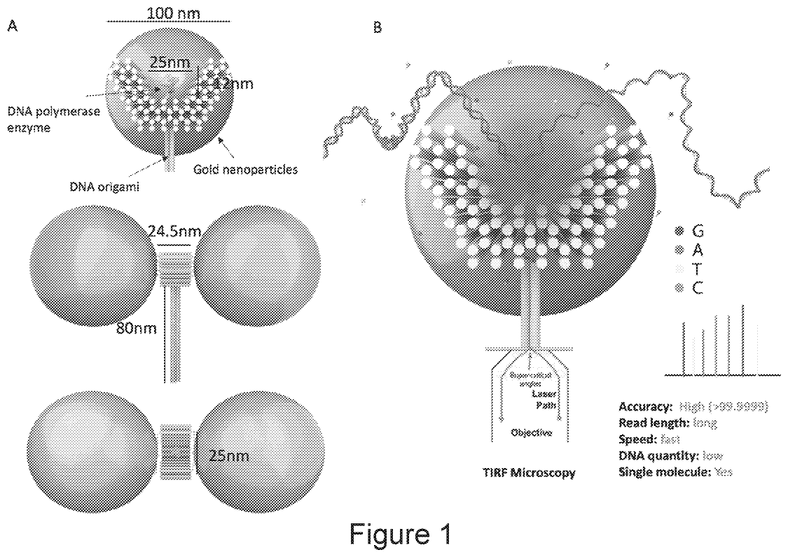

A plasmonic nanoantenna according to one exemplary embodiment of the present disclosure is illustrated in FIG. 1.

As used herein, the term “plasmonic nanoparticle” refers to a particle having nanosized dimensions whose electron density can couple with electromagnetic radiation of wavelengths that are far larger than the particle due to the nature of the dielectric-metal interface between the medium and the particles. It will be appreciated that plasmonic nanoparticles are capable of exhibit differing scattering, absorbance, and coupling properties based on their geometries and relative positions, and different geometries are contemplated herein. The nanoparticles of the disclosure may be formed of any material which possesses plasmonic resonance properties. Suitable nanoparticles include, but are not limited to, metallic nanoparticles, metal alloy nanoparticles, polymeric nanoparticles and derivatives and composites thereof. In one particular example, each of the plasmonic nanoparticles are independently selected from metallic nanoparticles.

As used herein, the term “metallic nanoparticle” shall be understood to mean a particle having nanosized dimensions and formed of a metal substance. It will be appreciated the nanoparticles may be formed in any suitable or desired shape and size, examples of which are described herein. The metallic nanoparticles may be formed wholly, or in part, of any metallic material which possesses plasmonic resonance properties. The metallic nanoparticles of the plasmonic nanoantenna described herein may be formed, either wholly or in part, of any metallic material known in the art which possesses plasmonic resonance properties. Metallic materials known to possess plasmonic resonance properties include noble metals and non-noble metals. Suitable examples include, but are not limited to, gold, silver, aluminium, copper, bismuth, nickel, palladium and platinum nanoparticles or an alloy of any one thereof. For example, one or both or each of the metallic nanoparticles may be a gold nanoparticle, a gold alloy nanoparticle or a nanoparticle having a gold or gold alloy coating. For example, one or both or each of the nanoparticles may be a silver nanoparticle, a silver alloy nanoparticle, or a nanoparticle having a silver or silver alloy coating. For example, one or both or each of the nanoparticles may be an aluminium nanoparticle, an aluminium alloy nanoparticle or a nanoparticle having an aluminium or aluminium alloy coating. For example, one or both or each of the nanoparticles may be a copper nanoparticle, a copper alloy nanoparticle or a nanoparticle having a copper or copper alloy coating. For example, one or both or each of the nanoparticles may be a bismuth nanoparticle, a bismuth alloy nanoparticle or a nanoparticle having a bismuth or bismuth alloy coating. For example, one or both or each of the nanoparticles may be a nickel nanoparticle, a nickel alloy nanoparticle or a nanoparticle having a nickel or nickel alloy coating. For example, one or both or each of the nanoparticles may be a palladium nanoparticle, a palladium alloy nanoparticle, or a nanoparticle having a palladium or palladium alloy coating. For example, one or both or each of the nanoparticles may be a platinum nanoparticle, a platinum alloy nanoparticle, or a nanoparticle having a platinum or platinum alloy coating.

In accordance with examples in which the plasmonic nanoparticles comprise a core which is coated in a metallic substance which possesses plasmonic resonance properties as described herein, the core may be formed of glass, polymer or a composite core or other suitable material.

Nanoparticles formed of non-metallic materials and metal-oxides are also contemplated for use as plasmonic nanoparticles. For example, one or more or each of the plasmonic nanoparticles may be formed of a non-metallic material or a metal oxide doped to possess or enhance plasmon resonance properties. Exemplary non-metal materials include, but are not limited to, metal oxides, chalcogenides, phosphides, nitrides and silicon. Doped plasmonic nanoparticles and methods of producing same are described in the literature and are known to the skilled person, including, but not limited to, photodoping, chemical doping, hierarchical doping and combinations thereof.

Accordingly, in one example, one or more of the plasmonic nanoparticles is formed of non-metallic material or metal oxide which has been photodoped. Methods of photodoping nanoparticles to impart or enhance plasmon resonance properties which may be employed herein are described in Petrini et al., (2023) J. Phys. Chem. C. Nanomater Interfaces, 127(3):1576-1587. In another example, one or more of the plasmonic nanoparticles is formed of non-metallic material or a metal oxide which has been doped hierarchically. Methods of hierarchically doping nanoparticles to impart or enhance plasmon resonance properties which may be employed herein are described in Kim et al., (2023) Nano Letters, 23(16):7633-7641 and Russo et al. (2018) Materials & Designs, 156:311-319. In another example, one or more of the plasmonic nanoparticles is formed of non-metallic material or metal oxide which has been chemically doped e.g., by introducing metal atoms into host lattice or structure. Examples of materials which may be chemically doped to increase the carrier density and plasmon resonance properties including metal oxides, chalcogenides, phosphides, nitrides and silicon. Methods of chemically doping nanoparticles to impart or enhance plasmon resonance properties which may be employed herein are described in Chowdhury et al., (2017) Nanoscale, 9:15591-15597 and Liu et al., (2019) Nature Communications, 10: 1394.

In some examples, the plasmonic nanoparticles are formed of or comprise the same material. For example, the plasmonic nanoparticles may each be metallic plasmonic nanoparticles as described herein. For example, the plasmonic nanoparticles may each be gold nanoparticles or nanoparticles which are coated in gold such that they have a gold surface. For example, the plasmonic nanoparticles may each be silver nanoparticles or nanoparticles which are coated in silver such that they have a silver surface.

In other examples, the plasmonic nanoparticles are formed of or comprise different materials to one another. For example, one nanoparticle may be a gold nanoparticle or a nanoparticle coated in gold, and one or more other nanoparticles may be formed of or coated in another material (e.g., another noble metal (such as silver) or other metal with plasmonic resonance properties as described herein).

The plasmonic nanoparticles described herein may be formed in any suitable or desired shape and size, for example, as a nanosphere, nanorod, nanoprism, nanocube, nanoshell, nanotube, or nanostar. It is to be understood that these are only representative examples of nanoparticles shapes and are in no way limiting on the disclosure. In one example, one or more or each of the plasmonic nanoparticles are nanospheres. In one example, one or more or each of the plasmonic nanoparticles are nanorods. In one example, one or more or each of the plasmonic nanoparticles are nanoprisms. In one example, one or more or each of the plasmonic nanoparticles are nanocubes. In one example, one or more or each of the plasmonic nanoparticles are nanoshells. In one example, one or more or each of the plasmonic nanoparticles are nanotubes. In one example, one or more or each of the plasmonic nanoparticles are nanostars. The plasmonic nanoparticle on the plasmonic nanoantenna may be the same shape or they may be heterogeneous.

In addition to shape, a skilled person will appreciate that the size of the nanoparticles in the plasmonic nanoantenna may vary. In some examples, the plasmonic nanoparticles may have the same, or substantially the same, largest diameter. For example, each of the plasmonic nanoparticles may have a largest diameter in the range of about 5 nm to about 500 nm (e.g., in the range of about 10 nm to about 250 nm, or in the range of about 20 nm to about 200 nm, or in the range of about 50 nm to about 150 nm, or in the range of about 80 nm to about 120 nm). In some examples, each of the plasmonic nanoparticles has a largest diameter of about 80 nm. In some examples, each of the plasmonic nanoparticles has a largest diameter of about 100 nm. In some examples, each of the plasmonic nanoparticles has a largest diameter of about 120 nm.

Whereas in other examples, the largest diameter may differ between plasmonic nanoparticles on the same plasmonic nanoantenna. In accordance with an example in which the largest diameter differs between plasmonic nanoparticles on the same plasmonic nanoantenna, the plasmonic nanoparticles may have an average largest diameter in the range of about 5 nm to about 500 nm. For example, the plasmonic nanoparticles on the plasmonic nanoantenna may have an average largest diameter in the range of about 10 nm to about 250 nm (e.g., in the range of about 20 nm to about 200 nm, or about 30 nm to about 200 nm, or about 40 nm to about 200 nm, or about 50 nm to about 200 nm, or about 60 nm to about 200 nm, or about 70 nm to about 200 nm, or about 80 nm to about 200 nm, or about 90 nm to about 200 nm, or about 100 nm to about 200 nm, or about 50 nm to about 150 nm, or about 75 nm to about 150 nm, or about 100 nm to about 120 nm, or about 100 nm to about 150 nm) In one example, the average diameter of the nanoparticles is about 100 nm. In one particular example, the plasmonic nanoparticles are gold or gold-coated nanoparticles (e.g., nanospheres) having an average diameter of about 100 nm. In another example, the plasmonic nanoparticles are silver or silver-coated nanoparticles (e.g., nanospheres) having an average diameter of about 100 nm.

As described herein, the plasmonic nanoantenna of the disclosure comprises two plasmonic nanoparticles, each attached to the nanoscopic nucleic acid scaffold and positioned relative to one another such that a plasmonic hotspot exists between the plasmonic nanoparticles. However, in some examples, the plasmonic nanoantenna may comprise more than two plasmonic nanoparticles (e.g., 3, or 4, or 5, or 6, or 7, or 8, or 9, or 10 or more nanoparticles), each attached to the nanoscopic nucleic acid scaffold and positioned relative to one another such that a plasmonic hotspot exists between the plasmonic nanoparticles collectively. In accordance with examples in which the plasmonic nanoantenna may comprises more than two plasmonic nanoparticles, two or more of the plasmonic nanoparticles may be aggregated.

The terms “plasmonic hotspot”, “plasmon enhancement hotspot” or similar are used herein to define an area or region between the two or more plasmonic nanoparticles where the electrical field is enhanced relative to an electrical field outside the area or region defined between the two or more plasmonic nanoparticles. In one example, a “plasmonic hotspot” is an area of electrical field enhancement due to plasmon resonance i.e., polarization of the plasmonic nanoparticles, induced by the incoming oscillating electric field (e.g. light).

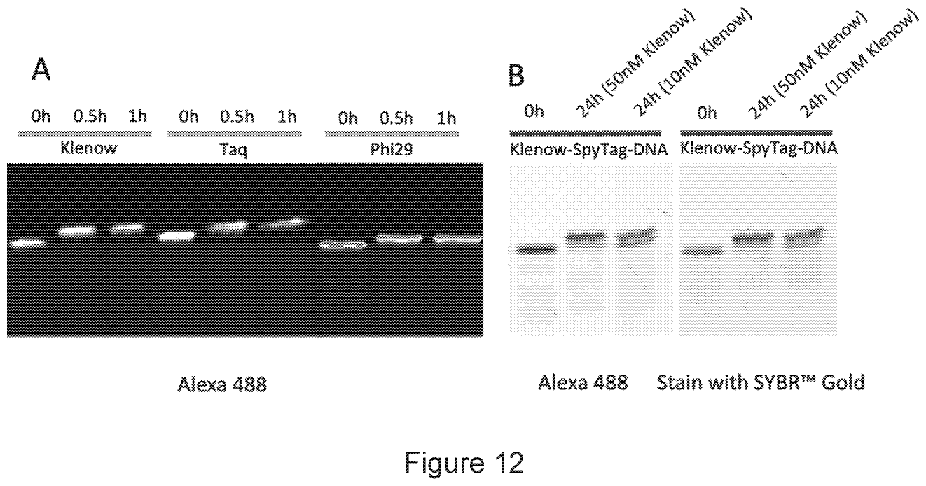

The plasmonic nanoparticles are attached to the nanoscopic nucleic acid scaffold and positioned relative to one another such that a plasmonic hotspot comprising a region unoccupied by the nanoscopic nucleic acid scaffold exists between the plasmonic nanoparticles. As used herein, the phrase “region unoccupied by nanoscopic nucleic acid scaffold”, or similar, shall be understood to refer to the region or space within the plasmonic hotspot which is accessible to the nucleic acid polymerase e.g., when bound to the nanoscopic nucleic acid scaffold. This unoccupied region may also be referred to as “cleared space” within the plasmonic hotspot or a “cleared hotspot”. Preferably, the “region unoccupied by nanoscopic nucleic acid scaffold” is sufficiently large that, when a nucleic acid polymerase is positioned therein, the enzymatic activity of the polymerase is retained. Said another way, the enzymatic activity of the polymerase is not lost due to steric effects (e.g., blockade) of the nanoscopic nucleic acid scaffold. Methods of measuring and/or determining the dimensions and/or volume of a plasmonic hotspot between at least two plasmonic particles and cleared space which is accessible to analytes are known in the art, for example, as described in Close et al., (2022) Advanced Materials Interfaces, 9(24), with specific reference to Supplementary FIG. 12 (S12). For example, in the case of a plasmonic nanoantenna described herein where the cleared space takes the form of a cube with the dimensions L=W=H=30 nm, the cleared volume of the hotspot may be calculated as 2.7e4 nm\hat{ }3=2.7e-20 L=~27 zL, wherein 1 nm\hat{ }3 is equal to 1e-24 L and 1 zL is equal to 1e-21 L. In the case of a plasmonic nanoantenna described herein where the cleared space takes the form of a sphere with a radius (r) of 15 nm, the cleared volume may be calculated as 4/3πr\hat{ }3=~14 zL, wherein 1 nm\hat{ }3 is equal to 1e-24 L and 1 zL is equal to 1e-21 L. The skilled person would also appreciate that the dimension(s) and/or volume of the region unoccupied by the nanoscopic nucleic acid scaffold can be determined based on the volume of the nanoscopic nucleic acid scaffold that overlaps with the volume and/or dimensions of the plasmonic hotspot. A skilled person may also take the following factored into account when designing a plasmonic nanoparticle having a hotspot of sufficient size and intensity, including the dimensions and design of the nanoscopic nucleic acid scaffold, wavelength of incident light polarisation of incident light, propagation of incident light, material of nanoparticles, shape of nanoparticles, volume of nanoparticles, interparticle distance, number of nanoparticles, relative position of nanoparticles, dielectric constant of surrounding medium and/or wavelength(s) of emission detection

In some examples, the region within the plasmonic hotspot which is unoccupied by the nanoscopic nucleic acid scaffold is defined by a three-dimensional space having a length (L) between the two plasmonic nanoparticles, a height (H) at a midpoint (L/2) that is perpendicular to (L), and a width (W) at L/2 that is perpendicular to L and H, wherein at L/2, W is selected from about 20 nm to about 100 nm (e.g., selected from about 30 nm to about 100 nm, or about 40 nm to about 100 nm or about 50 nm to about 100 nm, or about 20 nm to about 50 nm or about 30 nm to about 50 nm) and H is selected from about 10 nm to about 100 nm (e.g., selected from about 30 nm to about 100 nm, or about 40 nm to about 100 nm or about 50 nm to about 100 nm, or about 20 nm to about 50 nm or about 30 nm to about 50 nm), and wherein L is measured at the shortest distance between the two plasmonic nanoparticles. The skilled person will appreciate that the shape of the nanoparticle (examples of which are described herein), and the geometry of the nucleic acid scaffold, can impact the height and width of the plasmonic hotspot, as well as the height (H) and width (W) of the region within the plasmonic hotspot which is unoccupied. The skilled person will appreciate that the interparticle distance of the plasmonic nanoparticles when attached to the plasmonic nanoantenna may be varied (or “tuned”) to alter the size and magnitude of the plasmonic hotspot, as well as the region unoccupied by the nanoscopic nucleic acid scaffold. As used herein, the term “interparticle distance” refers to the distance between any two plasmonic nanoparticles on the nanoscopic DNA scaffold of the plasmonic nanoantenna In some examples, the interparticle distance between the plasmonic nanoparticles L is about 10 nm to about 100 nm (e.g., selected from about 10 nm to about 80 nm, or about 20 nm to about 80 nm, or about 30 nm to about 80 nm, or about 40 nm to about 80 nm, or about 50 nm to about 80 nm). In another example, the interparticle distance between the plasmonic nanoparticles L is about 20 nm to about 100 nm (e.g., selected from about 20 nm to about 50 nm, or about 30 nm to about 50 nm, or about 20 nm to about 40 nm, or about 30 nm to about 40 nm). In one example, the plasmonic nanoparticles have an interparticle distance L of about 20 nm to about 50 nm. In one particular example, the plasmonic nanoparticles have an interparticle distance L of about 30 nm. In certain examples, the region of the plasmonic hotspot which is unoccupied by the nanoscopic nucleic acid scaffold has a volume of at least about 1 zL, such as from about 1 zL to about 10 zL, or from about 5 zL to about 10 zL. In certain examples, the region of the plasmonic hotspot which is unoccupied by the nanoscopic nucleic acid scaffold has a volume which is greater than or equal to about 10 zL. In some examples, the region of the plasmonic hotspot which is unoccupied by the nanoscopic nucleic acid scaffold has a volume of at least about 10 zL (e.g., at least about 15 zL, or at least about 20 zL, or at least about 25 zL, or at least about 30 zL, or at least about 35 zL, or at least about 40 zL, or at least about 50 zL). For example, the region of the plasmonic hotspot which is unoccupied by the nanoscopic nucleic acid scaffold may have a volume of about 10 zL to about 50 zL (e.g., selected from about 10 zL to about 50 zL, or from about 20 zL to about 50 zL, or from about 30 zL to about 50 zL, or from about 40 zL to about 50 zL, or from about 10 zL to about 40 zL, or from about 20 zL to about 40 zL, or from about 30 zL to about 40 zL, or from about 10 zL to about 30 zL, or from about 20 zL to about 30 zL, or from about 40 zL to about 50 zL). In one example, the region of the plasmonic hotspot which is unoccupied by the nanoscopic nucleic acid scaffold as a volume of about 20 zL to about 40 zL.

Alternatively, or in addition, the region of the plasmonic hotspot unoccupied by the nanoscopic nucleic acid scaffold may be expressed as a relative percentage of the total volume of the plasmonic hotspot. In some examples, the region unoccupied by the nanoscopic nucleic acid scaffold is 70% or more (e.g., 75%, or 80%, or 85% or 90% or 95% or more) of the plasmonic hotspot.

The skilled person will appreciate that the plasmonic hotspot, as well as the region of the plasmonic hotspot unoccupied by the nanoscopic nucleic acid scaffold, may take different shapes or form e.g., depending on the choice of nanoparticles and geometry of the nucleic acid scaffold. In one example, the region unoccupied by the nanoscopic nucleic acid scaffold may be amorphous. Alternatively, the region unoccupied by the nanoscopic nucleic acid scaffold may have a substantially defined shape. For example, the region unoccupied by the nanoscopic nucleic acid scaffold may be spherical, elliptical, cubic or cuboid.

The plasmonic nanoparticles may be immobilised at predetermined positions on the surface of the nanoscopic nucleic acid scaffold via hybridisation between (i) DNA (i.e., oligonucleotides) coating the plasmonic nanoparticles, and (ii) DNA (i.e., polynucleotides) located at predetermined positions and extending from the surface of the nanoscopic nucleic acid scaffold which are complementary or substantially complementary to the DNA coating the plasmonic nanoparticles (i.e., the nanoparticles may be immobilised covalently via Watson-crick base-paring). In this way, the interparticle distance of the plasmonic nanoparticles on the surface of the nanoscopic nucleic acid can be controlled by appropriate design and positioning of the polynucleotides extending from the surface of nanoscopic nucleic acid scaffold.

In some examples, the plasmonic nanoparticles may be coated with, or conjugated/linked to, a plurality of oligonucleotides capable of hybridising to polynucleotides extending from the surface of the nanoscopic nucleic acid scaffold. For example, each plasmonic nanoparticle may be coated with a plurality of DNA oligonucleotides selected from 2 to about 100 oligonucleotides (e.g., from 2 to about 75 oligonucleotides, or from 2 to about 50 oligonucleotides, or from 2 to about 25 oligonucleotides, or from 2 to about 10 oligonucleotides). In other examples, each plasmonic nanoparticle may be coated with 5 or more DNA oligonucleotides (e.g., 6, or 7, or 8, or 9 or 10 or 11, or 12, or 13, or 14, or 15 or more DNA oligonucleotides. Preferably, the DNA oligonucleotide coating substantially covers the surface of each of the plasmonic nanoparticles. For example, the DNA oligonucleotide coating may cover about 75% or more (e.g., selected from about 80%, or about 85% or about 90% or about 95% or more) of the surface of each of the plasmonic nanoparticles.

In addition to facilitating immobilization of the coated plasmonic nanoparticles to the surface of the nanoscopic nucleic acid scaffold, the DNA oligonucleotides coated on the plasmonic nanoparticles reduces aggregation of the plasmonic nanoparticles relative to plasmonic nanoparticles without the coating during preparation of the plasmonic nanoantenna. Exemplary methods of measuring aggregation of nanoparticles are known in the art, including but not limited to, UV-visible spectroscopy, differential centrifugal sedimentation, particle counters and/or a dynamic light-scattering, particle tracking analysis and single-particle inductively coupled plasma mass spectrometry. These exemplary methods are described in Minelli et al., (2019) Langmuir, 35(14):4927-4935. In one example, the coating of DNA oligonucleotides reduces aggregation of the plasmonic nanoparticles by 35% or more (e.g., 40%, or 45%, or more) relative to plasmonic nanoparticles without the coating. In one example, the coating of DNA oligonucleotides reduce aggregation of the plasmonic nanoparticles by 50% or more (e.g., 55%, or 60%, or more) relative to plasmonic nanoparticles without the coating. In one example, the coating of DNA oligonucleotides reduce aggregation of the plasmonic nanoparticles by 65% or more (e.g., 70%, or 75%, or more) relative to plasmonic nanoparticles without the coating.

The coating or conjugation of the one or more DNA molecules (e.g., oligonucleotides) to the plasmonic nanoparticles may be performed by any method known in the art. However, in one example, the one or more oligonucleotides is bound to the plasmonic nanoparticles by a linker. In one particular example, the oligonucleotides may be thiolated in accordance with the Examples disclosed herein. However, it will be appreciated that other functional anchor groups, such as —COOH, —NH2, and —OH, may be used to covalently attach DNA oligonucleotides to the surface of nanoparticles.

The DNA oligonucleotides coating the plasmonic nanoparticles are designed such that their nucleotide sequences are complementary or substantially complementary to sequences of the cognate DNA polynucleotides extending from the surface of the nanoscopic nucleic acid scaffold and enable hybridisation therebetween. In certain examples, more than 50% of the nucleotides (e.g., more than 50%, or more than 60%, or more than 70%, or more than 80% of the nucleotides) in the oligonucleotide or plurality of oligonucleotides coating the plasmonic nanoparticles is selected from pyrimidine nucleotides (i.e., thymine and cytosine). In some examples, each nucleotide in the oligonucleotide or plurality of oligonucleotides coating the plasmonic nanoparticles is selected from pyrimidine nucleotides (i.e., thymine and cytosine).

The DNA oligonucleotide attached to the plasmonic nanoparticles, and the complementary or substantially complementary DNA polynucleotide within, or extending from, the nanoscopic nucleic acid scaffold, may be modified or unmodified. Suitable modifications are known in the art. They include for example Azo-modifications known in the art which can be used to realize photoresponsive and reversible binding and unbinding of the respective DNA molecules. Further modifications include modifications that modulate the strength of the interaction between the respective DNA molecules, such as backbone modifications that alter the surface charge (e.g. peptide nucleic acids (PNA)) or structural flexibility to induce hybridization efficiency (e.g. locked nucleic acids (LNA)). Even further modifications include the use of photocleavable structures known in the art.

As described herein, the plasmonic nanoantenna comprises a nanoscopic nucleic acid scaffold onto which the plasmonic nanoparticles and the nucleic acid polymerase molecule are immobilised or bound. The nanoscopic nucleic acid scaffold may be a nanoscopic DNA scaffold. The nanoscopic DNA scaffold may comprise or be formed of one or more DNA origami structures. The term “DNA origami structures” and similar shall be understood to refer to scaffolds or structures formed by DNA molecules which are self-assembling and produced by the so-called DNA-origami technique. Scaffolded DNA origami is a technique which was introduced in 2006 by Paul Rothemund, building on the earlier approach of structural DNA nanotechnology, and provided access to finite DNA nanoobjects. In this technique, single-stranded circular DNA molecules of typically 7.25 kb size are folded by the aid of staple strands into an array of helices through an arrangement of periodic crossovers. Extension of this concept to assemble 3D DNA structures has led to the establishment of a robust and reliable method to fabricate DNA nanostructures (or “nanoscopic DNA scaffolds”) with dimensions in the 20 to 100 nm range. However, larger structures are possible. DNA origami structures can be a bundle of tubes or pipes, e.g. 3-, 6-, or 12-helix bundles, or can have a substantially flat, rectangular shape. In this way, the DNA origami structures of varying size and dimensions can be combined (e.g., stacked or layered) to construct a nanoscopic DNA scaffold of any particular shape. Origami structures can be used as “molecular pegboards” with an addressable surface area of a few thousand nm² for arranging arbitrary objects-of-interest (OOI) with a “single pixel” resolution of about 6 nanometers. Furthermore, the shape, dimensions and contour of the surface of origami structures may be tailored such that the nanoscopic DNA scaffold has a surface which is geometrically suitable for attaching molecules of interest (e.g., the plasmonic nanoparticles and polymerase as described herein. Methods for generating DNA origami structures, including those having DNA sequence strands protruding from the structure, are known in the art. In one example, nanoscopic DNA scaffold comprises one or more 3D DNA origami structures. In certain example examples, at least a portion of the DNA origami structures are assembled to form a U-shaped or curved structure. Exemplary U-shaped structures are illustrated in FIGS. 1-3.

In accordance with an example in which the nanoscopic DNA scaffold is assembled in a U-shape, the nanoscopic DNA scaffold will comprise a first face to which one of two plasmonic nanoparticles is anchored via a nucleic acid linker and a second face to which a second of two plasmonic nanoparticles is anchored via a nucleic acid linker. The first face of the scaffold and the second face of the scaffold may be on the same or adjacent sides, or on opposite sides, of the nanoscopic DNA scaffold.

In one example, the nanoscopic DNA scaffold will comprise (i) one or more DNA polynucleotides extending from the first face of the scaffold, each comprising a sequence which is complementary to a sequence of one or more DNA oligonucleotides coating one of the two plasmonic nanoparticles, and (ii) one or more DNA polynucleotides extending from the second face of the scaffold, each comprising a sequence which is complementary to a sequence of one or more DNA oligonucleotides coating one of the two plasmonic nanoparticles. The DNA polynucleotides forming part of, or extending from, the nanoscopic DNA scaffold, may each be or comprise a single-stranded DNA. The DNA polynucleotides extending from the first and second faces of the scaffold will each comprise a region of single-stranded DNA which is of sufficient length and complementarity to the DNA oligonucleotides coating the plasmonic nanoparticles to permit hybridisation. Upon hybridisation of a cognate DNA polynucleotide and DNA oligonucleotides to form a nucleic acid linker, a plasmonic nanoparticle comprising to DNA oligonucleotide is anchored to the surface of the nanoscopic DNA scaffold. Each nucleic acid linker anchoring a plasmonic nanoparticle to the nanoscopic DNA scaffold will be formed by one of the DNA oligonucleotides coated on the surface of the plasmonic nanoparticles hybridised to a DNA polynucleotide forming part of, or extending from, the nanoscopic DNA scaffold. In one example, the DNA polynucleotides extending from the first and second faces of the scaffold comprise a M13mp18-derived scaffold sequence and the DNA oligonucleotides coating the plasmonic nanoparticles comprises a staple sequence which is complementary thereto.

The length of the DNA polynucleotides extending from the first and second faces of the scaffold may be varied as desired. However, in certain examples, the DNA polynucleotides extending from the first and second faces of the scaffold each comprise about 20 nucleotides to about 50 nucleotides (e.g., from about 20 nucleotides to about 40 nucleotides, or about 20 nucleotides to about 30 nucleotides, or about 30 nucleotides to about 50 nucleotides, or about 30 nucleotides to about 50 nucleotides). In one example, the DNA polynucleotides extending from the first and second faces of the scaffold comprise about 20 nucleotides to about 30 nucleotides.

In certain examples, each plasmonic nanoparticles is anchored to the nanoscopic DNA scaffold via multiple nucleic acid linkers (e.g., via 2, or 3, or 4, or 5, or 6, or 7, or 8, or 9, or 10 or more nucleic acid linkers). It will be appreciated that the number of nucleic acid linkers connecting each plasmonic nanoparticle positively correlates with the binding strength of the plasmonic nanoparticle to the nanoscopic DNA scaffold. Accordingly, the number of DNA polynucleotides extending from the nanoscopic DNA scaffold and which hybridise to DNA oligonucleotides coated on the plasmonic nanoparticles, can be chosen freely to accomplish a desired binding strength and/or a desired hybridization efficiency depending from the particular application of interest. Furthermore, the length of the region of Watson-Crick pairing in the nucleic acid linker (i.e., between the DNA polynucleotide(s) extending from the nanoscopic DNA scaffold and the DNA oligonucleotide(s) attached to the plasmonic nanoparticles) can be varied to accomplish a desired binding specificity and/or binding strength and/or a desired hybridization efficiency, depending from the particular application of interest. In certain examples, the region of Watson-Crick pairing is from about 8-30 nucleotides in length, or between about 10-25 nucleotides in length, or about 12-20 nucleotides in length. The terms “hybridise”, “hybridising” or similar as used herein refers to a process whereby two complementary or substantially complementary nucleic acid sequences anneal to each in accordance with Watson-Crick base pairing rules.

The skilled person will appreciate that the DNA oligonucleotides attached to the plasmonic nanoparticles, and the complementary or substantially complementary DNA polynucleotide within, or extending from, the nanoscopic DNA scaffold (which together hybridise to form the nucleic acid linkers that anchor the plasmonic nanoparticles to the nanoscopic DNA scaffold), may be of any length. For example, the respective DNA molecules may have lengths independently selected from between about 20 to about 50 nucleotides in length or between about 20 to about 40 nucleotides in length, or between about 20 to about 30 nucleotides in length. Preferably, the region of Watson-Crick pairing between the DNA polynucleotide sequence(s) extending from the nanoscopic nucleic acid scaffold (e.g., DNA origami structures) and the DNA oligonucleotide sequences attached to the plasmonic nanoparticles is from about 8-30 nucleotides in length, or between about 10-25 nucleotides in length, or about 12-20 nucleotides in length. e.g.

The terms “hybridise”, “hybridising” or similar as used herein refers to a process whereby two complementary or substantially complementary nucleic acid sequences anneal to each in accordance with Watson-Crick base pairing rules.

As used herein, the term “substantially complementary” refers to two sequences that are fully complementary or that are partially complementary to one another, such that they will hybridize under appropriately stringent hybridization conditions. Said another way, the term “substantially complementary” is used to indicate a sufficient degree of complementarity or precise pairing such that stable and specific binding occurs between two nucleic acid sequences e.g., between the DNA sequences attached to the plasmonic nanoparticles, and the complementary or substantially complementary DNA sequences within the nanoscopic DNA scaffold. It is understood that the sequence of a nucleic acid need not be 100% complementary to that of its target or complement (although this may be one preferred example). For example, the sequence of a nucleic acid may be at least 80%, or at least 85%, or at least 90%, or at least 95%, or at least 96%, or at least 97%, or at least 98%, or at least 99% complementary to the sequence of its target or complement.

As described herein, the present disclosure is based, in part, on the ability to immobilise a nucleic acid polymerase within the unoccupied area (or ‘cleared space’) of the plasmonic hotspot formed by the plasmonic nanoantenna described herein, whilst at the same time retaining the biological activity of the nucleic acid polymerase. e.g., immobilisation of the nucleic acid polymerase to the surface of the nanoscopic nucleic acid scaffold is achieved by a nucleic acid linker. In one example, the nucleic acid linker which tethers the nucleic acid polymerase to the surface of the nanoscopic nucleic acid scaffold (e.g., the nanoscopic DNA scaffold described herein) is a double-stranded DNA linker comprising (i) a DNA polynucleotide which is covalently or non-covalently bound to the nucleic acid polymerase via an amino acid within the nucleic acid polymerase, and (ii) a DNA polynucleotide forming part of, or extending from, the surface of the nanoscopic nucleic acid scaffold, wherein the polynucleotides at (i) and (ii) each comprise single-stranded DNA sequences which are complementary or substantially complementary to one another and are capable of hybridizing to one another. The polynucleotide bound to the nucleic acid polymerase may comprise a peptide tag, which is in turn bound to nucleic acid polymerase via an isopeptide bond formed between an amino acid within the nucleic acid polymerase and an amino acid within the peptide tag. In the present case, the inventors employed a SpyCatcher-SpyTag bioconjugation system as described in Hatlem et al (2019) Int. J. Mol. Sci, 20(9):2129 to attach the DNA polynucleotide of the double-stranded DNA linker to the nucleic acid polymerase. In accordance with an example in which a SpyCatcher-SpyTag system is used, the polymerase is modified to express a SpyCatcher protein on its surface. The cognate SpyTag is then conjugated to the polynucleotide by any means known in the art. Methods of producing oligonucleotide-peptide conjugates are known in the art, such as described in Klabenkova et al (2021) Molecules, 26(17):5420, and include (but are not limited to) conjugation through thioether or disulfide bond, native ligation, oxime linkage, thiazolidine linkage or hydrazone linkage, amine bond formation, click chemistry, Diels-Alder reaction, and thiol-maleimide conjugation. However, in one particular example, the DNA polynucleotide is modified to comprise a maleimide and the SpyTag is conjugated to the DNA polynucleotide through thiol-maleimide conjugation (e.g., as described in the Examples herein). Once the DNA polynucleotide comprising the SpyTag is produced, it can be contacted with the polymerase modified to express a SpyCatcher protein. Upon recognition, the SpyCatcher and SpyTag form a covalent isopeptide bond between the side chains of a lysine in SpyCatcher and an aspartate in SpyTag, such that the polymerase and the DNA polynucleotide of the double-stranded DNA linker are bound. Although the SpyCatcher-SpyTag system was used herein, the skilled person would appreciate that alternative enzyme-mediate conjugation systems (including variant Catcher-Tag systems) may be employed and may rely on alternative amino acid combinations known for forming isopeptide bonds to covalently bind the polymerase and the DNA polynucleotide of the double-stranded DNA linker. The skilled person would also be aware of alternative chemistries for protein ligation and bioconjugation which may be employed, and these are contemplated herein.

In some examples, the amino acid within the polymerase to which the polynucleotide linker is bound is located with the N-terminal domain of the polymerase.

Each of the DNA polynucleotides comprised within the double-stranded DNA linker which binds or immobilises the nucleic acid polymerase on the nanoscopic nucleic acid scaffold, comprises a region of sufficient length and complementarity to hybridise to one another. Each DNA polynucleotide comprised within the double-stranded DNA linker may be between about 20 nucleotides to about 50 nucleotides in length (e.g., about 20 to about 30 nucleotides in length or about 20 to about 40 nucleotides in length, or about 30 to about 40 nucleotides in length). In one example, the DNA polynucleotides in the double-stranded DNA linker are between about 20 to about 30 nucleotides in length. Preferably, the region of Watson-Crick pairing between the respective DNA polynucleotides comprised within the double-stranded DNA linker binding or immobilising the polymerase on the nanoscopic nucleic acid scaffold is from about 8-30 nucleotides in length, or between about 10-25 nucleotides in length, or about 12-20 nucleotides in length. In this regard, the skilled person can adjust the length of the region of Watson-Crick pairing within the double-stranded DNA linker to accomplish a desired binding specificity and/or binding strength and/or a desired hybridization efficiency.

The DNA polynucleotides comprised within the double-stranded DNA linker which binds or immobilises the nucleic acid polymerase on the nanoscopic nucleic acid scaffold, may comprise one or more modifications. Suitable modifications are known in the art. They include for example Azo-modifications known in the art which can be used to realize photoresponsive and reversible binding and unbinding of the respective DNA sequences. Further modifications include modifications that modulate the strength of the interaction between the respective DNA sequences, such as backbone modifications that alter the surface charge (e.g. peptide nucleic acids (PNA)) or structural flexibility to induce hybridization efficiency (e.g. locked nucleic acids (LNA)). Even further modifications include the use of photocleavable structures known in the art.

In each of the foregoing examples describing the plasmonic nanoantenna of the disclosure, the nucleic acid polymerase may be a DNA polymerase.

The term “DNA polymerase” or “DNA polymerase molecule” as used herein refers to an enzyme that synthesizes a new strand of DNA in a 5′ to 3′ direction from a primer hybridized to a template strand of DNA. DNA polymerases are well-known in the art and commercially available. The DNA polymerase “reads” the template in the 3′→5′ direction and adds individual nucleotides (bases) to the new or nascent strand in the 5′→3′ direction. The polymerase requires a 3′OH group from a primer to begin extension of a new DNA strand. Individual nucleotides (dNTPs, or dATP, dCTP, dTTP, dGTP, or A, C, T, G) are added to the extending DNA molecule iteratively as the new DNA strand is synthesized by DNA polymerase. The particular base (A, C, T, or G) depends on the sequence of the template DNA, so that the new base hybridizes to the nucleotide on the template strand through a Watson-Crick interaction. The DNA polymerase cycles between “open” and “closed” conformations. The DNA polymerase is in the open position with the primer-template DNA complex. Once an incoming nucleotide enters the active site, the polymerase cycles to the closed position.

A variety of DNA polymerases can be used, many of which are commercially available. At least five families of DNA-dependent DNA polymerases are known, although most fall into families A, B and C. Most family A polymerases are single chain proteins that can contain multiple enzymatic functions including polymerase, 3′ to 5′ exonuclease activity and 5′ to 3′ exonuclease activity. Family B polymerases typically have a single catalytic domain with polymerase and 3′ to 5′ exonuclease activity, as well as accessory factors. Family C polymerases are typically multi-subunit proteins with polymerizing and 3′ to 5′ exonuclease activity. In E. coli, three types of DNA polymerases have been found, DNA polymerases I, II, and III (analogous to family A, B, and C, respectively). In eukaryotic cells, three different family B polymerases, DNA polymerases α, δ, and ∈ are implicated in nuclear replication, and a family A polymerase, polymerase γ, is used for mitochondrial DNA replication. Other types of DNA polymerases include phage polymerases. Any of these polymerases, combinations of all or portions of these polymerases, as well as chimeras or hybrids between two or more of such polymerases or their equivalents can be used to form a portion or all of the DNA polymerase molecule of the plasmonic nanoantenna described herein.

Examples of DNA polymerases that can be used include without limitation: phi-29, Taq, T7, E. coli Klenow (from DNA pol I), E. coli DNA pol III, and Bacillus stearothermophilus (Bst) DNA pol. The DNA polymerase can also be genetically engineered, e.g., a hybrid (e.g., Phusion DNA polymerase in which a domain with strong dsDNA binding affinity is fused to a DNA polymerase to enhance processivity). Many useful DNA polymerases are commercially available (e.g., T7 DNA pol, Sequenase version 2.0™). Highly processive polymerases include phi29 and T7 DNA polymerases, and Moloney murine leukemia virus (M-MLV) reverse transcriptase. One of skill in the art will appreciate that DNA polymerases are structurally similar, and that recombinant, hybrid polymerases can be engineered using homologous domains from different polymerases.

In some examples, to avoid the potential for differences in the incorporation efficiency of fluorescently labelled dNTPs, a DNA polymerase and labels that maximize incorporation efficiency and minimize incorporation variability can be used. Several DNA polymerases can efficiently incorporate fluorescently labelled dNTPs along DNA templates to produce very uniform fragments independent of the sequence context, and these polymerases are used in most automated Sanger/dideoxy-based methods using capillary-array DNA sequencers. A number of natural and engineered DNA polymerases are capable of incorporating fluorescently-labelled dNTPs (Reeve & Fuller Nature 376, 796-97 (1995); Rosenblum et al., Nucleic Acids Res 25, 4500-04 (1997); Ramanathan et al. Anal Biochem 337, 1-11 (2005); Aksyonov et al., Anal Biochem 348, 127-138 (2006); Tabor & Richardson, J Biol Chem 265, 8322-28 (1990); Zhu et al., Nucleic Acids Res 22, 3418-22 (1994); Zhu & Waggoner Cytometry 28, 206-211 (1997); Randolph & Waggoner Nucleic Acids Res 25, 2923-29 (1997); Mitra et al., Anal Biochem 320, 55-65 (2003); Anderson et al. Biotechniques 38, 257-264 (2005)).

As described hereinabove, the DNA polymerase may be modified to express an amino acid sequence (e.g., a CatcherTag or other binding motif) which is capable of binding to a cognate peptide tag (e.g., a SpyTag of other tag recognized by a binding motif) conjugated to a DNA polynucleotide within the nucleic acid linker (e.g., the double-stranded DNA linker) described herein for anchoring the polymerase the surface of the nanoscopic nucleic acid scaffold.

In some examples, the plasmonic nanoantenna described herein may be immobilised on a substrate via its nucleic acid scaffold. In particular examples the substrate is a solid substrate. For example, the plasmonic nanoantenna described herein may be immobilised on glass or silica. For example, the plasmonic nanoantenna described herein may be immobilised on a glass or silica slide or glass or silica chip. However, other solid substrates will be known to the skilled person and are contemplated herein. In some examples, the substrate may be coated with an antifouling material.

### Arrays of Plasmonic Nanoantenna

The present disclosure also provides an array comprising a plurality of the plasmonic nanoantenna described herein. According to this example, a plurality of the plasmonic nanoantenna of the disclosure may be immobilised on a substrate. For example, each plasmonic nanoantenna of the array may be immobilised on the solid substrate via its nucleic acid scaffold.

Each plasmonic nanoantenna may be immobilised on a solid substrate via the nanoscopic DNA scaffold using means known in the art. For example, the nanoscopic DNA scaffold may be immobilised to the solid substrate via a DNA linker or via a DNA origami structure. However, the skilled person will appreciate that there are many ways of immobilizing biological analytes, such the plasmonic nanoantenna of the disclosure, onto a substrate, whether covalently or non-covalently, via a linker moiety, or tethering them to an immobilized moiety. These methods are well known in the field of solid phase synthesis and micro-arrays (Beier et al., Nucleic Acids Res. 27:1970-1-977 (1999)). Non-limiting exemplary binding moieties for attaching either nucleic acids and proteins to a solid support include streptavidin or avidin/biotin linkages, carbamate linkages, ester linkages, amide, thiolester, (N)-functionalized thiourea, functionalized maleimide, amino, disulfide, amide, hydrazone linkages, among others. In addition, a silyl moiety can be attached to a nucleic acid directly to a substrate such as glass using methods known in the art. Additional methods for immobilization are provided in U.S. Ser. Nos. 11/645,125 and 11/645,135, both of which were filed Dec. 21, 2006; and U.S. Patent Publication No. 20080199932, all of which are incorporated herein by reference in their entireties for all purposes. In one particular non-limiting example, an avidin/biotin linkage is used to immobilize each plasmonic nanoantenna to the surface of the substrate (e.g., a glass or silica slip), whereby a biotin molecule is conjugated to each plasmonic nanoantenna (e.g., optionally via a linker) and the nanoantenna is immobilized on the substrate via surface-immobilised avidin (e.g., streptavidin).

The array may comprise a plurality of the plasmonic nanoantenna spaced apart on the substrate (e.g., at regular intervals) to a desired density and in a desired pattern.

The substrate may be formed from any suitable material to which the plasmonic nanoantenna can be covalently or non-covalently attached including, for example, glass, nylon, carbohydrates such as dextrans, plastic such as polystyrene, or polypropylene, polyacrylamide, latex, silicon, paper, metals such as gold, chromium, titanium, or tin, titanium oxide, tin oxide, or cellulose. According to a particular example, the substrate is glass, such as a glass slide or chip. Any suitable glass may be used but a preferred substrate has at least a silicon dioxide surface layer. In another specific examples, the substrate is a flexible plastic. For example, the flexible plastic may comprise nitrocellulose acetate. The flexible plastic substrate may also comprise a flexible plastic strip. The use of flexible plastic substrates may permit greater portability and also renders the array more adaptable for interfacing with other devices, for example wrapping around optical fibers and optical cables for increased sensitivity.

The substrate may further be provided in any desired form, including in the form of a chip, a bead, a well, a flowcell, a nanowell, a capillary tube, a slide, a wafer, a filter, a fiber, a porous media, a porous nanotube, or a column.

In some examples, the assay may be configured for simultaneously assaying (e.g., sequencing) two or more samples. In this regard the solid substrate onto which the plasmonic nanoantenna are immobilised may be partitioned, with each region comprising an array of distinct plasmonic nanoantenna. For example, the substrate may be a microwell plate or microchannel plate. The microwell plate or microchannel plate may also be configured to be read by a standard plate reader, e.g., a fluorometer.

In some examples, the surfaces of the substrate may be modified to allow or enhance covalent or non-covalent attachment of plasmonic nanoantenna described herein. The substrate and process for attachment of the plasmonic nanoantenna thereto are preferably stable for repeated binding, washing, imaging and eluting steps (if desired). In some examples, the surface of the substrate may be modified to have a positive or negative charge. In some examples, the surface of the substrate may be functionalized by modification with specific functional groups, such as maleic or succinic moieties, or derivatized by modification with a chemically reactive group, such as amino, thiol, or acrylate groups, such as by silanization. Suitable silane reagents include aminopropyltrimethoxysilane, aminopropyltriethoxysilane and 4-aminobutyltriethoxysilane. The surfaces may be functionalized with N-Hydroxysuccinimide (NHS) functional groups. Glass surfaces can also be derivatized with other reactive groups, such as acrylate or epoxy, using, e.g., epoxysilane, acrylatesilane or acrylamidesilane.

In some examples, the solid substrate may be modified to reduce non-specific attachment of plasmonic nanoantenna to the surface of the solid substrate. In some examples, the solid support may be modified to reduce non-specific binding of biological entities and/or chemical entities to the surface of the substrate. For example, the surface of the substrate may be anti-fouled to prevent non-specific adsorption thereto. Suitable anti-fouling reagents are known in the art and contemplated herein. However, in one particular example, the surface of the substrate is treated with APTES. In some examples, the substrate may be passivated and/or the surface of the substrate may be passivated. For example, the passivation layer may include diamond-like carbon, hexa-methyldisilizane, Teflon, fluorocarbon, a polymer such as polyethylene glycol (PEG) and/or Parylene. In some examples, a substrate may be passivated by the attachment of Polyethylene glycol (PEG) molecules across the substrate. In some examples, passivation components may not be covalently bound to a surface of the substrate. For example, a substrate may be passivated by coating with Bovine Serum Albumin (BSA) or caseins (e.g., from dry milk powder).

In some examples, the solid substrate may be modified across the entire surface to which the plasmonic nanoantenna are to be attached. In other examples, the surface of solid substrate may contain regions which are modified to allow attachment of the plasmonic nanoantenna and regions which are not modified. Alternatively, or in addition, the surface of solid substrate may contain regions which are modified to decrease attachment of plasmonic nanoantenna and regions which are not modified. Alternatively, or in addition, the surface of solid substrate may contain regions which are modified to increase attachment of plasmonic nanoantenna and regions which are modified to decrease attachment of the plasmonic nanoantenna. In some cases, attachment sites for the plasmonic nanoantenna may be created in an array, for example an ordered array.

An ordered array of attachment sites may be created by, for example, photolithography, Dip-Pen nanolithography, nanoimprint lithography, nanosphere lithography, cluster lithography, nanopillar arrays, nanowire lithography, scanning probe lithography, thermochemical lithography, thermal scanning probe lithography, local oxidation nanolithography, molecular self-assembly, stencil lithography, or electron-beam lithography. Attachment sites in an ordered array may be located in any density and/or configuration. For example, attachment sites in an ordered array may be located such that each attachment site is less than 20 nm, or about 20 nm, or about 50 nm, or about 75 nm, or about 100 nm, or about 125 nm, or about 150 nm, or about 175 nm, or about 200 nm, or about 225 nm, or about 250 nm, or about 275 nm, or about 300 nm, or about 325 nm, or about 350 nm, or about 375 nm, or about 400 nm, or about 425 nm, or about 450 nm, or about 475 nm, or about 500 nm, or about 525 nm, or about 550 nm, or about 575 nm, or about 600 nm, or about 625 nm, or about 650 nm, or about 675 nm, or about 700 nm, or about 725 nm, or about 750 nm, or about 775 nm, or about 800 nm, or about 825 nm, or about 850 nm, or about 875 nm, or about 900 nm, or about 925 nm, or about 950 nm, or about 975 nm, or about 1000 nm, or about 1025 nm, or about 1050 nm, or about 1075 nm, or about 1100 nm, or about 1125 nm, or about 1150 nm, or about 1175 nm, or about 1200 nm, or about 1225 nm, or about 1250 nm, or about 1275 nm, or about 1300 nm, or about 1325 nm, or about 1350 nm, or about 1375 nm, or about 1400 nm, or about 1425 nm, or about 1450 nm, or about 1475 nm, or about 1500 nm, or about 1525 nm, or about 1550 nm, or about 1575 nm, or about 1600 nm, or about 1625 nm, or about 1650 nm, or about 1675 nm, or about 1700 nm, or about 1725 nm, or about 1750 nm, or about 1775 nm, or about 1800 nm, or about 1825 nm, or about 1850 nm, or about 1875 nm, or about 1900 nm, or about 1925 nm, or about 1950 nm, or about 1975 nm, or about 2000 nm, or more than 2000 nm from any other attachment site.

In some examples, the spacing of attachment sites on the surface of the solid substrate may be selected depending on the size of the plasmonic nanoantenna to be attached.

In some examples, less than 30% of the plasmonic nanoantenna in the array are clustered in aggregates of two plasmonic nanoantenna or more. For example, less than about 25% of the plasmonic nanoantenna in the array are clustered in aggregates of two plasmonic nanoantenna or more. For example, less than about 20% of the plasmonic nanoantenna in the array are clustered in aggregates of two plasmonic nanoantenna or more. For example, less than about 15% of the plasmonic nanoantenna in the array are clustered in aggregates of two plasmonic nanoantenna or more. For example, less than about 10% of the plasmonic nanoantenna in the array are clustered in aggregates of two plasmonic nanoantenna or more.

In some examples, the substrate may be optically opaque. In some examples, the substrate may be optically clear at one or more wavelengths. In some examples, the substrate may be partially optically clear or may be optically clear in some regions. For example, a solid substrate may be optically opaque in regions that are not functionalized, and optically clear in regions that are functionalized.

### Methods of DNA Sequencing

The inventors have shown for the first time that a DNA polymerase molecule can be positioned within an area of electrical field enhancement when sequencing a single DNA molecule, such that it is possible to detect incorporation of specific, fluorescently labelled dNTPs into a growing strand of DNA by the DNA polymerase enzyme during DNA synthesis.

Accordingly, in one example, the disclosure provides method of sequencing a nucleic acid analyte, comprising:
(I) contacting a nucleic acid polymerase with the nucleic acid analyte and labelled nucleotides for a time and under conditions such that the labelled nucleotides are sequentially incorporated by the polymerase into a polynucleotide having a sequence which is complementary to a polynucleotide sequence of the nucleic acid analyte, wherein the polymerase is positioned within an area of electrical field enhancement; and each labelled nucleotide comprises:
   (i) an adenine nucleotide (A), a guanosine nucleotide (G), a thymine nucleotide (T), or a cytosine nucleotide (C),
   (ii) a fluorophore, and
   (iii) a polyphosphate linker binding the nucleotide to the fluorophore;
   wherein the A, G, T and C are each independently linked to a fluorophore via the polyphosphate,
   wherein each of labelled nucleotides A, G, T and C has a distinct fluorescence emission signature when the fluorophore to which the respective nucleotide is linked is excited, and
   wherein the distinct fluorescence emission signatures of the labelled nucleotides are enhanced upon their incorporation by the nucleic acid polymerase into the sequence which is complementary to a polynucleotide sequence of the nucleic acid analyte; and
(II) detecting the order of enhanced distinct fluorescence emission signatures as the labelled nucleotides are sequentially incorporated by the nucleic acid polymerase into the polynucleotide having a sequence which is complementary to a polynucleotide sequence of the nucleic acid analyte, and thereby determining the sequence of the nucleic acid analyte by determining the order of nucleotides incorporated into the polynucleotide having a sequence which is complementary to a polynucleotide sequence of the nucleic acid analyte.

In order for fluorophores attached to the nucleotides to emit a fluorescence signal when co-localised with the polymerase, a source of electromagnetic energy must be provided to the sequencing reaction. Accordingly, the method comprises providing a source of electromagnetic energy to the labelled nucleotides when co-localised with the DNA polymerase. In one example, the electromagnetic energy is provided by one or more lasers. The one or more lasers will be configured to provide electromagnetic energy (e.g., light) to the labelled nucleotides at a wavelength corresponding to the excitation wavelength of the fluorophores attached to the nucleotides. As used herein, the phrase “area of electrical field enhancement” refers to an area or region in which the electrical field is enhanced, relative to the electrical field outside the defined area. In some cases, and in the case of DNA sequencing, enhancement of the electrical field is due to plasmon resonance induced by the incoming oscillating electric field (e.g. light).

In some examples, the area of electrical field enhancement is produced by a plasmonic hotspot, wherein the plasmonic hotspot is produced by a plasmonic nanoantenna as described herein. Accordingly, in some examples, the method comprises using the plasmonic nanoantenna described herein to sequence a nucleic acid analyte according to the method described herein. In accordance with this example, the step (I) of the method comprises contacting the nucleic acid analyte and labelled nucleotides with the plasmonic nanoantenna described herein for a time and under conditions sufficient for the labelled nucleotides to be sequentially incorporated by the nucleic acid polymerase, wherein the polymerase is bound to the nanoscopic nucleic acid (e.g., DNA) scaffold and is positioned within the region of the plasmonic hotspot which is unoccupied by the nanoscopic nucleic acid scaffold.

In one example, the method comprises contacting the nucleic acid polymerase with the nucleic acid analyte in the presence of each of the labelled nucleotides A, G, T and C. In this regard, the method preferably comprises detecting incorporation of each type of naturally occurring nucleotide and allowing incorporation of each nucleotide to be discriminated from incorporation of other types of nucleotides, thereby providing a nucleotide sequence of the nucleic acid analyte. In other examples, the nucleotide of the labelled nucleotide may be further selected from a synthetic nucleotide. In one example, the synthetic nucleotide is selected from 5-methylcytidine, N-methylcytidine, and N6-methyladenosine. Accordingly, the method may allow the incorporation of synthetic nucleotides as described herein to be discriminated from incorporation of other types of nucleotides when determining the nucleotide sequence of the nucleic acid analyte.

The method further comprises contacting the nucleic acid analyte, which is a DNA molecule, with an oligonucleotide primer which is capable of hybridising specifically to a region of the DNA to initiate synthesis by the nucleic acid polymerase e.g., DNA polymerase.

The terms “primer” or “oligonucleotide primer” or similar as used in the context of methods of DNA sequencing shall be understood to mean an oligonucleotide or nucleic acid fragments which is capable of hybridising specifically to a template DNA sequence of the DNA molecule to initiate synthesis using a DNA polymerase. A primer can be of any length depending on the particular technique it will be used for. However, primers for priming synthesis of DNA in a DNA polymerase reaction (e.g., PCR) are generally between 10 and 40 nucleotides in length. One of skill could readily design a primer having a sequence of suitable complementarity to hybridise specifically to a template DNA sequence of interest using methods and software known in the art. In some examples, the oligonucleotide primer is designed to be substantially complementary to a universal adapter template sequence which has been attached to (e.g., through ligation) the 3′ end of the template DNA sequence. In other examples, the oligonucleotide primer is designed to be substantially complementary to a hairpin adapter sequence which have been attached to (e.g., through ligation) the 3′ end and 5′ end of the template DNA sequence. The use of universal primers means only one primer sequence is required to initiate DNA synthesis of any number of DNA molecules. In accordance with examples in which a library of DNA molecules or fragments there are prepared for sequencing, each of those DNA molecules or fragments thereof may have a universal adapter template sequence attached to (e.g., through ligation) the 3′ end to provide a template for a universal primer to specifically hybridise to.

The term “selectively” or “specifically” as used in the context of hybridisation refers to the binding, duplexing, or hybridizing of an oligonucleotide primer to a particular (and usually predetermined) location within a template DNA sequence with a higher affinity, e.g., under more stringent conditions, than to other (non-specific) locations within a template DNA sequence. One of skill in the art will appreciate that specific hybridization between nucleotides usually relies on Watson-Crick pair bonding between complementary nucleotide sequences.

The nucleic acid polymerase is preferable a DNA polymerase. As used herein, the term “DNA polymerase” refers to an enzyme that synthesizes a new strand of DNA in a 5′ to 3′ direction from a primer hybridized to a template strand of DNA. The DNA polymerase “reads” the template in the 3′→5′ direction and adds individual nucleotides (bases) to the new or nascent strand in the 5′→3′ direction. The polymerase requires a 3′OH group from a primer to begin extension of a new DNA strand. Individual nucleotides (dNTPs, or dATP, dCTP, dTTP, dGTP, or A, C, T, G) are added to the extending DNA molecule iteratively as the new DNA strand is synthesized by DNA polymerase. The particular base (A, C, T, or G) depends on the sequence of the template DNA, so that the new base hybridizes to the nucleotide on the template strand through a Watson-Crick interaction. The DNA polymerase cycles between “open” and “closed” conformations. The DNA polymerase is in the open position with the primer-template DNA complex. Once an incoming nucleotide enters the active site, the polymerase cycles to the closed position. DNA polymerases are well-known in the art and a wide range is commercially available. Exemplary DNA polymerase enzymes which may be used in the method of the disclosure are described hereinabove in the context of the plasmonic nanoantenna and shall be taken to apply mutatis mutandis to each and every example describing the method of sequencing unless stated otherwise.

As used herein, the term “labelled nucleotides” refers to any nucleotide (including naturally occurring and non-naturally occurring or “synthetic nucleotides”) attached directly or indirectly to a fluorophore. The terms “fluorophore”, “fluorescent label” or similar refer to a signaling moiety that conveys information through the fluorescent absorption and/or emission properties of one or more molecules. Such fluorescent properties include fluorescence intensity, fluorescence lifetime, emission spectrum characteristics, energy transfer and the like.

The fluorophore is bound to the nucleotide via a polyphosphate linker. In one example, the polyphosphate is a tri-, tetra-, penta- or hexa-phosphate. For example, the polyphosphate is a triphosphate. For example, the polyphosphate is a tetra-phosphate. For example, the polyphosphate is a penta-phosphate. For example, the polyphosphate is a hexa-phosphate.

In some examples, the fluorophore is bound to the phosphate furthest from the nucleotide.

Exemplary fluorophores which may be conjugated or attached to nucleotides used in the sequencing method include, but not limited to fluorescein/Oregon Green, fluorescein isothiocyanate (FITC), 6-Carboxyfluorescein, tetramethylrhodamine, Texas Red, dansyl, Alexa Fluor 488, BODIPY FL, lucifer yellow, and Alexa Fluor 405/Cascade Blue fluorophores. Commercially available fluorescent nucleotide analogues which may be readily incorporated into the a DNA molecule during a DNA polymerase synthesis include, for example, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, ALEXA FLUOR™ 546-14-UTP (Molecular Probes, Inc. Eugene, Oreg.). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000).

Other fluorophores available for post-synthetic attachment to dNTPs include, inter alia, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like) (available from Thermo Fisher Scientific, Rockford, Ill.), Texas Red (available from Molecular Probes, Inc., Eugene, Oreg.), and Cy2, Cy3.5, Cy5.5, and Cy7 (available from Amersham Biosciences, Piscataway, N.J. USA, and others).

The fluorophore(s) linked to the nucleotides may have a low quantum efficiency and/or yield. In this regard, compared with fluorophores which are strong emitters, poor emitters with low quantum yields may benefit from the strong fluorescent enhancement within the plasmon resonance hotspot. The use of fluorophores having low quantum yield may aid in reducing background noise from fluorescently labelled nucleotides outside of the area of electrical field enhancement which are not be incorporated into the new DNA strand.

The term “fluorescence emission signature”, as used herein, refers to cumulative fluorescence properties of the labelled nucleotides when excited during a process of sequencing a nucleic acid analyte as described herein including, but not limited to, peak emission wavelength, fluorescence emission intensity, and fluorescence emission duration. Accordingly, it follows that a “distinct fluorescence emission signature” is one which can be distinguished from another fluorescence emission signature when labelled nucleotides are excited. For example, a “distinct fluorescence emission signature” as described herein may be a fluorescence emission signature for a labelled nucleotide which is distinguishable from another labelled nucleotide used in the sequencing method described herein based on differences in one or more of differences in peak emission wavelength, differences in fluorescence emission intensity, differences in fluorescence emission duration, differences in duration between successive fluorescence emissions, or any combinations thereof.

In some examples, the distinct fluorescence emission signatures are based on peak emission wavelength and two or more (e.g., 2, or 3 or 4) species of the labelled nucleotides are distinguishable based on differences in peak emission wavelength. In one example, each different species of nucleotide is linked to a fluorophore having a distinct peak emission wavelength. In accordance with any example in which labelled nucleotides are distinguishable based on differences in peak emission wavelength, the peak emission wavelengths of the labelled nucleotides are separated by 10 nm or more relative to each other. For example, the peak emission wavelengths may be separated by about 25 nm or more relative to each other. For example, the peak emission wavelengths may be separated by about 50 nm or more relative to each other. For example, the peak emission wavelengths may be separated by about 75 nm or more relative to each other. For example, the peak emission wavelengths may be separated by about 100 nm or more relative to each other. In certain examples, each different species of nucleotide is linked to a fluorophore having a distinct peak emission wavelength and the peak emission wavelengths of the respective fluorophores is separated by about 100 nm or more relative to each other.

Each labelled nucleotides may include a fluorophore having a peak emission wavelength independently selected from an emission wavelength in the visible spectrum, an emission wavelength in the ultraviolet (UV) spectrum, an emission wavelength in the infrared (IR) spectrum, and an emission wavelength in the near-IR spectrum. In one example, one or more or each fluorophore has a peak emission wavelength independently selected from an emission wavelength between about 350 nm and 850 nm.

Alternatively, or in addition, different species of nucleotides are distinguished from one another based on differences in fluorescence emission intensity. For example, differences in fluorescence intensity may be achieved by differences in the amount of a fluorophore (e.g., the same fluorophore or a different fluorophore) linked to different species of nucleotides. In accordance with this example, two or more species of labelled nucleotides used in the method may labelled with the same fluorophore and are distinguishable based on differences in fluorescence emission intensity. In one example, two or more species of labelled nucleotides used in the method are distinguishable based on differences in fluorescence emission intensity.

In another example, different species of nucleotides are distinguished from one another based on differences in fluorescence emission duration. In this regard, different nucleotides are known to have different dwell times when being incorporated into the growing DNA strand by DNA polymerase during synthesis. These differences in dwell time impact the during of fluorescence emission from the fluorophore linked to the nucleotide. Accordingly, in some examples, the distinct fluorescence emission signature by which a species of nucleotide is identified during the method of the disclosure may including a fluorescence emission duration which are unique to the species of nucleotide, or distinct to species of nucleotide in combination with a specific fluorophore. In accordance with this example, two or more species of labelled nucleotides used in the method may be distinguished based on differences in fluorescence emission duration.

As described herein, the distinct fluorescence emission signature of the respective labelled nucleotide being incorporated by the nucleic acid polymerase into the growing DNA sequence is enhanced relative to the fluorescence emission signature of corresponding labelled nucleotides outside of the area of electrical field enhancement. In one example, the distinct fluorescence emission signature of the labelled nucleotide being incorporated by the polymerase is enhanced by two times or more (e.g., by about three times or more, or by about four times or more, or by about five times or more, or by about six times or more, or by about seven times or more, or by about eight times or more or by about nine times or more) as compared to the fluorescence emission signatures of corresponding labelled nucleotide outside of the area of electrical field enhancement. In some examples, the distinct fluorescence emission signature of the labelled nucleotide of the labelled nucleotide being incorporated by the polymerase is enhanced by an order of magnitude or more (e.g., about 10× or more, or about 20× or more, or about 30× or more, or about 40× or more, or about 50× or more, or about 60× or more, or about 70× or more, or about 80× or more, or about 90× or more, or about 100× or more) as compared to the fluorescence emission signature of corresponding labelled nucleotide outside of the area of electrical field enhancement. In further examples, the distinct fluorescence emission signature of a labelled nucleotide being incorporated by the polymerase is enhanced by at least about 100× or more (e.g., at least about 150×, or at least about 200×, or at least about 250×, or at least about 300×, or at least about 350×, or at least about 400×, or at least about 450×, or at least about 500×, or at least about 550×, or at least about 600×, or at least about 650×, or at least about 700×, or at least about 750×, or at least about 800×, or at least about 850×, or at least about 900×, or at least about 950×, or at least about 1000×) as compared to the fluorescence emission signature of a corresponding labelled nucleotide outside of the area of electrical field enhancement.

In some examples, the method further comprises contacting the nucleic acid polymerase with the nucleic acid analyte and labelled nucleotides in the presence of one or more quenching agents. As used herein, “a quenching agent” or “quencher” refers to any fluorescence-modifying moiety that can attenuate or reduce the light emitted by a fluorophore. This attenuation or reduction in emission of light is referred to as “quenching”. Accordingly, in some examples, one or more quenching agents may be provided to assist in reducing background noise from fluorophores linked to labelled nucleotides which are outside of the area of electrical field enhancement. In some examples, the quenching agents are provided in a free, unconjugated form. Quenchers which may be useful in the methods of the disclosure include, without limitation, Black Hole Quencher Dyes (Biosearch Technologies such as BHQ-0, BHQ-1, BHQ-2, BHQ-3, BHQ-10; QSY Dye fluorescent quenchers (from Molecular Probes/Invitrogen) such as QSY7, QSY9, QSY21, QSY35, and other quenchers such as Dabeyl and Dabsyl; Cy5Q and Cy7Q and Dark Cyanine dyes (GE Healthcare), which can be used, for example, in conjunction with donor fluors such as Cy3B, Cy3, or Cy5; DY-Quenchers (Dyomics), such as DYQ-660 and DYQ-661; and ATTO fluorescent quenchers (ATTO-TEC GmbH), such as ATTO 540Q, 580Q, 612Q.

The term “nucleic acid analyte” refers to any nucleic acid molecule, the sequence of which is to be determined using the method of the disclosure. In its broadest form, a “nucleic acid” refers to a polymer having multiple nucleotide monomers. A nucleic acid analyte can be single- or double-stranded, and can be DNA (e.g., complementary DNA, genomic DNA or mitochondrial DNA), RNA, or hybrid polymers (e.g., DNA/RNA). The term “nucleic acid” does not refer to any particular length of polymer. Rather, a nucleic acid may be any length e.g., greater than about 2 bases, greater than about 10 bases, greater than about 100 bases, greater than about 500 bases, greater than 1000 bases, greater than 10,000 bases, greater than 100,000 bases, greater than about 1,000,000 or more bases composed of nucleotides.

The nucleic acid analyte to be sequenced can be obtained from any source of interest, and can comprise DNA, mRNA, and mimetics, analogs, and derivatives thereof. They can be isolated from cells, cell cultures, tissue samples, bodily fluids, viral samples, genomic nucleic acid samples, cDNA preparations, environmental samples, forensic samples, or synthetic sources. Nucleic acids can be cloned, amplified, transcribed, ligated, fragmented, or otherwise manipulated according to standard methods to provide the nucleic acid to be sequenced as these manipulations do not render the nucleic acid unsuitable for subsequent sequencing as described herein. It will be understood that such nucleic acids may comprise modified, non-canonical, and/or non-natural nucleotides or nucleotide analogs, many of which are described in U.S. patent application Ser. No. 12/945,767, filed Nov. 12, 2010, which is incorporated herein by reference in its entirety for all purposes. Accordingly, the method of the disclosure may be used in a range of sequencing applications including, but not limited to, genome sequencing, transcriptome sequencing, RNA-sequencing, single-molecule sequencing, single-cell sequencing, methylation sequencing and combinations thereof.

In each of the foregoing examples, the nucleic acid analyte is sequenced by determining the order of nucleotides in a DNA molecule. In one example, the DNA is genomic DNA. In another example, the DNA is complementary DNA (cDNA) which has been derived from an RNA. In accordance with examples in which the nucleic acid analyte is cDNA, the method may comprise determining the sequence of an RNA based on the sequence for a corresponding cDNA.

In each of the foregoing examples, the nucleic acid analyte may be subjected to one or more processing steps prior to being localised with the plasmonic nanoantenna. These processing steps may include, but are not limited to, purification steps, reverse transcription (e.g., in the case of RNA), fragmentation to achieve desired size of template DNA, enrichment steps (e.g., for sequences or architecture of interest), depletion steps (e.g., to remove unwanted sequence), amplification steps (e.g., to increase the amount of template DNA), ligation steps (e.g., to attach adapters and other sequences of interest) and combinations thereof. The choice of processing steps and the techniques used may vary according to the application. In some examples, a combination of these processing steps may be employed to prepare one or more libraries (e.g., libraries of DNA templates) for sequencing using the method of the disclosure. The literature is replete with protocols and methods of preparing nucleic acid samples for sequencing reactions and a skilled person could readily determine the steps and methods required.

In some examples, the nucleic acid analyte is a double-stranded DNA molecule which may be denatured to produce a single stranded DNA molecule (i.e., a “target DNA sequence”, “template DNA”, or “template DNA sequence”) which can be used by the DNA polymerase enzyme to synthesize a new strand. The term “target DNA” or “template DNA” refers to a stretch of DNA which can be sequenced according to the present methods. The term encompasses both complementary strands of the target DNA molecule. One of skill will understand that the sequence of one strand of target DNA reveals the sequence of the other strand due to familiar Watson-Crick base pairing. The target DNA can be attached to one or more oligonucleotide primers or adaptors, e.g., to facilitate synthesis. However, in other examples, double-stranded DNA molecules are not denatured, but are instead retained in a double-stranded form.

The DNA molecules to be sequenced (including cDNA, single-stranded DNA and double-stranded DNA) may be fragmented or sheared into various sizes (0.5 kb to 10 kb) using hydrodynamic mechanical shearing with relatively narrow size distributions (Thorstenson et al., Genome Res 8, 848-855 (1998); Roe, Methods Mol Biol 255, 171-187 (2004)). Methods of fragmenting and/or shearing DNA are known in the art, including, e.g., sonication, nebulization, and enzymatic methods. Specialized devices for size-restricted DNA shearing are commercially available (e.g., Bioruptor® from Diagenode). These methods are further explicated in Sambrook (Molecular Cloning: A Laboratory Manual. New York: Cold Spring Harbor Laboratory Press; 1989) and Ausubel (Current Protocols in Molecular Biology. New York: John Wily; 2001), which are incorporated herein by reference in their entireties for all purposes.

The DNA molecule to be sequenced may be any length. For example, the DNA molecule to be sequenced may be 10 or more nucleotides in length, e.g. about 20 nucleotides in length, or about 50 nucleotides in length, or about 100 nucleotides in length, or about 200 nucleotides in length, or about 300 nucleotides in length, or about 400 nucleotides in length, or about 500 nucleotides in length, or about 1000 nucleotides in length, or more (e.g., 2000, or 5000, or 10,000 or more nucleotides). In some examples, the DNA molecule to be sequenced is between about 10-10000 nucleotides in length. In some examples, the DNA molecule to be sequenced is between about 100-5000 nucleotides in length. In some examples, the DNA molecule to be sequenced is between about 200-2000 nucleotides in length. In some examples, the DNA molecule to be sequenced is between about 400-1000 nucleotides in length. As described herein, in some examples a DNA molecule which is larger in size may be fragmented or sheared into various smaller sizes to facilitate sequencing.

The DNA molecules to be sequenced comprise one or more adapter sequences which provide a binding site for one or more oligonucleotide primers to hybridise and initiate synthesis by DNA polymerase. Accordingly, the method may comprise the further step of ligating adapters onto one or more (e.g., a library of) DNA molecules to be sequenced using the method described herein. For example, the method may comprise ligating a universal adapter sequence onto the 3′ end of the DNA molecules to be sequenced. In some examples, the method may comprise ligating hairpin adapters onto both ends of a double stranded DNA molecule to provide a sequencing template that comprises both the complementary and non-complementary strands of the DNA molecule in a single-stranded circular construct that can be repeatedly sequenced to provide redundant sequencing information from both strands. For single-molecule sequencing reactions, e.g., SMRT® Sequencing from Pacific Biosciences, where sequence data is generated from a single template molecule, statistical analysis of the redundant information is used to generate a consensus sequence for the target region from the single sequencing template. Further details about redundant sequencing and circular sequencing templates are provided, e.g., in U.S. Pat. Nos. 7,476,503 and 8,153,375, both of which are incorporated herein by reference in their entireties for all purposes.

In other examples, the method may comprise ligating hairpin adapters onto only one end of a double stranded DNA molecule. Ligation of hairpin adapters onto only one end of the double stranded DNA molecule provides a sequencing template that comprises both the complementary and non-complementary strands of the DNA molecules in a single-stranded linear construct that can be sequenced to provide a sequence read from each strand. Such a template is particularly beneficial in sequencing technologies in which a single-stranded linear template is preferred, e.g., in sequencing technologies that use a nanopore-based sensor, which have been described at length in the art and are currently being developed by several companies, including Oxford Nanopore and Genia. However, the use of template having stem-loop adapters at both ends are also contemplated for sequencing in a nanopore-based method, e.g., where the single-stranded circle produced by separating the complementary strands is used as a template for rolling-circle replication, e.g., where the nascent strand or the released phosphate groups are directed to or through the nanopore.

In each of the foregoing examples, step (II) of the method of sequencing a nucleic acid analyte comprises detecting the order of enhanced distinct fluorescence emission signatures as the labelled nucleotides are sequentially incorporated by the nucleic acid (e.g., DNA) polymerase into the polynucleotide having a sequence which is complementary to a polynucleotide sequence of the nucleic acid analyte (i.e., the complementary strand). The enhanced distinct fluorescence emission signatures may be detected by any means known in the art for detecting fluorescence, including but not limited to, confocal microscopy, confocal laser scanning microscopy, Total Internal Reflection (TIR), Total Internal Reflection Fluorescence (TIRF), epifluorescence microscopy, near-field scanning microscopy, far-field confocal microscopy, wide-field epi-illumination, light scattering, dark field microscopy, photoconversion, wide field fluorescence, single and/or multi-photon excitation, spectral wavelength discrimination, evanescent wave illumination, scanning two-photon, scanning wide field two-photon, Nipkow spinning disc, and/or multi-foci multi-photon. In some examples, a combination of these methods is used for detecting fluorescence. In one example, confocal laser scanning microscopy is used to detect the enhanced distinct fluorescence emission signatures of nucleotides as they incorporated by DNA polymerase into the complementary strand. In one example, Total Internal Reflection (TIR) is used to detect the enhanced distinct fluorescence emission signatures of nucleotides as they incorporated by DNA polymerase into the complementary strand. In one example, Total Internal Reflection Fluorescence (TIRF) is used to detect the enhanced distinct fluorescence emission signatures of nucleotides as they incorporated by DNA polymerase into the complementary strand.

The distinct fluorescence emission signatures emitted from the different species of labelled nucleotides may be resolved using any suitable discrimination methods which are based on: fluorescence resonance energy transfer measurements; photoconversion; fluorescent lifetime measurements; polarization; fluorescent lifetime determination; correlation/anti-correlation analysis; Raman; intensity; ratiometric; time-resolved methods; anisotropy; near-field or far field microscopy; fluorescence recovery after photobleaching (1-RAP); spectral wavelength discrimination; measurement and separation of fluorescence lifetimes; fluorophore identification; background suppression, parallel multi-color imaging, or any combination thereof. See, for example, J. R. Lakowitz 2006, in: “Principles of Fluorescence Spectroscopy”, Third Edition. If the different nucleotides are labeled with different energy transfer or reporter moieties, then resolving the emitted signals can be used to distinguish between the different nucleotides which bind the polymerase and/or which are incorporated by the polymerase. In one particular example, each of the labelled nucleotides emits a fluorescence signal in a different wavelength, such that the distinct fluorescence emission signatures of the nucleotide species are discriminated based on emission wavelength e.g., peak emission wavelength.

In order to detect distinct fluorescence emission signatures emitted from each of the different species of labelled nucleotide, the method may further employ a multifluorescence imaging system which is capable of detecting the multiple distinct fluorescence emission signatures emitted by the different species of labelled nucleotides during the course of the sequencing reaction. Such a system can include special filter combinations for each excitation line and/or each emission band. In one example, the detection system includes tunable excitation and/or tunable emission fluorescence imaging.

Where the method of the disclosure is performed on an array e.g., an array of plasmonic nanoantenna as described herein, the detection system may comprise an optical train which directs signals emitted from an organized array onto different locations of an array-based detector to detect multiple optical signals from multiple locations. The optical trains typically include optical gratings and/or wedge prisms to simultaneously direct and separate signals having differing spectral characteristics from different addressable locations in an array to different locations on an array-based detector, e.g., a CCD.

Following the detection of the order of distinct fluorescence emission signatures at step (II), the sequence of the nucleic acid analyte can be determined by correlating the order of distinct fluorescence emission signatures with the order of nucleotides sequentially incorporated into the DNA that was synthesized.

### Kits

The present disclosure also provides kits and reaction mixtures for conducting single nucleic acid molecule sequencing as described herein. The kit may vary in its components and their configuration but will generally include the plasmonic nanoantenna as described herein attached to a substrate or an array of said plasmonic nanoantennas as described, and a reaction mixture. The reaction mixture may comprise one or more fluorescently labelled nucleotides (e.g., such as each of dATP, dCTP, dGTP, dTTP as described herein), one or more buffers (e.g., Tris), various salts (e.g., KCl, NaCl, (NH₄)₂SO₄, MnCl₂, Zn salts, MgCl₂), and often stabilizer, detergent, DMSO, and DTT. Reaction mixtures of the disclosure can also include additives to increase the specificity and efficiency of polymerase reactions. The reaction mixture may include an oligonucleotide primer to primer DNA synthesis by DNA polymerase. In the case of a universal primer which hybridises to a universal adapter sequence, this can (but need not) be provided within the reaction mixture. In the case of bespoke primer sequences, it may be preferred that the primer be provided separately e.g., by the user. It will be appreciated that kits of the disclosure may encompass any combination of the above-described components.

A kit of the disclosure may also include instructions for performing the method of sequencing a single DNA molecule as described herein.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### EXAMPLES

### Example 1. General Materials and Methods

### DNA Origami Design and Synthesis

The inventors designed the DNA staple sequences for folding the M13mp18 single stranded DNA template into the DNA origami nanospanno scaffold using caDNAno.

### FEM Simulations for Estimating Fluorescence Enhancement

All simulations were performed in MATLAB 2020b using a toolbox for the simulation of metallic nanoparticles using a boundary element method approach (MNPBEM)²². The optical properties of silver and gold were obtained from Johnson and Christy²³ and the background medium permittivity was set to 1.77 to match experimental aqueous buffer. To simulate measurements on a total internal reflection fluorescence (TIRF) microscope, a glass substrate layer with a permittivity of 2.25 was included 5 nm below the nanoparticles on the z-axis, with an incident plane wave propagating at a 650 angle relative to the normal to the glass layer. Emission properties of dyes were modelled using as oscillating dipoles at a frequency corresponding to the maximum emission wavelength of the dye.

### DNA Origami Synthesis

DNA origami synthesis entails the folding of a single long template DNA strand comprised of the single-stranded phage vector M13mp18, by hybridisation with approximately 250 short ‘staple’ DNA strands that crosslink DNA helices at well-defined locations to form the desired shape^{21,24}. Synthesis requires an annealing process to prevent the formation of undesired kinetically-trapped structures and to maximise the yield of well-formed structures that are consistent with design. Synthesis conditions were optimised to the below conditions: 1× origami buffer (10 mM Tris pH 8 and 1 mM EDTA) plus 20 mM Magnesium Chloride (MgCl2), 15× staple excess (50× extension staple excess), 22 hours anneal favouring the 48 to 43° C. temperature band. Details of the temperature ramp for the annealing reaction are below in Table 1.

TABLE 1
Temperature ramp for synthesis of DNA origami

| Temp (° C.) | Time |
|---|---|
| 65 | 15 min |
| 65-48 | 1 min/−0.1° C. |
| 48-43 | 15 min/−0.1° C. |
| 43-25 | 2 min/−0.1° C. |
| RT | hold |

### Agarose Gel Electrophoresis

10 μL of 5 nM sample (DNA origami or M13mp18 single-stranded template), or a molecular weight standard was mixed with 2 μL of loading dye and loaded onto an agarose gel prepared by dissolving 1.125-3 grams of agarose in 150 mL of buffer, either with or without 7.5 μL of RedSafe dye. Electrophoresis was performed for 3 hours at 70V in pre-chilled 1×TAE buffer with 6 mM MgCl₂. The gels were then scanned using Alexa Fluorophore 647/488 if the sample was labelled with Alexa Fluorophore or with RedSafe dye if RedSafe was added to the gel. After staining with SYBRgold, the gels were scanned again using SYBRgold.

### DNA Origami Purification

The inventors purified the DNA origami either through extraction of purified sample from an agarose electrophoretic gel or by poly-ethylene glycol (PEG) precipitation.

### Purification by Gel Extraction

A 0.75% agarose gel was prepared by dissolving 1.125 g of agarose in 150 mL of 1×TAE buffer containing 11 mM MgCl₂, with RedSafe staining added for visualization. Samples mixed with loading dye were loaded onto the gel and electrophoresis was carried out for 2.5 hours at 70V on ice. After electrophoresis, DNA origami bands were cut out using a clean blade and transferred to a 500 μL Eppendorf tube, which was centrifuged at maximum speed for 5 minutes to crush the gel. The gel slurry was then transferred to a Freeze ‘N’ Squeeze column and centrifuged at maximum rcf for 10 minutes at 4° C. Finally, the purified DNA origami was collected in the collection tube provided with the column.

### Purification by Poly-Ethylene Glycol Precipitation (PEG)

Unpurified DNA origami samples were pipetted into a DNA-lowbind tube and made it up to 400 μL with pre-PEG buffer (1× origami buffer and 20 mM MgCl₂, 0.45 μm syringe filtered). 400 μl of 2×PEG precipitation buffer (15% (g/mL) PEG8000, 10 mM Tris pH 8, 1 mM EDTA, 500 mM NaCl) was added and the samples were centrifuged at 21100 RCF for 25 min at 20° C. Immediately after centrifugation, the supernatant was removed and resuspended in 1× origami buffer with 6 mM MgCl₂ (also 0.45p m syringe filtered). Nanodrop was used to quantify concentration after resuspending in 50 μL 1× origami buffer with 6 mM MgCl₂. Concentration in nM is concentration in ng/μl divided by 4.8.

### Determination of the Dimensions of the DNA Origami

RELION was used to perform the single particle averaging (SPA) to determine the dimensions of NanoSpanno accurately. All the images used for SPA were collected using the Tecnai microscope at a consistent magnification of 19,000-38,000×.

### Preparation of Grids

Carbon/formvar grids were glow discharged and a droplet of 2% uranyl acetate (or uranyl formate) stain placed onto a piece of parafilm. A 5 μL droplet of 1 nM sample (undiluted from gel purification) was applied to dark shiny side of grid and immediately wicked away from grid using a piece of filter paper touched to the edge of the grid. The grid was then touched (dark shiny side) to the droplet of stain, which was immediately wicked away with a piece of filter paper. The grid was then air dried for several minutes.

### Imaging Conditions

All the images used for SPA were collected using the Tecnai microscope at a consistent magnification of 19,000-38,000×.

### 2D Particle Averaging

RELION was used to perform the single particle averaging (SPA) to determine the dimensions of NanoSpanno accurately.

### Preparation of Grids for Cryo-TEM

To prepare the sample for observation in a cryo-electron microscope, an aqueous solution was applied onto a glow-discharged (hydrophilic) holey carbon film, supported by an EM grid. Excess solution is carefully removed using filter paper from one or both sides. The blotted grid is then quickly plunged into a cryogen that has been pre-cooled to liquid nitrogen temperature. This rapid freezing process embeds the biomolecules in a thin, amorphous ice film, allowing for their observation in the cryo-electron microscope.

Imaging with Cryo-TEM

The frozen grid is mounted on a cryo-transfer holder equipped with liquid nitrogen Dewar in a cryo-workstation to avoid ice contamination. Then, the ice-embedded specimen is loaded in frozen state contamination-free into the cryo electron microscope by means of a cryo-transfer holder. Images of holes are taken using low dose mode. Focus is adjusted and then an exposure of the target is acquired at the desired preset magnification.

Coating of Spherical Gold Nanoparticles with DNA
Materials Used for Coating Gold Nanoparticles with DNA
100 nm gold nanoparticles (AuNPs) purchased from Nanopartz
Thiolated DNA purchased from IDT (Coralville USA)
phosphate buffer [prepared: 980 μL MQ water mixed with 10 μl Tween 20 (10%), 10 μl of a potassium phosphate (4:5 mixture of 1M KH₂PO₄ and 1M K₂HPO₄)]
10% Tween-20
20 mM TCEP: 5.732 mg/mL (in MQW, adjust pH to 3.0)

### Preparation of Thiolated DNA

25 μM Thiol-DNA in 10 mM TCEP was prepared by adding 0.5 μL Monothiol-DNA (25AC, 500 μM in Milli-Q water)+5 μL TCEP (20 mM, pH=3)+4.5 μL Milli-Q water. This solution was incubated at room temperature for 1 hour. Then 25 μL Milli-Q water was added to yield a total volume of 35 μL.

Coating of Gold Nanoparticles with Thiolated DNA

1 mL of nanoparticle solution (10 μM, added 10 μL 5× phosphate buffer) was centrifuged at 4500 RCF for 5 min. The supernatant was pipetted off and the particle pellet was mixed with 35 μL TCEP treated DNA and then diluted in the 10 μL 5× phosphate buffer and mixed (total 50 μL). Afterwards, the nanoparticle-oligonucleotide mixture was frozen at −20° C. for 2 h, and then put at RT. The final conditions were consisted of gold nanoparticles at a concentration of 0.2 nM and DNA at 5 μM-25,000-fold molar excess.

Excess DNA was removed from DNA-coated gold nanoparticles by repeated sedimentation and removal of supernatant. The mixture was spun down at 4500 RCF for 5 min and the supernatant was pipetted off and the particle pellet diluted in 10 μL 5× phosphate buffer and then in 1 mL MQW. The sedimentation process was repeated 5 times to completely purify nanoparticles from free oligonucleotides.

### Protein Expression and Purification

### Materials

1000× ampicillin: 100 mg/ml (1 g/10 mL)
LB Agar
LB Broth
1000×IPTG: 1M (0.238 g/mL)
Klenow Buffer: 10 mM Tris HCl pH 8, 50 mM NaCl, 10 mM MgCl₂, and 1 mM DTT
Phi29 Buffer: 50 mM Tris HCl pH 7.5, 50 mM NaCl, 10 mM MgCl₂, and 4 mM DTT
Taq: 10 mM Tris HCl pH 8, 50 mM NaCl, 1.5 mM MgCl₂
120 mL 1M imidazole
500 mL Lysis Buffer: 20 mM Tris pH 7.5, 300 mM NaCl (420 mL for wash buffer)
500 ml Wash Buffer: 20 mM Tris pH 7.5, 300 mM NaCl, 20 mM imidazole
200 mL Elution Buffer: 20 mM Tris pH 7.5, 300 mM NaCl, 500 mM imidazole
1 L SEC Buffer: 20 mM Tris pH 7.5, 150 mM NaCl, 1 mM DTT
100 mL SEC Buffer (post fluorophore labelling): 20 mM Tris pH 7.5, 150 mM NaCl
Dialysis Buffer: 20 mM Tris pH 7.5, 150 mM NaCl
0.1M TCEP pH 7
Alexa647 fluorophore in 50 nmole aliquots (resuspend in 10 uL DMSO for stock concentration of 5 mM)

### Description of Plasmids

Three petDuet-1 plasmids each containing one of the recombinant polymerases; Klenow-SpyC, Phi29-SpyC, and Taq-SpyC were ordered (Genscript). Each recombinant polymerase construct was cloned into the petDuet-1 vector for expression in E. coli cells.

Transformation of T7 E. coli with Expression Plasmids

LB agar plates with ampicillin were prepared by heating 300 ml Luria Broth (LB) agar to liquid and cooling to room temperature. Prior to solidifying 300 μL 100 mg/ml ampicillin was added to LB agar to yield a final concentration of 100 μg/ml ampicillin. LB agar was then poured into sterilised Petri dishes and allowed to solidify.

1 μL of plasmid transformed into 20 μL T7 E. coli expression cells. Cells were first incubated on ice for 30 min and 1 μl of expression plasmid added. The mixture was then incubated for 10 s in a water bath at 42° C. then placed back on ice for 5 minutes. 20 μl of transformed cells were spread onto Luria Broth (LB) agar plates containing 100 μg/ml ampicillin and incubated overnight at 37° C. Single colonies containing expression plasmids were observed.

### Protein Expression

A single E. coli colony was placed into a 100 ml starter culture containing 100 μg/ml ampicillin and incubated overnight at 37° C.~25 mL starter culture was added to 2 L LB broth containing 100 μg/ml ampicillin. Cultures were grown at 37° C. on a shaking platform rotating 180 rpm until an OD600 of 0.5-0.6 was reached. The temperature was then reduced to 18° C., and IPTG was added to a final concentration of 1 mM to induce protein expression. Cells were then left to produce protein overnight on an orbital shaker, which was rotating at 180 rpm.

Cells were then centrifuged at 6000 RCF at 4° C. for 20 min (centrifuge VX22N, rotor R9A2, VWR). Cell pellets could be stored at −80° C. for later use. Cell pellets were resuspended into 80 mL lysis buffer (+1 Complete EDTA tablet+DNAse 1) and sonicated 3×3 min with a 50% duty cycle, power output of 7, 3 to lyse the cells. Protein expressed in the cytoplasm was extracted by removing solid material by centrifugation. Samples were centrifuged at 3,600 RCF for 30 min at 4° C. The pellet was discarded and cell lysate supernatant collected for purification (centrifuge VX22N, rotor R9A2, VWR).

### Protein Purification by Immobilized Metal Affinity Purification (IMAC)

Immobilized metal affinity chromatography (IMAC) was performed using a 2×5 mL HisTrap Fast Flow Crude column. The HisTrap column was prepared by first equilibrating with Wash Buffer (30 mL, 3× column volume), followed by 30 ml of elution buffer to remove any contaminants, the equilibrated again with 30 ml wash buffer prior to loading cell lysate.

Around 75 ml cell lysate was filtered with a 0.22 μm membrane then loaded onto the HisTrap column at a flow rate of 1 ml/min. The HisTrap column was then washed with Wash Buffer (20 mL) prior to eluting the bound protein. A gradient elution was performed over 10 column volumes from 20 mM to 500 mM imidazole at 2 ml/min and 2 ml fractions collected.

### Protein Purification by Size Exclusion Chromatography (SEC)

Elution fractions containing the recombinant polymerase enzymes from IMAC purification were combined and concentrated to 5 mL with 30 KD Amicon Ultra Centrifugal Filter Unit and further purified with SEC. SEC was performed using a HiLoad 16/600 Superdex 200 pg column (GE Healthcare). The column was equilibrated with MQW (~150 mL, 1.2× column volume, 0.9 ml/min, less than 0.5 Mpa). The SEC column was equilibrated with SEC Buffer (~150 mL, 1.2× column volume, 1 ml/min, less than 0.5 Mpa). Proteins were eluted from the SEC column using SEC Buffer (1.2× column volume, 150 mL, inject less than 5 mL). Elution fractions were run on SDS-PAGE gels as described above to check for protein purity. Fractions containing pure polymerase were pooled and the final concentration was determined by Denovix. Recombinant polymerases were stored in at −80° C. until required.

### Covalent Attachment of Fluorescent Dyes to Protein

Purified protein was dialysed to remove DTT. 1 mL of protein at ~80 μM were added to dialysis buttons with 6 kD cutoff snakeskin. Buttons were placed into 1 L dialysis buffer, stirring constantly at 4° C. overnight with one buffer change. Following dialysis, the concentration was determined via IR absorption on the Denovix instrument.

400 μL of dialysed protein at 50 μM, 10 ul TCEP (20 mM, 10 μL) was added to 400 μl of 50 μM dialysed protein and left on ice for ~30 min. Proteins were then labelled with 5 times molar excess of Alexa-647 maleimide dye. 400 μl of the protein with TCEP was added to a 20 μl solution of DMSO containing 100 nmole of Alexa-647 dye (5× excess). The mixture was incubated in the dark on a rotating wheel at, overnight. Excess dye was then purified and buffer exchanged into 20 mM Tris pH 7.5, 150 mM NaCl with SEC.

### SDS-PAGE

4-12% SDS-PAGE gels were purchased from Invitrogen. Samples were run at 180V for 30 min in 1×MES buffer. The protein molecular weight (MW) standard used in all SDS PAGE gels was the SeeBlue Plus2 Pre-Stained Protein Standard. Gels were post-stained with Blue and imaged under UV light to identify which fractions contained pure protein.

### High Performance Liquid Chromatography (HPLC)

The HPLC purification of maleimide DNA, DNA-peptide conjugates, and fluorophore labelled dNTPs was performed using a Shimadzu LC-20AT HPLC system equipped with an Atlantis RP-HPLC C18 column (150 mm×4.6 mm, 5 m). Fractions were automatically collected, and those containing pure product were subjected to evaporation. The resulting residue was dissolved in water, and the quantification was carried out using a Denovix instrument.

### HPLC Method for Maleimide DNA and DNA-Peptide Conjugates

The samples were diluted with 0.1M TEAA+5% ACN and a volume of 100 μL was injected into the HPLC. The HPLC buffers include HPLC buffer A, which contains 0.1M TEAA+5% ACN, and HPLC buffer B, which consists of 0.1M TEAA+70% ACN. The HPLC procedure is conducted by injecting the sample at t=0 min and flowing buffers at a rate of 1 mL/min. The schedule includes pumping B.Conc 0% for 1 minute, then increasing the concentration to B.Conc 100% from 1 to 40 minutes and maintaining it at B.Conc 100% until 43 minutes. The concentration is then reduced to B.Conc 0 at 43.01 minutes and maintained until 48 minutes.

HPLC Method for Fluorophore Labelled dNTPs

Samples were diluted with 0.1M TEAA and a volume of 100 μL was injected. The buffers used in the HPLC method are HPLC buffer A, which consists of 0.1M TEAA, and HPLC buffer B, which consists of 100% ACN. The procedure involves running the HPLC method, where the sample is injected at t=0 min, and the buffers are flowed at 1 mL/min according to a specific schedule. The schedule involves pumping B.Conc 2% for 1 minute, then increasing the concentration to B.Conc 7% from 1 to 15 minutes, then to B.Conc 30% from 15 to 45 minutes, and finally to B.Conc 60% from 45 to 75 min and maintain at 60% until 80 minutes. After 80.01 minutes, the concentration is reduced back to B.Conc 2% and maintained for 5.00 minutes until the controller stops at 85.01 minutes.

### Synthesis of DNA-Peptide Conjugate

### Synthesis and Purification of Maleimide-DNA

To synthesise maleimide-DNA, 2 mg SMCC was resuspended in 300 μL DMF and 10 μL DIPEA added. Then 5 μL of 2 mM aminated DNA (10 nmol) was added to the solution, which was incubated on an orbital shaker for 45 minutes at room temperature at 600 rpm.

Next, SMCC-DNA was extracted by ethanol precipitation. The solution was split into 3×~105 μL aliquots and 100 μL MilliQ water, 20 μL of 3M sodium acetate pH 5.2 and 800 μL ice-cold 100% EtOH added to each tube and incubated at −80 deg for 45 min. DNA precipitate was pelleted by centrifugation at 25000 RCF for 45 minutes at 4° C. and supernatant removed. The pellet was dried (~15 min in the oven at 37° C.) and stored at −20° C.

A final purification step was performed with high performance liquid chromatography (HPLC). The pellet was resuspended in 100 μL MilliQ water, and undissolved precipitate removed by centrifugation and filtration. The supernatant was loaded onto HPLC using Method 2.10.1 and eluted with a gradient of acetonitrile. Typically, 5′-Maleimide-15 nt DNA and 3′-Maleimide-15 nt DNA elute at approximately 15 minutes, while 5′Cy5-3′Maleimide-15 nt DNA elutes at around 22 minutes. Fractions containing pure product were evaporated, and the remaining residue was dissolved in water and quantitated using UV-Vis spectrophotometry. The product was then divided into 100 pmol/tube, allowing for resuspension in 10 μL of MQW to create a 10 μM working stock. The samples were then dried using speedyvac and stored at −20° C.

### Conjugation of Maleimide DNA to Peptide Conjugate

Maleimide-DNA was resuspended in 80 μl MilliQ water and 10 μL 200 mM Tris pH 7.5, 1.5M NaCl. Then 10 μl of 1 mM cysteine containing peptide was added and the mixture incubated at 2 h at room temperature before purification with HPLC. The 100 μL sample was loaded on HPLC as described in section of Example 1 headed “HPLC method for maleimide DNA and DNA-peptide conjugates”.

Typically, 5′-SpyTag-15 nt DNA and 3′-SpyTag-15 nt DNA elute at approximately 17 minutes, while 5′Cy5-3′Maleimide-15 nt DNA elutes at around 20 minutes. Fractions containing pure product were evaporated, and the remaining residue was dissolved in water and quantitated using UV-Vis spectrophotometry. The product was then divided into 100 pmol/tube, allowing for resuspension in 10 μL of MQW to create a 10 μM working stock. The samples were then dried using speedyvac and stored at −20° C.

### Synthesis of DNA-Protein Conjugate

Purified SpyTag-DNA could then be bound to purified polymerase-SpyCatcher to yield polymerase enzymes covalently linked to a DNA strand with the desired sequence to enable specific immobilisation in the cavity of the DNA origami nanoantenna.

Buffer conditions are below:
Klenow 10× reaction buffer: 100 mM Tris pH 8, 0.5 M NaCl, 100 mM MgCl2¬¬, 10 mM DTT
Binding and elution buffer:
   1M Imidazole solution (68 mg/mL)
   Binding and wash buffer: 20 mM Tris pH 7.5, 300 mM NaCl, 10 mM imidazole
   Elution buffer: 20 mM Tris pH 7.5, 300 mM NaCl, 500 mM imidazole

A 2-hour RT incubation of SpyTag-DNA with SpyCatcher-proteins was carried out, followed by a 70 uL reaction with 2 times molar excess of SpyTag-DNA over SpyCatcher-proteins. The reaction was performed in the respective reaction buffers for SpyCatcher-proteins. Excess SpyTag-DNA was removed by purification of protein with IMAC. The mixture was diluted 1 in 5 (final volume of reaction=350 μl) by adding 280 uL binding buffer. Tris Nickel-NTA (NiNTA) beads were prepared as follows: supernatant was removed from 100 μl bead slurry and wash 3 times with 300 μL MilliQ water (centrifuge speed and time: 500×g for 30 s). The beads were then equilibrated with 2×300 μl exchanges with binding & washing buffer. The solution containing SpyTag-DNA and polymerase-SpyCatcher were added to the NiNTA beads and incubated for 2 hours at 4° C. on a rotating platform. Supernatent was removed by centrifugation (500×g for 30 s) and beads washed three times with 300 μl wash buffer (centrifuge speed and time: 500×g for 30 s) to remove excess SpyTag-DNA. Polymerase-DNA was recovered from the supernatent NiNTA beads after incubation with 70 μl elution buffer for 2 hours at 4° C. on rotator. The sample was then buffer exchanged into the desired buffer for assembly onto DNA origami scaffolds with desalting spin SEC column and sample purity assessed with SDS PAGE where protein was visualised with a protein-specific stain and by the fluorescence signal from the covalently attached fluorophore.

### DNA Polymerase Activity Assays

DNA polymerase function was assessed by measuring the extension of a fluorescently labelled primer bound to the target DNA strand. Reaction conditions are summarised below:

| | |
|---|---|
| Fluorescent Primer | 1 μM |
| Template | 1 μM |
| Polymerase | 200 nM |
| dNTPs | 100 μM |
| Klenow | 10 mM Tris HCl pH 8, 50 mM NaCl, 10 mM |
| | MgCl₂, and 1 mM DTT |
| Phi29 | 50 mM Tris HCl pH 7.5, 50 mM NaCl, 10 mM |
| | MgCl₂, and 4 mM DTT |
| Taq | 10 mM Tris HCl pH 8, 50 mM NaCl, 1.5 mM |
| | MgCl₂ |

Reactions were stopped by the addition of 0.5 μl of 0.5 M EDTA followed by heat denaturation at 95° C. for 15 minutes. Extension of primers by DNA polymerase were visualised by native PAGE.

### Native PAGE

15% Tris-Glycine Native-Polyacrylamide Gel (with 5% stacking gel) run for 10 min at 150V, 45 min at 250V (until dye reaches 1.5 cm from bottom).

### Assembly of Gold Nanoparticles on DNA Origami Scaffold

DNA-coated gold nanoparticles were resuspended in 1×TAE containing 6 mM MgCl₂ and mixed with 500 μM of purified DNA origami with single-stranded DNA extensions that were complementary to thiolated DNA bound to the gold nanoparticles.

### Assembly of DNA Polymerase Enzymes on DNA Origami Scaffold

100 nM polymerase-DNA conjugate and 20 nM DNA origami scaffold were incubated in a 10 uL reaction volume at room temperature for 2 hours. The reaction buffer is 10 mM Tris HCl pH 8, 50 mM NaCl, 10 mM MgCl₂ Excess polymerase-DNA was removed with SEC as follows:
Prepared S300 columns, containing 500 μL slurry for each complex
Spun resin 1×2 min at 1000 g
Washed resin with 4×500 μL MQW, 1 min at 1000 g
Washed resin with 3×500 μL imaging buffer, 1.5 min at 1000 g
Spun resin for 1.5 min at 1000 g
Loaded ~10 μL onto 1×5300 column
Spun 4 min at 1000 g
   Nanodrop concentration in nM is concentration in ng/μl divided by 4.8

### Surface Plasmon Resonance (SPR)

Experiments were performed on a Biacore S200 instrument (GE Healthcare Life Sciences). All experiments were performed at room temperature using Klenow reaction buffer and a flow rate of 10 L/min. CM3 sensor chips were coupled with streptavidin to near saturation (typically between 4000 and 7000 RU) using the amine coupling kit (GE Healthcare Life Sciences). After streptavidin was coupled, biotinylated DNA strands were injected into reference and experimental flow cells before excess biotin-binding sites were blocked with biotin in Klenow reaction buffer. Experimental flow cells were then loaded with DNA template strands. Experiments were performed with 2 flow cells on a chip, with the remaining flow cells being a reference cell with no DNA template. The surface was then conditioned with 2 injections of Klenow reaction buffer followed by the indicated concentrations of DNA polymerase. Reference-subtracted data are shown, and all data were fit in MATLAB 2020b.

### Single Molecule Total Internal Reflection Fluorescence (TIRF) Microscopy

### General Setup

Microfluidic devices and coverslips functionalised with BSA-biotin and streptavidin were prepared as described previously25. Samples were added to each channel at a concentration of 10 pM in imaging buffer, before washing with imaging buffer. Images were collected on a custom built TIRF microscope described in McGuinness et al²⁶. with a power density of ~1-3 W cm⁻² (measured at the objective with the laser beam normal to the surface of the coverslip).

### Single Particle Photobleaching

Single particle photobleaching was performed using purified, preassembled DNA origami scaffold and DNA polymerase in DNA origami imaging buffer immobilized on a coverslip to a density of ~1000 particles per field of view (FOV). Images were acquired with 488 (50 mW) and 647 nm excitation (20 mW) with a 200 ms exposure time and 200 frames per FOV. Three independent repeats of 20 FOV were obtained. Images were analyzed with the JIM-Immobilized-Microscopy-Suite (https://github.com/lilbutsa/JIM-Immobilized-Microscopy-Suite) to determine single photobleaching steps and their corresponding step heights.

In order to measure the enhancement of fluorescence in the presence of metallic nanoparticles, single-particle photobleaching was performed using purified, preassembled DNA origami scaffold with an Alexa-647 fluorophore in the hotspot region (FIG. 1) which was then incubated at a concentration of 100 pM with 500 pM DNA-coated 100 nm AuNPs in DNA origami imaging buffer for 2 hours before immobilization on a coverslip. Image acquisition was performed as described above.

### Transient Binding of Fluorescent DNA Strands to Plasmonic Nanoantennas

To measure the enhancement of fluorescence of multiple individual fluorophores over time, a docking strand containing a 7 base pair ssDNA binding site (TCCTCCT)27 was incorporated into the DNA origami scaffold in the hotspot region. DNA origami scaffolds were purified, assembled with AuNPs and immobilised on a coverslip as described above. 1 nM imaging strand (Alexa647-AGGAGGA) was then added before imaging for 5000 frames with an exposure time of 200 ms and a 639-laser power of 20 mW.

### Measurement of Gold Scattering Intensity Vs Polarisation Angle

Variation of incident polarisation was achieved through the use of a polarising filter followed by a zero-order half-waveplate in the excitation light path which was rotated in 100 increments to achieve a variation of polarisation angle in 20° increments. Measurements were obtained using an exposure time of is and a 639-laser power of 20 mW.

### DNA Sequencing Measurements

For sequencing measurements, assembled nanoantennas were loaded onto coverslips as described above, with a subsequent step in which 1 μM biotinylated DNA complementary to the DNA coating the AuNPs was added for 1 min in order to immobilise the AuNPs on the coverslip before washing with imaging buffer. Photobleaching measurements were then performed as described to localise correctly assembled particles, before the initiation of the sequencing measurement using a 639 and 568 laser at 20 mW laser power, with an exposure time of 100 ms for 20,000 frames. Once this measurement was initiated, 100 nM dNTPs were flowed into the channel at 50 μl/min for 2 minutes before the flow rate was reduced to 10 μl/min.

Synthesis of Fluorescently Labelled dNTPs

### Materials

Aminated dNTP: γ-(6-Aminohexyl)-dGTP, γ-(6-Aminohexyl)-dATP, γ-(6-Aminohexyl)-dCTP, γ-(6-Aminohexyl)-dTTP, all 50 μl (10 mM) stored at −20° C.
Alexa Fluor™ 647 NHS Ester: fresh in DMSO, 25 nmole/tube, speedyvac'd to dry
Alexa Fluor™ 568 NHS Ester: fresh in DMSO, 25 nmole/tube, speedyvac'd to dry
10× Reaction buffer: 1M NaHCO₃ pH=8.30 (fresh)
HPLC buffer A: 0.1M TEAA
HPLC buffer B: Acetonitrile

### Amine and Ester Crosslinking Reaction

Alexa Fluor™ 568/647 NHS Ester was dissolved in 5 μL DMSO to a stock concentration of 5 mM. 10 μl of 0.4 mM Alexa Fluor™ 568/647 NHS Ester and 1 mM γ-(6-Aminohexyl)-dNTP in reaction buffer were incubated at room temperature for 2 hours before purification with HPLC as above.

### Example 2. Design and Embodiment of Plasmonic Nanoantenna DNA Sequencer

The plasmonic nanoantenna consists of three components. First, metallic nanoparticles (in this case spherical gold nanoparticles with a mean diameter of 100 nm). Second, a DNA polymerase enzyme. Third a nanoscopic scaffold constructed from DNA is used to control the spatial localisation of metallic nanoparticles and DNA polymerase to create a field of electromagnetic enhancement directly at a single DNA polymerase enzyme, which is immobilised in a cavity directly in between the gold nanoparticles (FIG. 1A). To enable sequencing read out, dNTPs are labelled with different colour fluorophores so that their sequential incorporation by DNA polymerase can be detected. The scaffold also enables specific immobilisation of the plasmonic nanoantenna on the surface of a glass coverslip for single-molecule fluorescence imaging (FIG. 1i).

The following sections describe the synthesis of each component and the assembly of all components into a complete nanoantenna DNA sequencer.

### Design and Synthesis of DNA Origami Scaffold

The novel DNA sequencer design requires placing a DNA polymerase directly in the plasmon enhancement hotspot created by two 100 nm AuNPs spaced 30 nm apart. Therefore, a novel three-dimensional DNA origami structure was designed and folded from an M13mp18-derived scaffold strand and complementary staple strands.

### Design of DNA Origami Scaffold

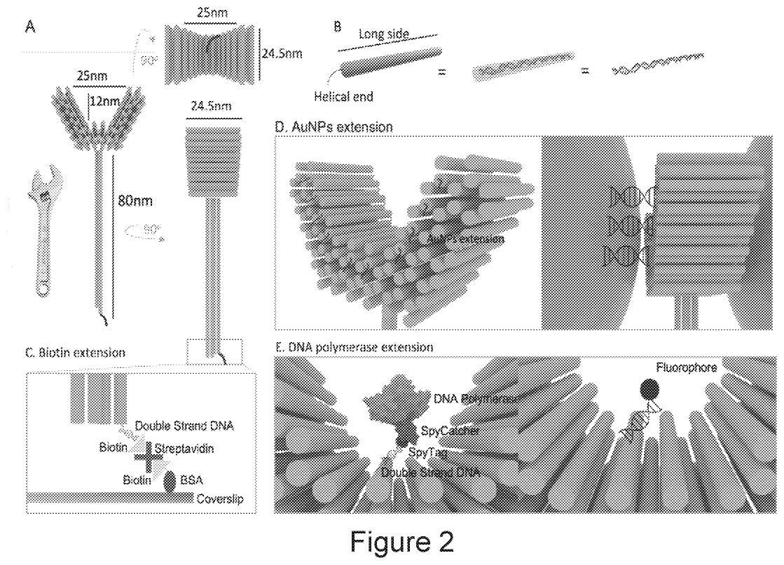

The DNA origami scaffold consists of 86 parallel, interconnected DNA double helices designed into a U-shaped structure with a cavity in the centre using the honeycomb lattice with each helix between the AuNPs set to be 7 turns or 72 bp long (24.5 nm). The cavity has dimensions of approximately 25×12×24.5 nm (FIG. 2A), which should allow space for the polymerases to bind. For immobilization on the coverslip for imaging with fluorescence microscopy, the U-shaped cavity has a long rigid tail approximately 80 nm in length and consisting of a bundle of 4 DNA helices. At the base of the tail are one or more biotin modified DNA staple strands that enable specific immobilisation on BSA-biotin coated coverslips via biotin-streptavidin interactions (FIGS. 2A & C). The overall shape of the DNA origami scaffold resembles that of a nanoscopic spanner, with the U-shaped cavity resembling the spanner head and the tail resembling the spanner handle. This DNA origami scaffold is referred to as the NanoSpanno (NS).

Single-stranded DNA strands can be designed to protrude from DNA helices to create specific binding sites (handles) for proteins or nanoparticles that are decorated with DNA strands that are complementary to these handles (anti-handles). At the spanner head, the 10 DNA staples were extended at the edge of either end of the cavity to provide anchor points for binding DNA-functionalized 100 nm AuNPs (FIGS. 2B & D). With gold nanoparticles bound on either side of the cavity, the estimated gap length is ~35 nm. In the cavity between this gap, another staple extension with an orthogonal DNA sequence is placed as a handle to bind to specifically polymerase enzymes that are covalently bound to a complementary strand of DNA (6E) as detailed in Example 1. This enables the attachment of the polymerase directly in the plasmon enhancement hotspot of a plasmonic nanoantenna (FIG. 5B).

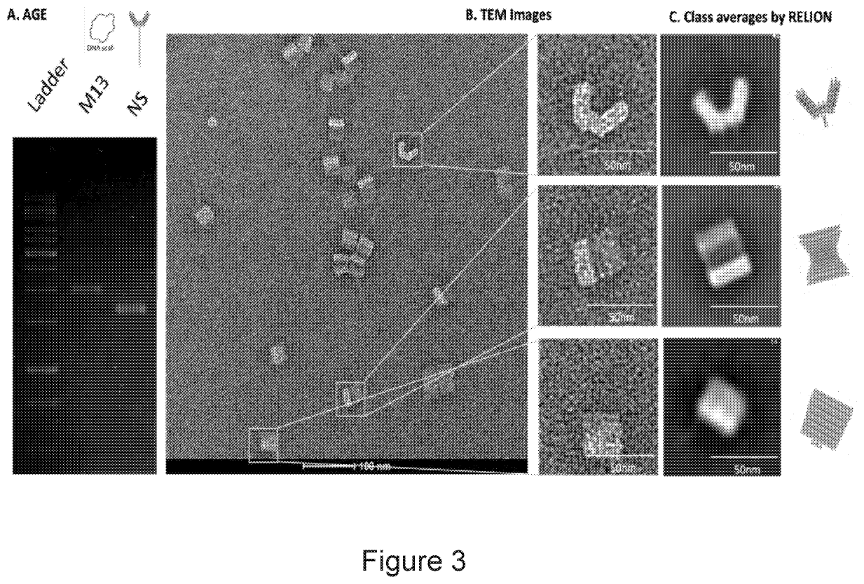

The signal amplification that can be achieved in this NanoSpanno is calculated by incorporating a fluorophore-labelled DNA staple strand (see below) directly into the plasmonic hotspot of the NanoSpanno with a finite element model. These models indicate that fluorescent dyes in the cavity between gold nanoparticles can be observed to be over 3000 times brighter than fluorescent dyes not located in the hotspot. (FIG. 3)

### Synthesis of DNA Origami Scaffold

DNA origami samples were purified analysed on agarose electrophoresis (AGE). Consistent with rigid, stable and uniformly structured DNA origami, the NS migrated at a consistent rate resulting in a single distinct band on agarose electrophoresis. Moreover, the migration rate was faster than the M13mp18 scaffold alone, which is indicative of a more compact structure with a lower radius of gyration (FIG. 3A). Together these observations on AGE are indicative of a well-formed DNA origami structure.

To verify that the DNA origami structure was consistent with design, we directly visualised purified structures with transmission electron microscopy. We observed NS structures in different orientations. An example micrograph showing multiple single particles can be seen in FIG. 3B with 2D class averages from multiple particles in different orientations are shown in FIG. 3C. DNA origami particles appeared to be consistently well formed with shapes and dimensions that were consistent with design, thus verifying their structural integrity.

### Preparation of DNA-Coated Gold Nanoparticles

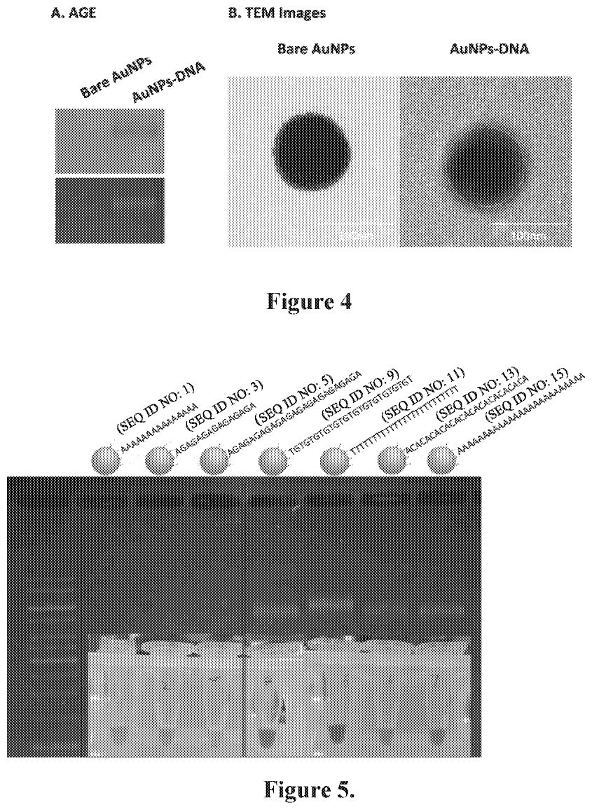

Binding of metallic nanoparticles to specific sites on the DNA origami scaffold was facilitated by DNA hybridisation. Single-stranded DNA on either side of the cavity were designed to be complimentary to single-stranded DNA that were used to coat gold nanoparticles with thiol-gold chemistry²⁸. The optimised method for coating metallic nanoparticles is described herein. Agarose gel electrophoresis demonstrates co-localisation of thiolated DNA with gold nanoparticles (FIG. 4A) and DNA-coated gold nanoparticles also resulted in a light halo on the surface of gold nanoparticles visualised in TEM. This light halo was not observable in uncoated gold nanoparticles (FIG. 4B). Combined these data are consistent with effective coating of gold nanoparticles with thiolated DNA.

Since 100 nm gold nanoparticles tended to aggregate easily, the method for coating nanoparticles with high efficiency was critical. Much optimisation was required to determine the correct length and sequence of these single-stranded DNA to ensure that they achieved the following specifications:
1. Did not cause aggregation of DNA origami structures
2. Were able to prevent aggregation of gold nanoparticles. This was particularly challenging for nanoparticles with 100 nm diameter
3. Could efficiently enable specific coupling of pairs of nanoparticles to the specific binding site on the DNA origami scaffold

Table 2 below details the different DNA strands that were bound to gold nanoparticles to test nanoparticle stability, DNA-gold binding efficiency and assembly onto the NS DNA origami scaffold.

TABLE 2
Details of different DNA strands that were bound to gold nanoparticles

| | | | Complementary DNA extensions |
|---|---|---|---|
| DNA bound to 100 nm AuNP (5′-3′) | | | on DNA origami (5′-3′) |
| 1 | 15 nt | Dithol-AAAAAAAAAAAAAAA | TTTTTTTTTTTTTTT (SEQ ID NO: 2) |
| | | (SEQ ID NO: 1) | |
| 2 | 15 nt | Dithiol-AGAGAGAGAGAGAGA | TCTCTCTCTCTCTCT (SEQ ID NO: 4) |
| | | (SEQ ID NO: 3) | |
| 3 | 25 nt | Dithiol- | TCTCTCTCTCTCTCTCTCTCTCTCT |
| | | AGAGAGAGAGAGAGAGAGAGAGAGA | (SEQ ID NO: 6) |
| | | (SEQ ID NO: 5) | |
| 4 | 25 nt | Dithiol- | TGTGTGTGTGTGTGTGTGTGTGTGT |
| | | ACACACACACACACACACACACACA | (SEQ ID NO: 8) |
| | | (SEQ ID NO: 7) | |
| 5 | 25 nt | Dithol- | ACACACACACACACACACACACACA |
| | | TGTGTGTGTGTGTGTGTGTGTGTGT | (SEQ ID NO: 10) |
| | | (SEQ ID NO: 9) | |
| 6 | 25 nt | Dithol- | AAAAAAAAAAAAAAAAAAAAAAAAA |
| | | TTTTTTTTTTTTTTTTTTTTTTTTT | (SEQ ID NO: 12) |
| | | (SEQ ID NO: 11) | |
| 7 | 25 nt | Monothiol- | TGTGTGTGTGTGTGTGTGTGTGTGT |
| | | ACACACACACACACACACACACACA | (SEQ ID NO: 14) |
| | | (SEQ ID NO: 13) | |
| 8 | 25 nt | Monothiol- | TTTTTTTTTTTTTTTTTTTTTTTTT |
| | | AAAAAAAAAAAAAAAAAAAAAAAAA | (SEQ ID NO: 16) |
| | | (SEQ ID NO: 15) | |

### Nanoparticle Stability

After functionalizing the AuNPs with thiolated DNA, samples were carefully resuspending the pellet in 1×TAE buffer containing 6 mM MgCl₂ to ensure that the sample is homogeneously distributed. Stability was defined by the propensity to aggregate in 6 mM MgCl₂, which is sufficiently high to enable binding of gold nanoparticles to DNA origami structures via DNA hybridisation. Aggregation was detected by agarose gel electrophoresis, which yielded distinct DNA-stained bands at a location corresponding to that of gold nanoparticles (FIG. 5), as well as the colour of the solution. Well dispersed colloidal gold forms a clear, red solution whereas aggregated nanoparticles cause the solution to turn purple or clear with visible aggregates in the tube. FIG. 5 illustrates that sequences 5-8 in Table 2, which are all 25 nucleotides in length, once bound to gold nanoparticles yielded stable colloidal solutions, whereas sequences indexed 1-3 resulted in purple or colourless solutions indicating aggregation.

### Design and Synthesis of DNA Polymerase Enzymes

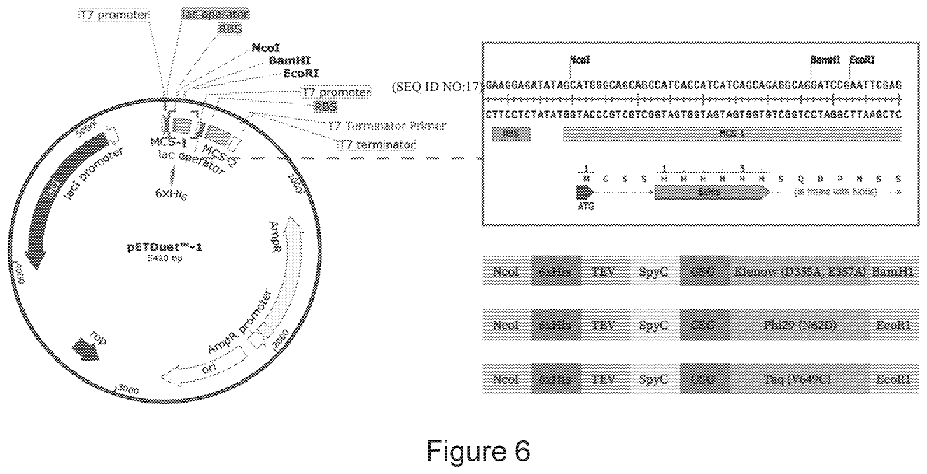

Three polymerases: the DNA polymerase from Thermus aquaticus (Taq), the large (Klenow) fragment of DNA polymerase I from Escherichia coli, and the DNA polymerase from Bacteriophage Phi29 (Phi29) were selected. Each polymerase has different properties, which may be useful for the development of the single molecule sequencer. Recombinant versions of these polymerases were designed such that they were fused at their N terminus to a SpyCatcher domain via a Glycine-Serine-Glycine (GSG) linker, allowing for attachment to a DNA origami scaffold containing a corresponding SpyTag-DNA ‘handle’. N-terminal polyhistidine tags (His-Tag) containing 6× histidine residues (SEQ ID NO: 27) to allow for purification by IMAC were also included. (FIG. 6).

### Purification of Protein Constructs

Proteins were purified with IMAC and SEC (see Example 1) and labelled with AlexFluor 647 (see Example 1) to enable visualisation with fluorescence methods.

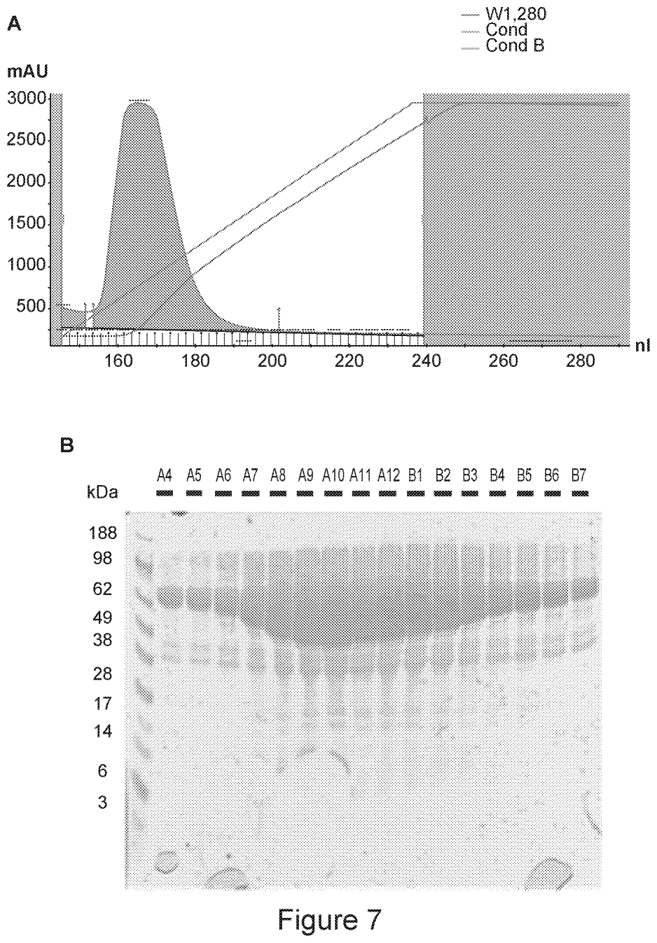

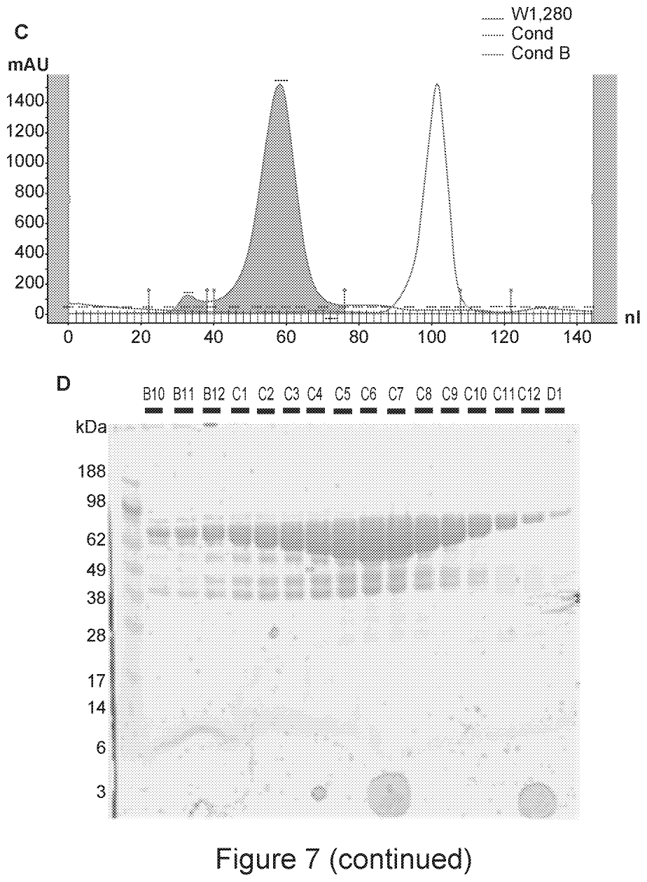

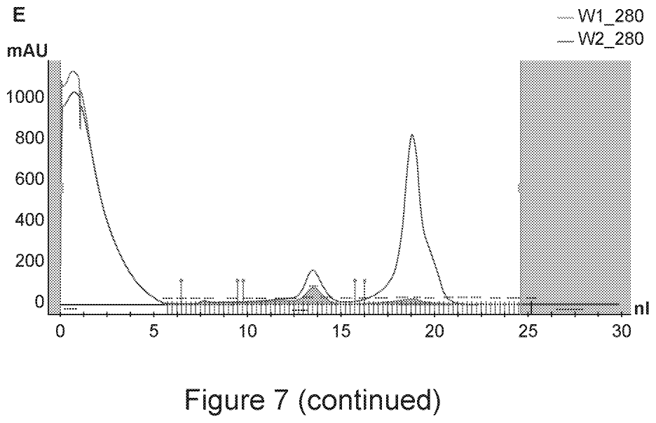

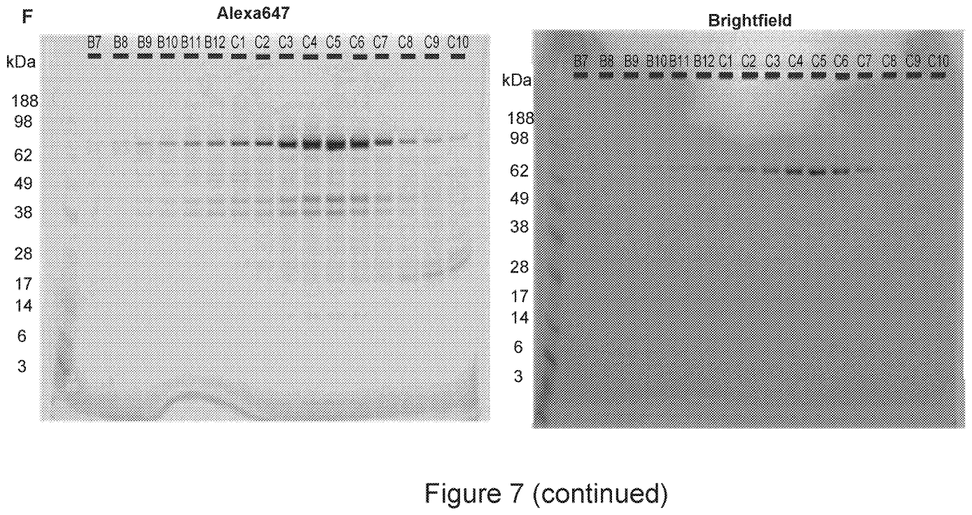

IMAC purification yielded a single elution peak (FIG. 7A) in which fractions yielded a dominant band in SDS PAGE at a molecular weight corresponding to the target protein (FIG. 7B). Peak fractions were combined then further purified with SEC. SEC chromatograms indicate reasonable monodispersity (FIG. 7C) and corresponding SDS PAGE indicate a final purity of >95% (FIG. 7D). The SEC chromatograms indicate Alexa 647 labelling, with a single elution peak visible in both the A280 and A650 measurements (FIG. 7C). Corresponding SDS PAGE analysis shows a dominant band in both the Alexa 647 channel and brightfield after staining (FIG. 7D). The peak fractions were combined, and the absorbance was measured at 280 nm and 650 nm. The results showed that the Alexa 647 labelling efficiency was 100%.

### Synthesis of DNA-SpyTag Peptide Conjugate

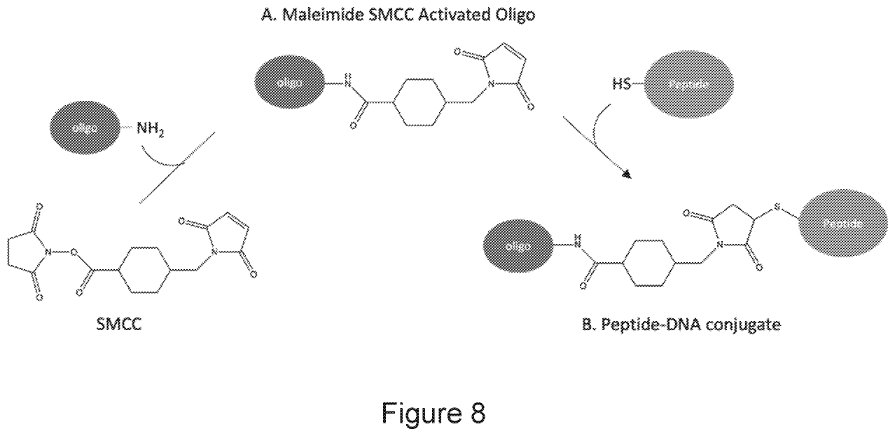

To achieve specific binding to the cavity of the DNA origami scaffold via DNA hybridisation, it was necessary to covalently attach DNA strands to DNA polymerase enzymes. The inventors' approach was to covalently link the SpyTag peptide, which forms an isopeptide bond to the SpyCatcher protein fused to the DNA polymerase enzyme, to a strand of DNA. The DNA-SpyTag conjugation was achieved using a bifunctional crosslinker (SMCC), which consists of a N-hydroxysuccinimide (NHS) ester and a maleimide group. These moieties were bound covalently to a free amino group that was chemically added to the target DNA strand and a sulfhydryl group on a cysteine side chain on the peptide respectively. Thus, yielding the desired DNA-SpyTag construct (FIG. 8).

### Synthesis of Maleimide DNA

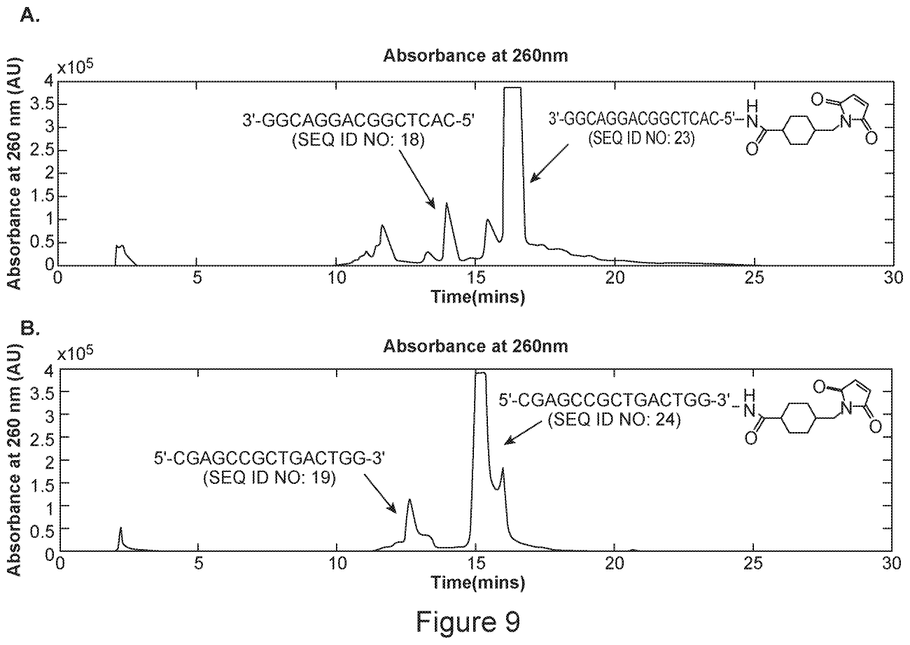

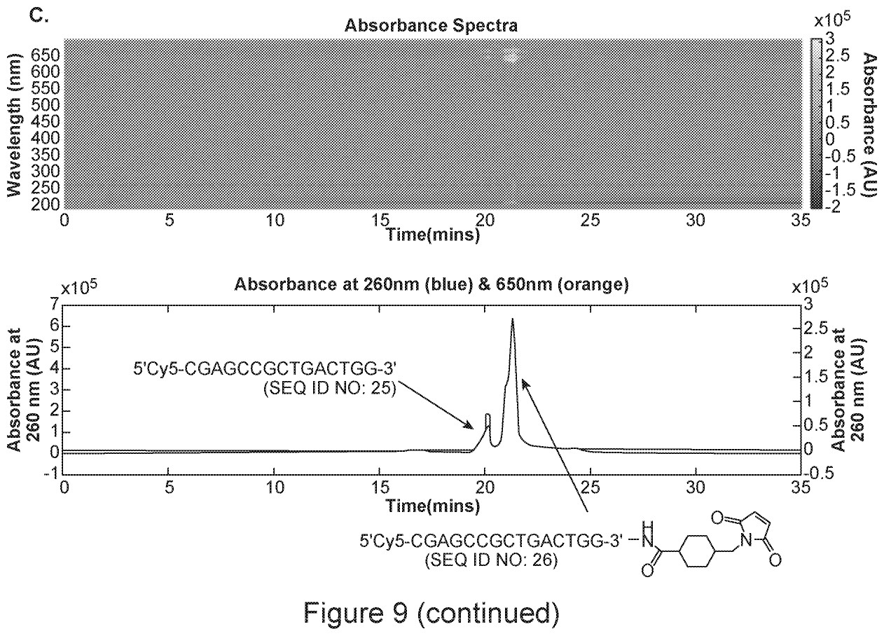

As detailed in Example 1 above, synthesis of DNA-peptide entailed a two-step process. First maleimide DNA was synthesised by reacting SMCC to aminated DNA (FIG. 8A) and purified with ethanol precipitation and HPLC. Maleimide could be selectively appended either the 5′ or 3′ end of the DNA strand. Typical UV chromatograms from HPLC purification are shown in FIGS. 9A and B for 5′ modified and 3′ modified maleimide DNA respectively and FIG. 9C for 5′ Cy5 labelled-3′ maleimide DNA. In both instances, the dominant peak corresponds to maleimide DNA.

### Synthesis of Peptide-DNA Conjugate

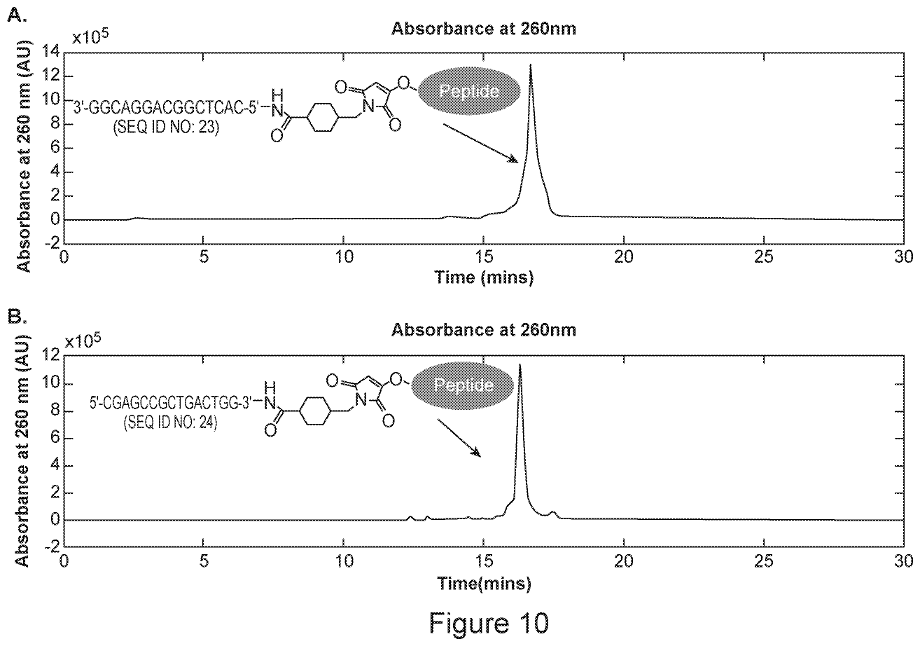

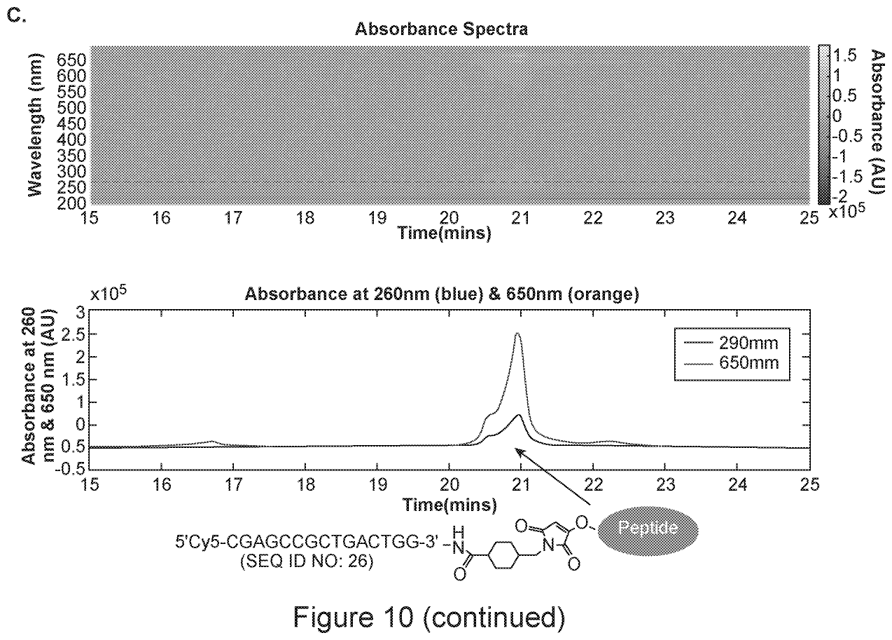

In the second step, maleimide DNA was bound to a free thiol on a cysteine residue on the N-terminus of the target peptide and purified with HPLC (see Example 1). UV chromatograms are shown in FIGS. 10A and B for DNA peptide conjugates where the peptide is appended to the 3′ and 5′ end of the DNA respectively and 5′-Cy5 labelled DNA with peptide appended to the 3′ end in FIG. 10C. In all cases, there was a single-dominant peak corresponding to the DNA-peptide conjugate, which indicates a high purity of the target DNA-peptide conjugate.

### Conjugation of SpyTag-DNA to Polymerase-SpyCatcher

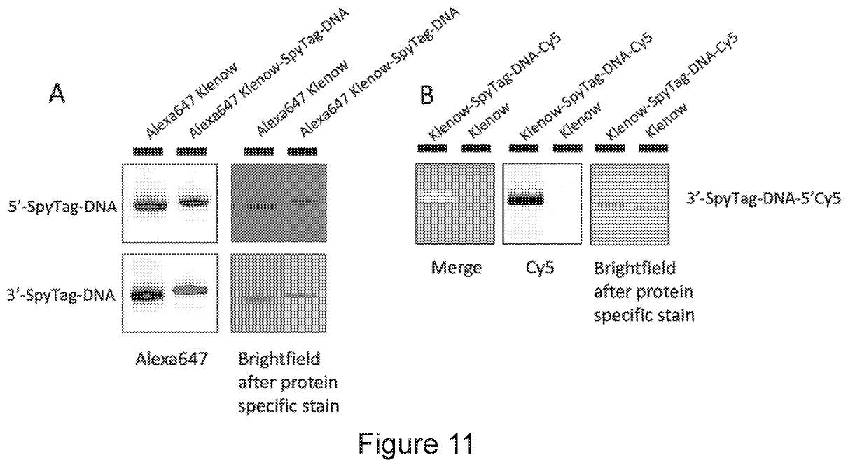

SpyTag-DNA conjugates were bound to polymerase-SpyCatcher fusion protein and purified with IMAC as described in Example 1. Conjugation efficiency was visualised with SDS PAGE, which illustrate a distinctly slower migration rate of DNA polymerase in the presence of SpyTag-DNA compared with DNA polymerase alone (FIG. 11—klenow example). The shift in migration rate occurred irrespective of whether the peptide was bound to the 5′ or 3′ end of the DNA (FIG. 11). A combination of protein-specific stain and fluorescent imaging of gels to specifically localise either fluorescently labelled DNA or protein showed co-localisation of protein and DNA confirming successful assembly of the DNA-labelled polymerase enzymes.

### Demonstration of DNA Labelled DNA Polymerase Function

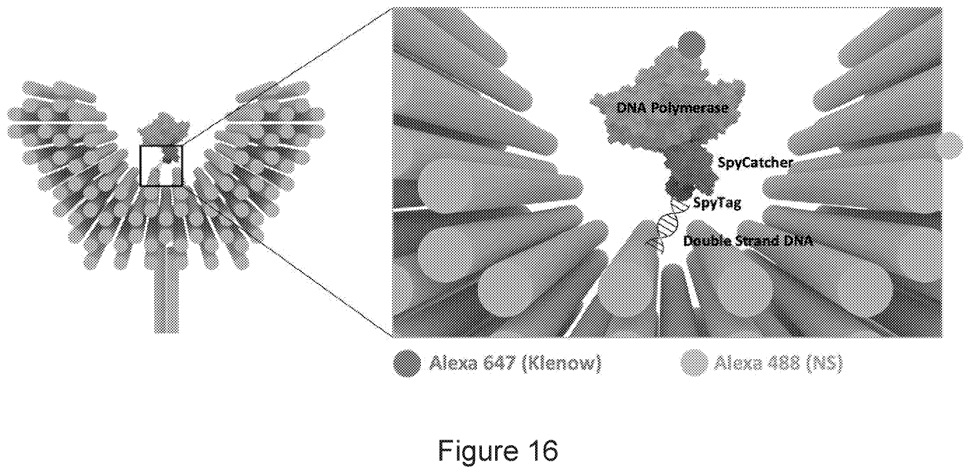

DNA polymerase function was assessed by measuring the extension of a fluorescently labelled primer bound to the target DNA strand. Reactions were stopped at specific time points and primer extensions were visualised by native PAGE. All polymerases appeared to successfully extend the fluorescent primer, resulting in slower migrating bands at from 0.5-1 h compared to 0 h (FIG. 16 A). Both 10 nM and 50 nM Klenow-SpyCatcher-SpyTag-DNA conjugation appeared to successfully extend the fluorescent primer, resulting in slower migrating bands at from 24 h compared to 0 h (FIG. 16B) We conclude that DNA-labelled DNA polymerase enzymes retain their function.

Kinetic Analysis of Polymerase Interactions with Freely Diffusing Primed DNA

It was important to determine the kinetic properties of interactions between DNA polymerase and primed target DNA strands to ensure that we use experimental conditions that enable high efficiency capture of target DNA molecules during sequencing reactions. Since binding kinetics are affected by proximity to surfaces as with single-molecule TIRF microscopy, binding kinetics was measured in bulk with surface plasmon resonance (SPR) (Example 1)

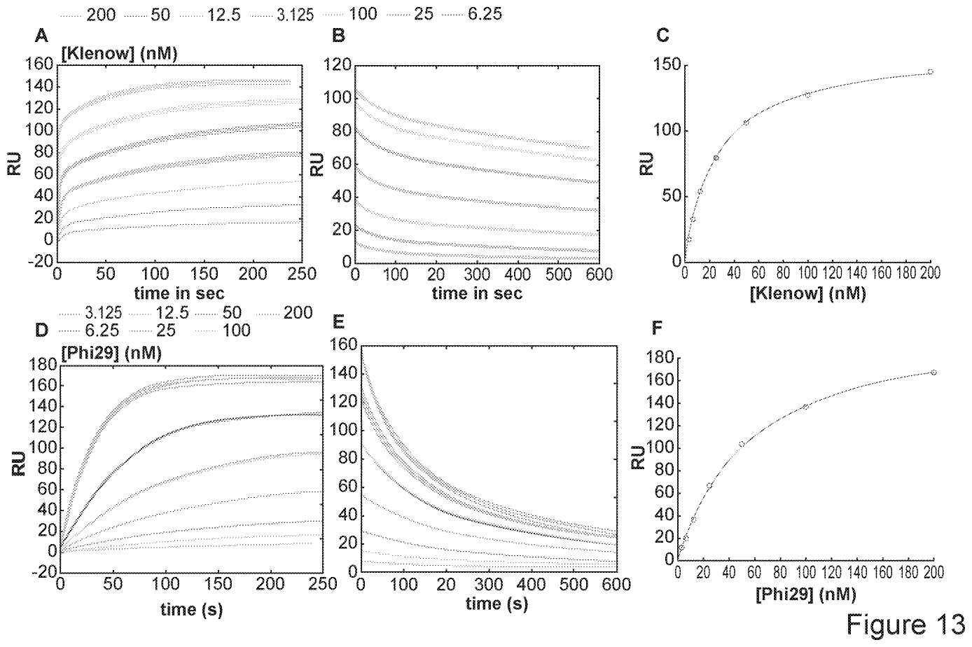

For Klenow, association curves (FIG. 13A) were biphasic and two concentration-dependent association rate constants were extracted. A relatively fast rate constant (k_(a,fast)=3.7±0.3× [symbol] 10 [symbol] \hat{ }6 mol\hat{ }(−1) s\hat{ }(−1)) and a slow rate constant (k_(a,slow)=7.4±0.3× [symbol] 10 [symbol] \hat{ }4 mol\hat{ }(−1) s\hat{ }(−1)). Dissociation curves (FIG. 13B) were also biphasic with a fast dissociation rate constant (k_(d,fast)=1.8±1.1× [symbol] 10 [symbol] \hat{ }(−2) s\hat{ }(−1)) with a mean dissociation time of 38 seconds, and a slow dissociation rate constant (k_(d,slow)=5.9±1.1× [symbol] 10 [symbol] \hat{ }(−4) s\hat{ }(−1)) with a mean dissociation time of 20 minutes. The dissociation rate was determined from the proportion of bound polymerases at steady state (K_D=26±3 nM FIG. 13C).

For Phi29, association curves (FIG. 13D) were also biphasic and two concentration-dependent association rate constants were extracted. Another relatively fast rate constant (k_(a,fast)=1.1±0.3× [symbol] 10 [symbol] \hat{ }6 mol\hat{ }(−1) s\hat{ }(−1)) and a slower rate constant (k_(a,slow)=2.1±0.3× [symbol] 10 [symbol] \hat{ }5 mol\hat{ }(−1) s\hat{ }(−1)). Dissociation curves (FIG. 13E) were also biphasic with a fast dissociation rate constant (k_(d,fast)=1.4±0.1× [symbol] 10 [symbol] \hat{ }(−2) s\hat{ }(−1)) with a mean dissociation time of 50 seconds, and a slower dissociation rate constant (k_(d,slow)=1.2±0.2× [symbol] 10 [symbol] \hat{ }(−3) s\hat{ }(−1)) with a mean dissociation time of 10 minutes. The dissociation constant was determined from the proportion of bound polymerases at steady state (K_D=56±9 nM, FIG. 13F).

### Assembly of Plasmonic Nanoantenna

A major challenge was the determination of sequences and lengths of DNA strands that enabled stable pacification of gold nanoparticles to avoid aggregation in 6 mM MgCl₂, which was required to facilitate binding of gold nanoparticles to DNA origami scaffolds via DNA hybridisation, and that did not result in aggregation of the DNA origami scaffolds themselves. Several combinations of DNA strands and nanoparticle sizes were tested. These combinations and their efficacy at assembling the target structure are summarised in Table 3 below.

TABLE 3
Summary of gold nanoparticle size and the sequences of DNA coating these nanoparticles
that were assembled onto the DNA origami scaffold

| | | | | | Synthesis yield | Synthesis |
|---|---|---|---|---|---|---|
| | | | | | for origami | yield for |
| AuNP | Thiol- | AuNPs | Origami | Free origami | with one | origami with |
| diameter | DNA | aggregation | aggregation | yield | AuNP | two AuNPs |
| 100 nm | 25AC | No | No | 0% | 12.1% | 22.4% |
| | 25TG | No | No | 1.0% | 7.3% | 21.9% |
| | 25T | No | Yes | — | — | — |
| | 25A | No | No | — | — | |
| | 25AG | Yes | No | — | — | — |
| | 15A | Yes | No | — | — | — |
| | 15AG | Yes | No | — | — | — |
| 80 nm | 25AC | No | No | — | — | — |
| 50 nm | 25AC | No | No | 0.7% | 10.9% | 39.5% |

### Assembled Plasmonic Nanoantennas Assessed by Electron Microscopy

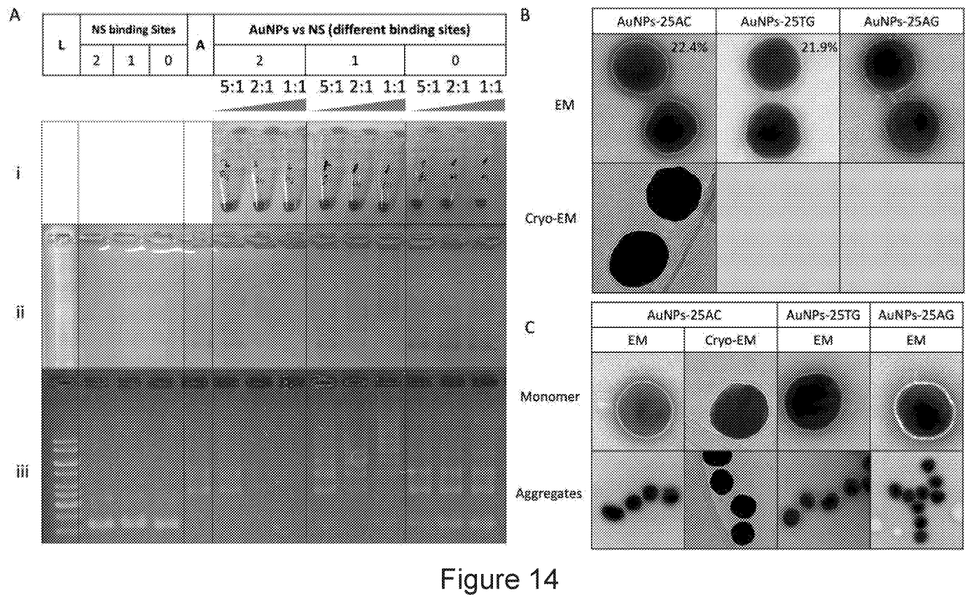

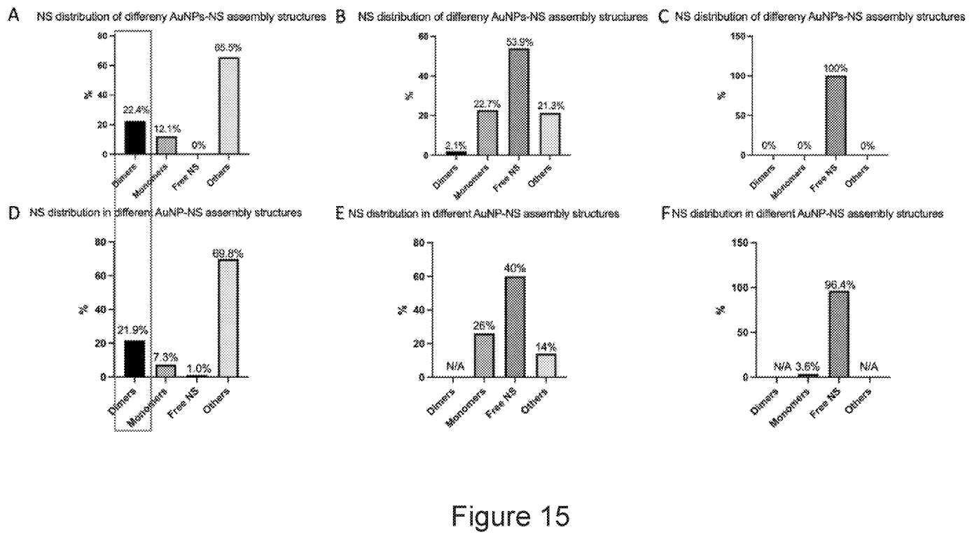

Different molar excesses of gold nanoparticles were tested over DNA origami scaffold and in different configurations of binding sites with 0, 1 or 2 binding sites for gold nanoparticles. After incubation, samples appeared red suggesting that the solution was not dominated by undesirable aggregates (FIG. 14A)

Samples were further analysed with agarose gel electrophoresis. DNA origami alone, configured with 0, 1 or 2 binding sites produced a single dominant band as expected. In the presence of excess gold nanoparticles, this band is not observable in the DNA origami configuration with 1 or 2 gold binding sites. Importantly the band corresponding to DNA origami alone is still observable in the presence of excess gold nanoparticles when the DNA origami is configured to contain no binding sites (FIG. 14A). Combined, this suggests that gold nanoparticles bind specifically to DNA origami only when gold binding sites are present.

Gold nanoparticles alone produced a laddering of bands that are indicative of oligomerisation. However, no additional band was observed when gold nanoparticles were bound to DNA origami, thus making it difficult to determine the number of gold nanoparticles bound to DNA origami scaffolds (FIG. 14A)

To quantify the number of gold nanoparticles bound to DNA origami scaffolds and to confirm that they had bound to their specific binding sites, DNA origami-gold complexes were visualised with transmission electron microscopy. Examples of properly assembled particles observed in electron micrographs are shown in FIG. 14B, in which two gold nanoparticles were bound on either side of the DNA origami scaffold, consistent with design. In principle, this configuration of gold nanoparticles and DNA origami constitutes a functional plasmonic nanoantenna that will enhance the fluorescence intensity of a fluorescent molecule in the cavity of the DNA origami scaffold. Examples of hold nanoparticle monomers and aggregates in electron micrographs are shown in FIG. 14C. Some aggregates appeared to result from DNA origami-mediated daisy chaining, while other aggregates appeared to result from a direct interaction between gold nanoparticles.

### Assembly Yield of Plasmonic Nanoantenna Particles Assessed by Electron Microscopy

Transmission electron micrographs were used to quantify the proportion of gold nanoparticles bound to DNA origami structures and their configurations (monomer, dimer, aggregate), with different configurations of DNA origami scaffold (0, 1 or 2 binding sites) and with two different sulfhydryl DNA extensions (25-TG and 25-AC).

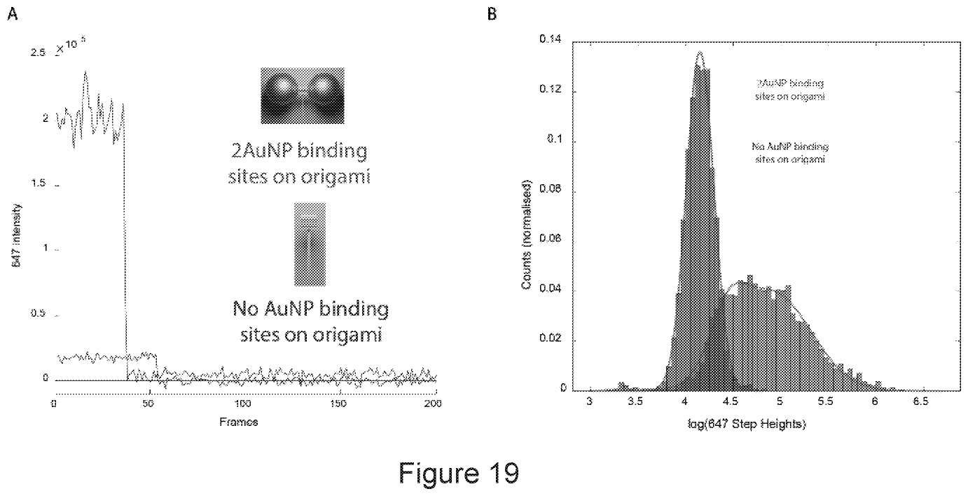

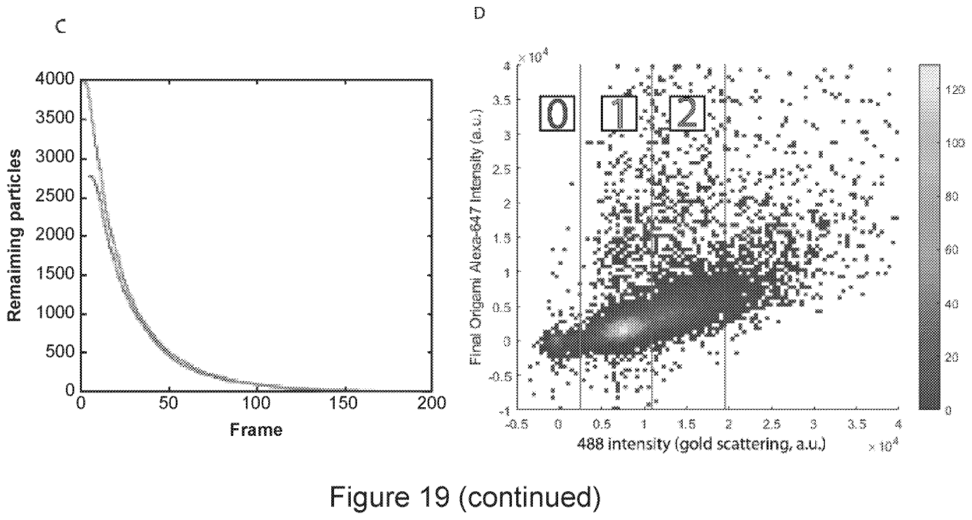

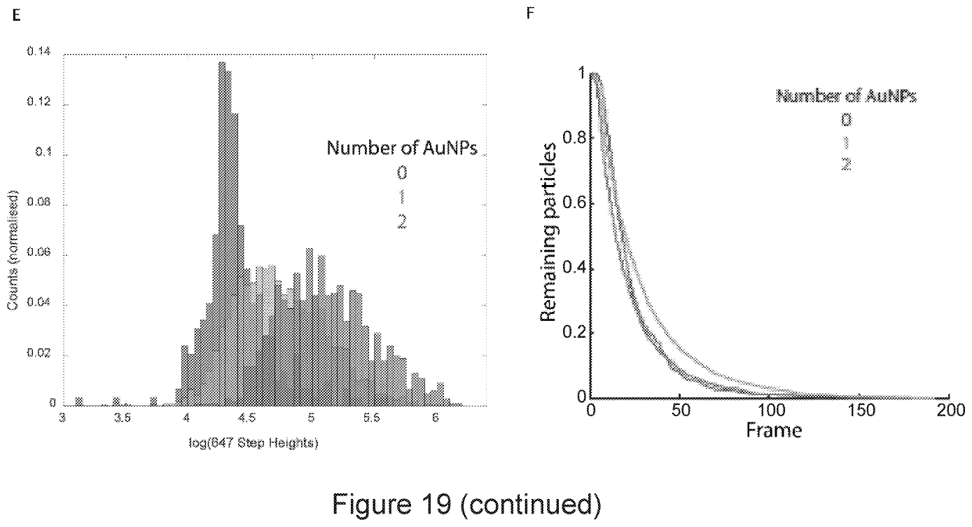

With two binding sites, 100% of DNA origami nanospanno structures were bound to AuNPs with no free DNA origami structures observed in electron micrographs. 22% of molecules were dimers, ~10% monomers and ~65% were aggregates or other poorly assembled structures (FIGS. 19 A and D). For DNA origami structures with 1 binding site, only half were bound to AuNPs. No dimeric assemblies were observed and 14-22% of AuNPs contained a single DNA origami structure bound (FIGS. 19 B and E). For DNA origami structures without gold binding sites, EM only yielded free gold and free NS with no evidence of assembled structures (FIGS. 19C and F). Combined these data indicate successful assembly of the specific, desired plasmonic nanoantenna architecture consisting of two 100 nm gold nanoparticles, on either side of a cavity within which DNA polymerase can be immobilised.

Co-Localisation of DNA Polymerase with DNA Origami Scaffold

In this section, we describe the specific attachment of polymerase-DNA to the cavity of the DNA origami scaffold. This occurs via the hybridisation of the DNA bound to polymerase enzymes to a complementary DNA staple extension located in the cavity (FIG. 16)

Measurement of Bulk Co-Localisation with Agarose Gel Electrophoresis

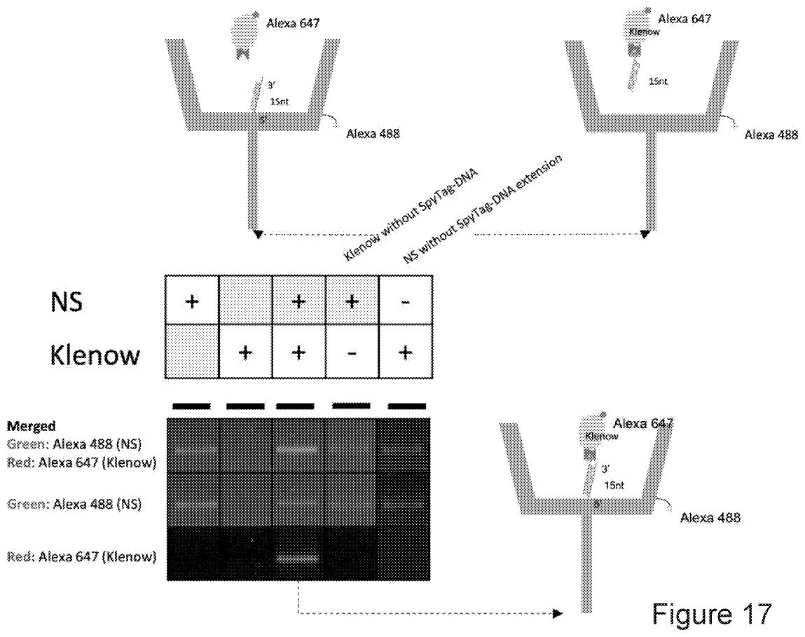

Binding was assessed in bulk by observing co-localisation of the DNA origami scaffold labelled with Alexa 488 fluorescent dye with DNA polymerase, which was labelled with Alexa 647 dye on agarose gel electrophoresis. We observe colocalization of DNA polymerase with DNA origami only when DNA polymerase was bound to SpyTag DNA. We also observe that colocalization resulted in a slower migration rate of both DNA origami alone and DNA polymerase alone (FIG. 17). These data binding of DNA polymerase by specific hybridisation to the DNA staple extension in the cavity of DNA origami.

Quantifying Co-Localisation Yield with Single Molecule Fluorescence Microscopy

To quantify co-localisation yield between DNA polymerase and DNA origami scaffolds, single-molecule fluorescence microscopy was used to determine the proportion of single DNA origami molecules that were bound to a single DNA polymerase enzyme. The presence of single DNA polymerase enzymes was determined by photobleaching where only molecules exhibiting a single photobleaching step were used for yield analysis (FIGS. 18A and B). In contrast, we found that the Alexa 488 fluorophores bound to DNA origami were resistant to photo bleaching. We therefore determined the presence of single DNA origami particles by the initial fluorescence intensity upon excitation with 488 nm light. By plotting the initial intensity distribution of all fluorescent particles, single DNA polymerases were readily discernible from oligomers or aggregates (FIG. 18C). By these measures we were able to determine the yield of single DNA origami scaffolds that were co-localised to single DNA polymerase molecules of around 90%, irrespective of the location of the DNA polymerase binding site (FIG. 18D). We note that as a positive control, we annealed an excess of Alexa 647 DNA that was complementary to the DNA polymerase binding sites. This provides a measure of the maximal possible occupancy of DNA polymerase, which was also 90% (FIG. 18D).

### Example 3. Characterisation of Plasmonic Nanoantenna

### Enhancement of Fluorescent Dyes Fluorescently Bound in Hotspot

To determine the function of the plasmonic nanoantenna, we experimentally quantified the intensity of fluorophores bound to the DNA origami scaffold with alone or with one or a pair of gold nanoparticles bound.

### Distribution of Intensities Measured by Single Molecule Photobleaching

The intensity of an immobilised Alexa 647 fluorophore within a plasmonic nanoantenna was measured by single molecule photobleaching assays. Alexa647-labelled DNA origami scaffolds with either zero or two AuNP binding sites were incubated at 100 pM with 500 pM AuNPs for 2 hours before being diluted 1 in 10 for TIRF microscopy measurements (see section of Example 1 headed “Single molecule total internal reflection fluorescence (TIRF) microscopy”). FIG. 19A provides an example of a single photobleaching step on a DNA origami scaffold with no AuNPs (blue), which is substantially less intense than the intensity of a single fluorophore in a DNA origami scaffold with a pair of AuNPs (red). FIG. 19B shows intensity distributions for ~3000 individual assemblies containing either no AuNP binding sites (blue) or two AuNP binding sites (red). These data reveal a broad distribution of fluorescence enhancement in the presence of two gold nanoparticles from 1 to 200-fold. This broad distribution is due to several factors including assembly yield of 2×AuNPs:1×DNA origami scaffold, the orientation of plasmonic nanoantennas on the coverslip, possible anisotropy in fluorophore labelling, variations in the distance between nanoparticle pairs and inhomogeneities in nanoparticle size and shape. The bleaching rates (FIG. 19C) were similar implying a higher total photon count from fluorophores in the presence of AuNPs.

TEM experiments revealed a yield of correctly assembled nanoantennas of 22%. Since scattering from gold nanoparticles is significant and their scattering cross section at 488 nm scales with the number of particles in a cluster, single DNA origami scaffolds with 0, 1, 2 or more gold nanoparticles were identified (FIG. 19D). Importantly, the proportion of dimerised gold nanoparticles was similar to that observed directly with electron microscopy (FIG. 18D and Table 4). The distributions of fluorophore intensities in DNA origami scaffolds with 0, 1 or 2 gold nanoparticles as measured by scattering were segregated. The mean intensity of fluorophores on DNA scaffolds with two gold nanoparticles was 10-fold greater than the intensity of fluorophores on DNA scaffolds with no gold nanoparticles (FIG. 19E). Notably the range of fluorescence enhancement varied substantially when two gold nanoparticles were present, with some particles showing no fluorescence enhancement, and others showing over 100-fold enhancement. This was not unexpected since the plasmonic enhancement effect relies on the orientation of pairs of gold nanoparticles to be in alignment with the polarisation of incident light. The rate of photobleaching was quantified and again little dependence on the number of gold nanoparticles bound was found (FIG. 19F).

TABLE 4
Quantification of populations of single Alexa647-
labelled origami bleach steps with corresponding
scattering intensities of 0, 1 or 2 AuNPs.

| | 0 AuNP | 1 AuNP | 2 AuNP |
|---|---|---|---|
| | binding sites | binding sites | binding sites |
| | (2628 particles) | (6963 particles) | (3121 particles) |
| 0 AuNPs | 99.5% | 45% | 12% |
| 1 AuNPs | 0.5% | 46% | 63% |
| 2 AuNPs | 0% | 12% | 25% |

### Fluorescence Intensity Enhancement as Measured by DNA PAINT

While photobleaching experiments are useful to determine assembly composition, stoichiometry and fluorescence intensities in static molecules, DNA sequencing requires repeated measurements of fluorescent dyes as they bind transiently within the hotspot of the plasmonic nanoantenna. To establish conditions required for DNA sequencing, the repeated transient binding of fluorescent DNA strands to 7 base DNA staple extensions in the hotspot of the plasmonic nanoantenna was measured. Repeated measurements on the same molecule also revealed whether a bright particle was always bright or whether there were significant fluctuations in the fluorescence intensity from a single plasmonic nanoantenna.

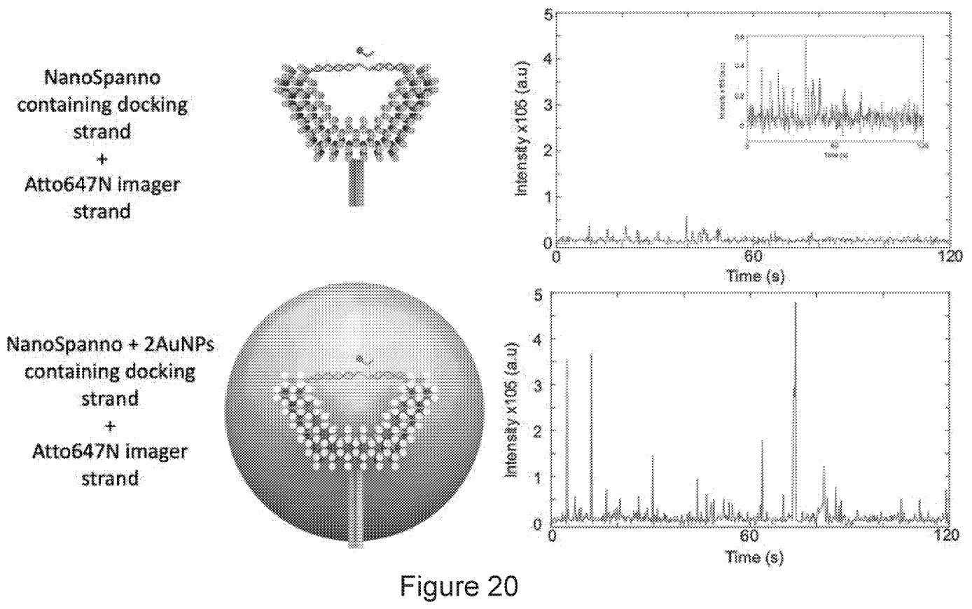

FIG. 20 shows fluorescence intensity spikes that occur as a result of the transient binding of a fluorescent DNA strand in the cavity of the DNA origami structure in the presence and absence of pairs of immobilised gold nanoparticles. A 10-50-fold fluorescence enhancement was observed in the presence of gold pairs, thus confirming that the plasmonic nanoantenna is functioning to enhance fluorescent signals. The large range in enhancement intensities again likely results from fluctuations in the orientation of gold nanoparticles within the duration of the experiment. A low number of intensity spikes in the absence of imager strands (FIG. 21) was also observed. The origin of these spikes is thought to be due to fluctuations in the orientation of the gold nanoparticles, which increases the scattering intensity and appears as a peak in the intensity trace, which could lead to false positive events in a sequencing measurement.

The effect of rotating the polarisation field relative to the orientation of gold nanoparticles is experimentally characterised below.

### Fluorescence Enhancement of DNA Polymerase in Hotspot

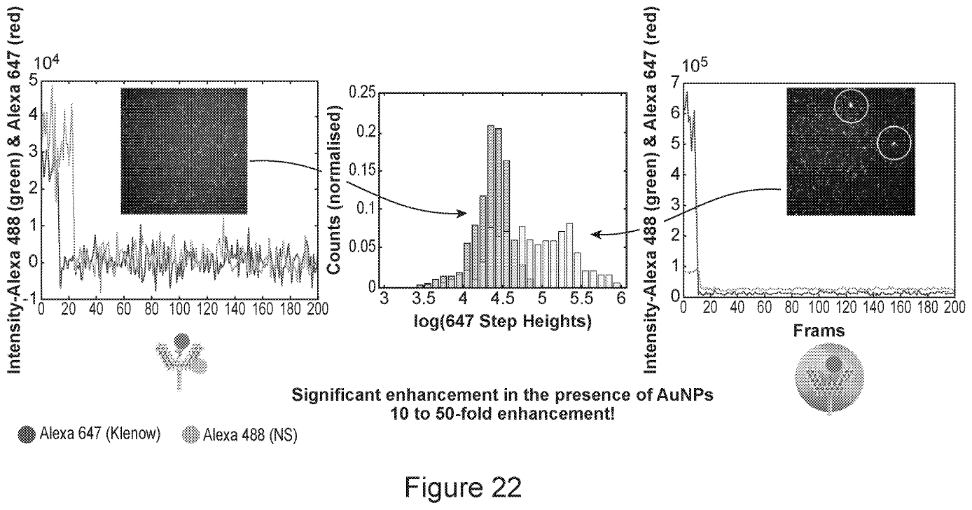

Next, to complete the synthesis of the plasmonic nanoantenna DNA sequencer a fluorescently labelled DNA polymerase molecule was bound in the hotspot of the plasmonic nanoantenna. The intensity of single fluorescent DNA polymerase enzymes bound to the cavity of the DNA origami scaffold without gold and with a pair of gold nanoparticles was measured with single molecule photobleaching measurements. Example bleach traces without and with gold are on the left and right of FIG. 22 respectively and demonstrate two orders of magnitude increase in fluorescent signal in the presence of gold pairs. As above the full distribution of plasmonic enhancement with pairs of gold nanoparticles is broad as expected since the gold spheres were free to rotate. Nonetheless, these data provide direct evidence that DNA polymerase has been successfully immobilised in the hotspot of a plasmonic nanoantenna. This is the first example of a fluorescently enhanced protein molecule by a plasmonic nanoantenna.

### Characterising Fluorescence Enhancement at Different Polarisation Angles

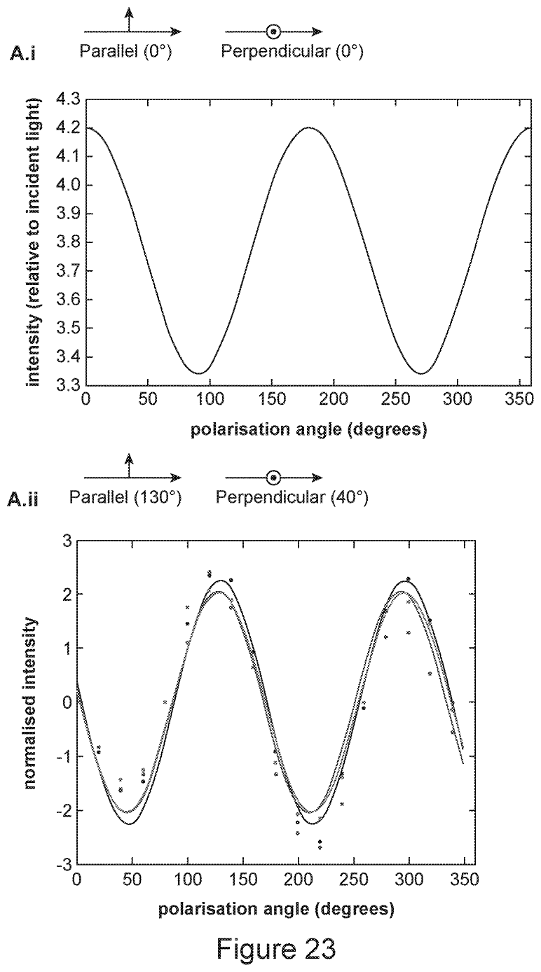

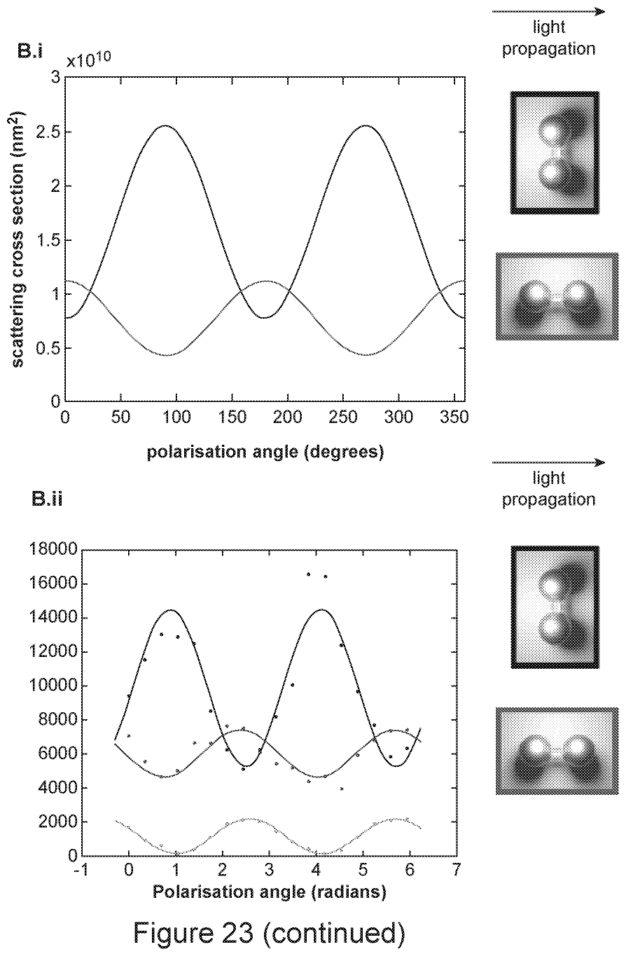

In order to determine the effect of the polarisation of incident light on the scattering intensity of plasmonic nanoantennas, simulations of the scattering cross-section, light intensity and fluorescence enhancement were performed at varying incident polarisation angles for nanoantennas in perpendicular orientations on a glass surface. FIG. 23Ai shows how the TIR intensity above the glass coverslip varies between fully p-polarised (parallel, 0°) where the polarisation is normal to the glass coverslip and fully s-polarised (perpendicular, 90°) where the polarisation is along the glass coverslip. This was used (FIG. 23Aii) to experimentally calibrate the polarisation angle based on measured background intensity as incident polarisation is being varied by a half-waveplate (see section in Example 1 headed “Measurement of gold scattering intensity vs polarisation angle”). FIG. 23Bi shows the simulated scattering cross-section of a nanoantenna oriented either in the direction of propagation of the TIRF field (blue) or perpendicular to the propagation (black). In the blue orientation, there is no optimal alignment of the nanoantenna with the incident polarisation, so the scattering cross-section across all polarisation angles is on average lower, and the variation with polarisation angle follows that of the TIR intensity. In the black orientation, the variation of the scattering cross section is 90° out of phase and higher on average since s-polarised light aligns with the nanoantenna in this orientation. FIG. 27Bii shows the experimentally measured intensity of scattered light for various particles as the polarisation angle is varied, enabling the measurement of nanoantenna aligned perpendicular to the propagation of the TIRF field (black), parallel (blue), or single AuNPs (green).

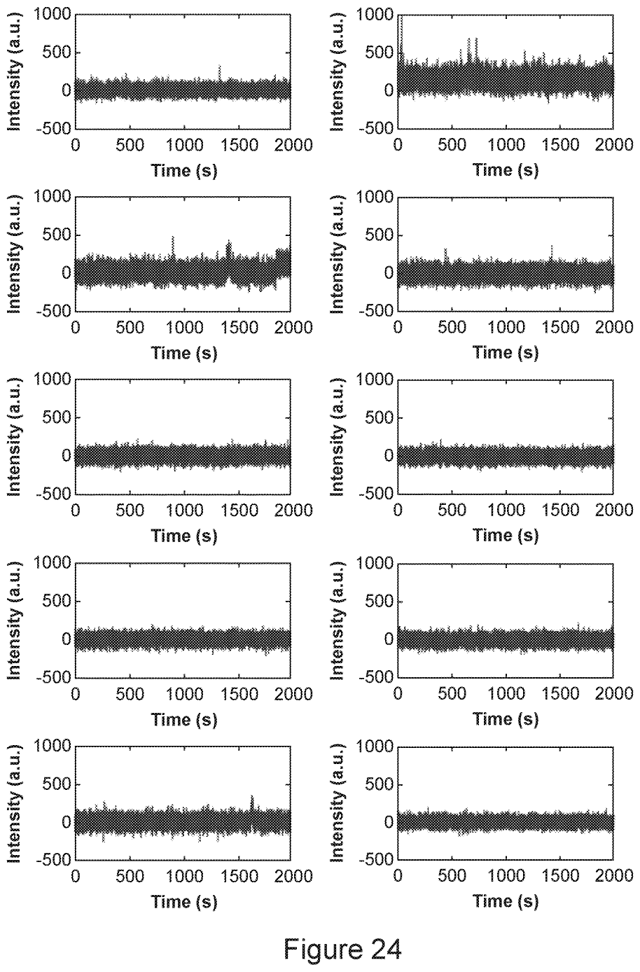

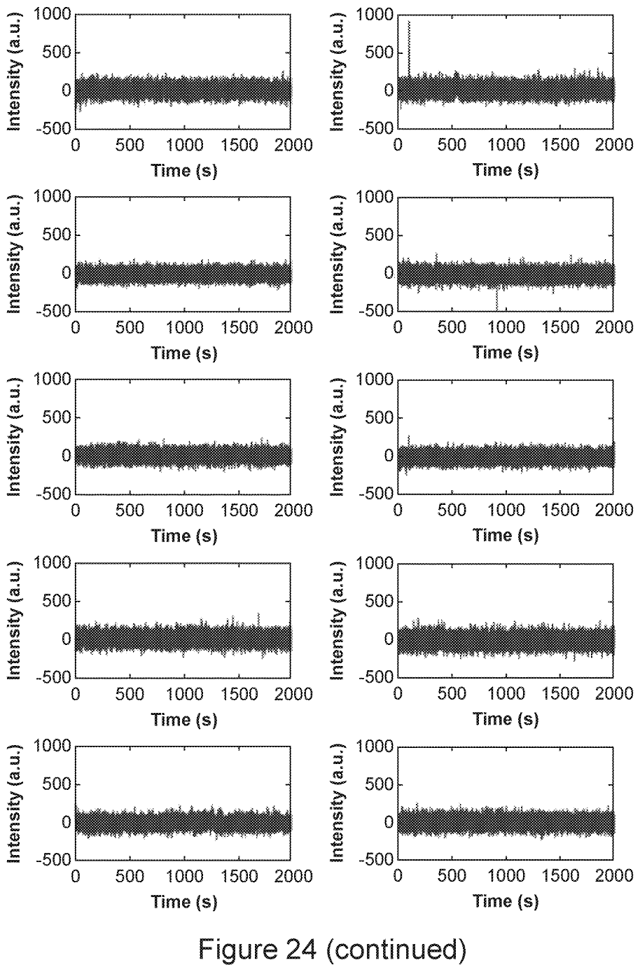

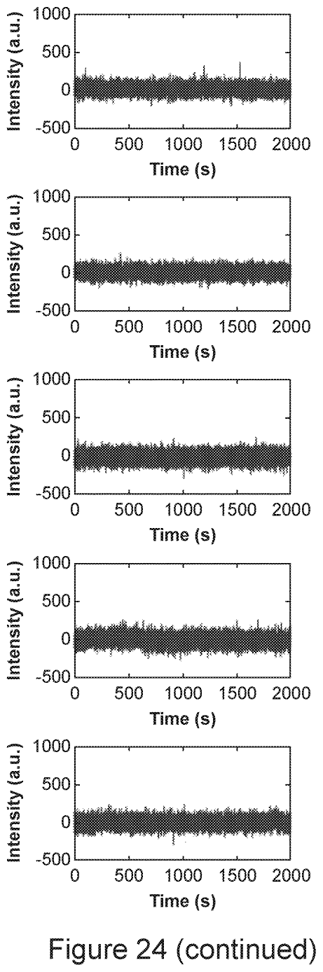

When the TIRF field is s-polarised there are significant changes in measured intensity based on the orientation of the nanoantenna. In order to prevent this from occurring within the duration of a sequencing experiment, the AuNPs were immobilised on the glass coverslip through a biotin:streptavidin interaction. This was achieved by flowing biotinylated DNA complementary to the DNA bound to AuNPs after their initial attachment to the functionalised coverslip via the biotinylated DNA origami scaffold. Measurements of particles for ~30 mins following this step show a greatly reduced number of intensity spikes (FIG. 24).

The effect of the orientation of the nanoantenna on the enhancement of an Alexa-647 fluorophore was also simulated (FIG. 25), showing a variation in enhancement to below 1 to 200-fold. The distribution of enhancement for a randomly oriented population of nanoantennas is similar to that seen above.

### Example 4. Single-Molecule DNA Sequencing Reactions

Fluorophore Labelled dNTP

DNA sequencing was achieved by detecting the incorporation of single fluorescent nucleotides by the DNA polymerase enzyme located in the plasmonic hotspot, with each nucleotide (ATP, GTP, TTP or CTP) labelled with a different colour dye. Importantly, fluorescent probes need to be attached to the terminal phosphate of nucleotides, which prevents termination of the polymerase reaction and means that upon nucleotide incorporation the fluorescent probe is released upon phosphodiester bond formation. Nucleotides with 6-aminohexyl groups attached to the distal phosphate were purchased from Jena Biosciences, allowing attachment of succinimidyl fluorophores (Sigma) using NHS ester reaction chemistry methods prior to purification with HPLC.

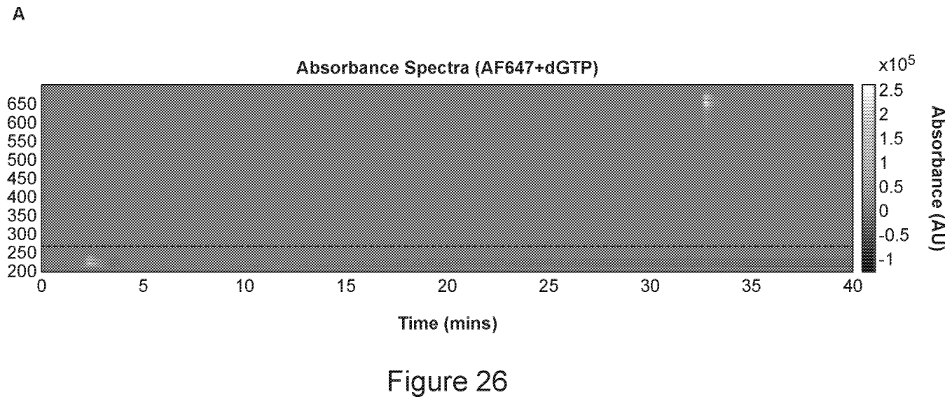

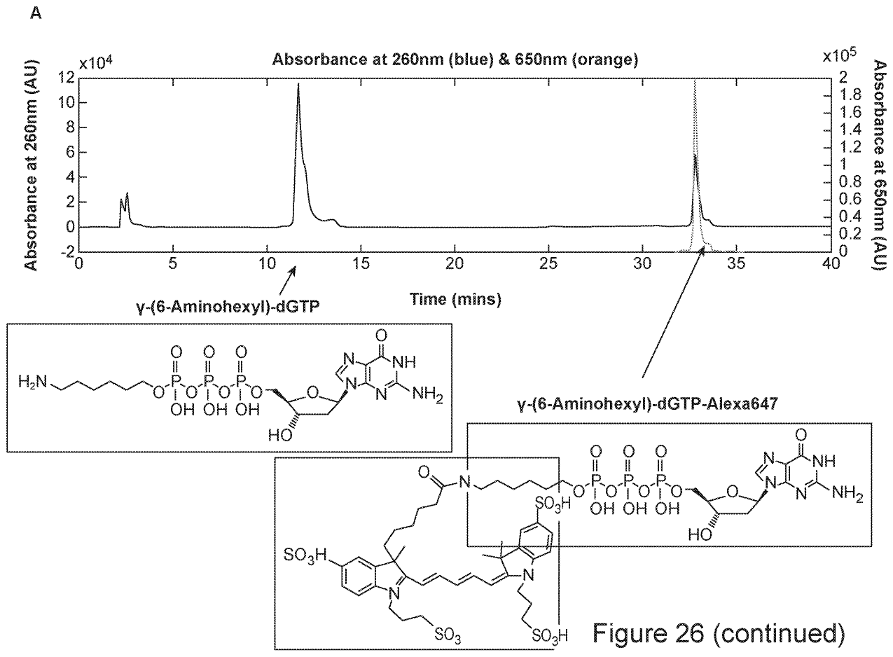

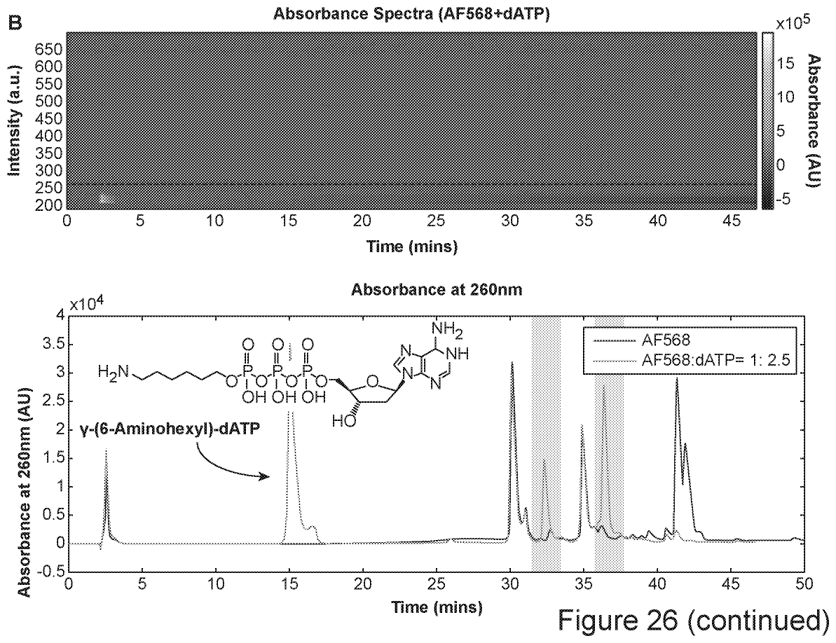

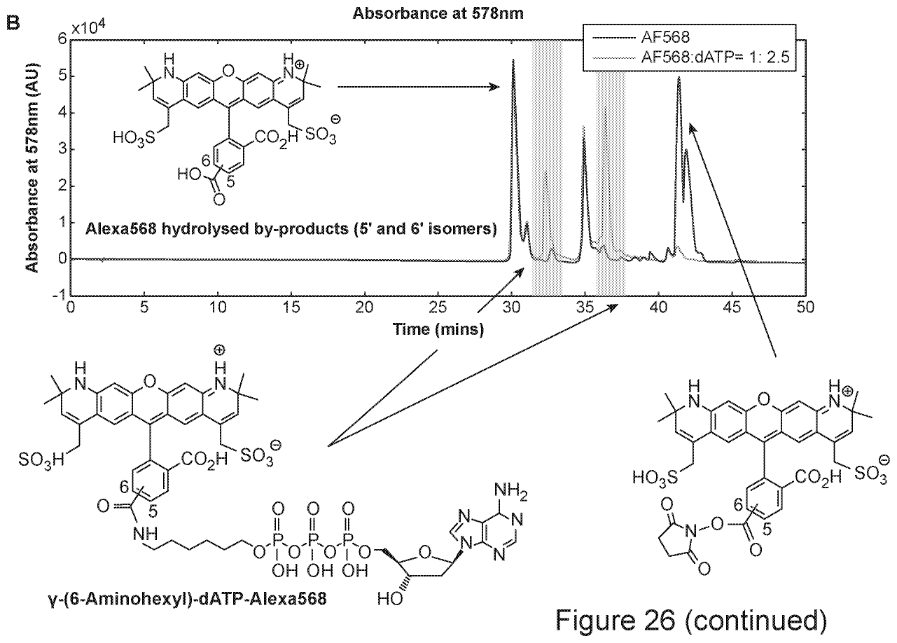

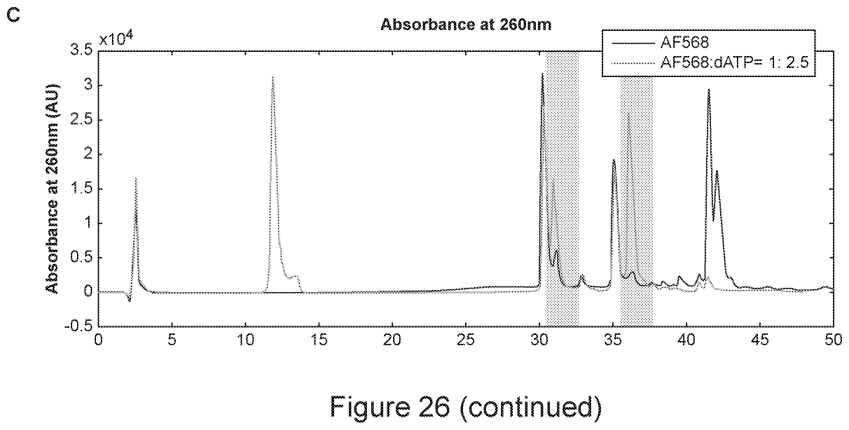

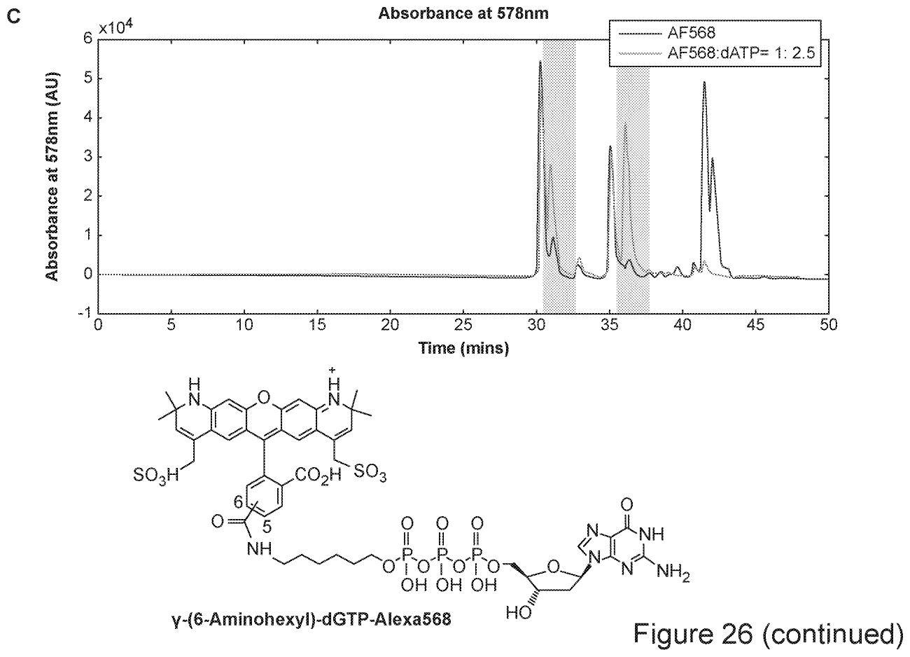

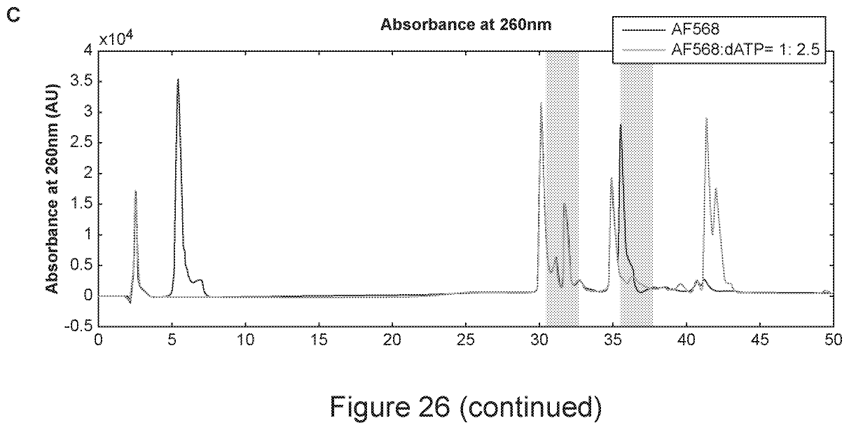

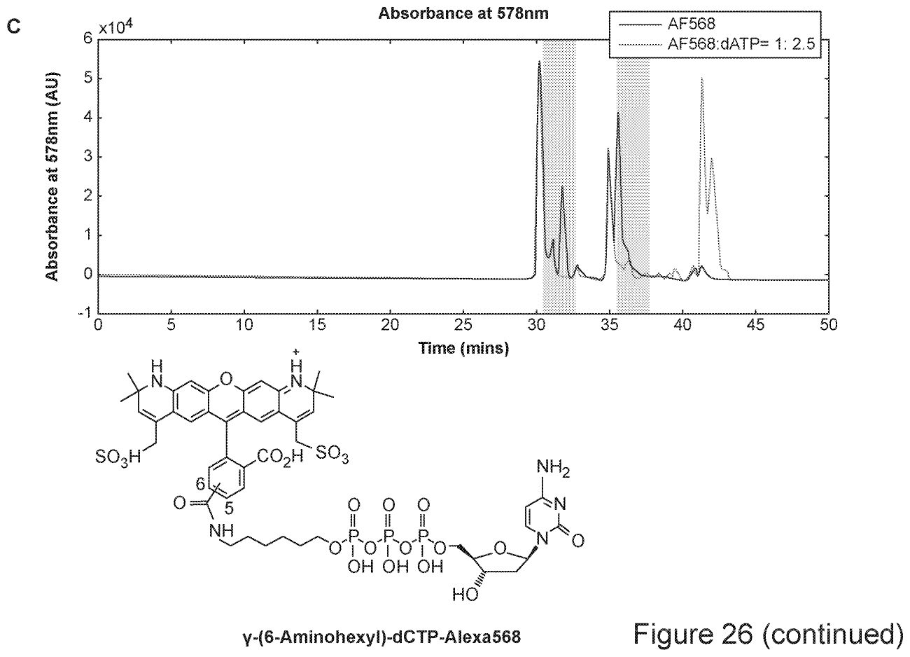

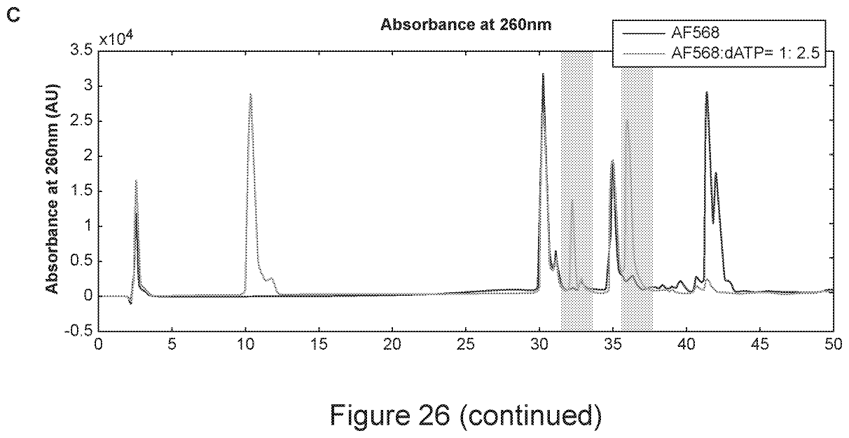

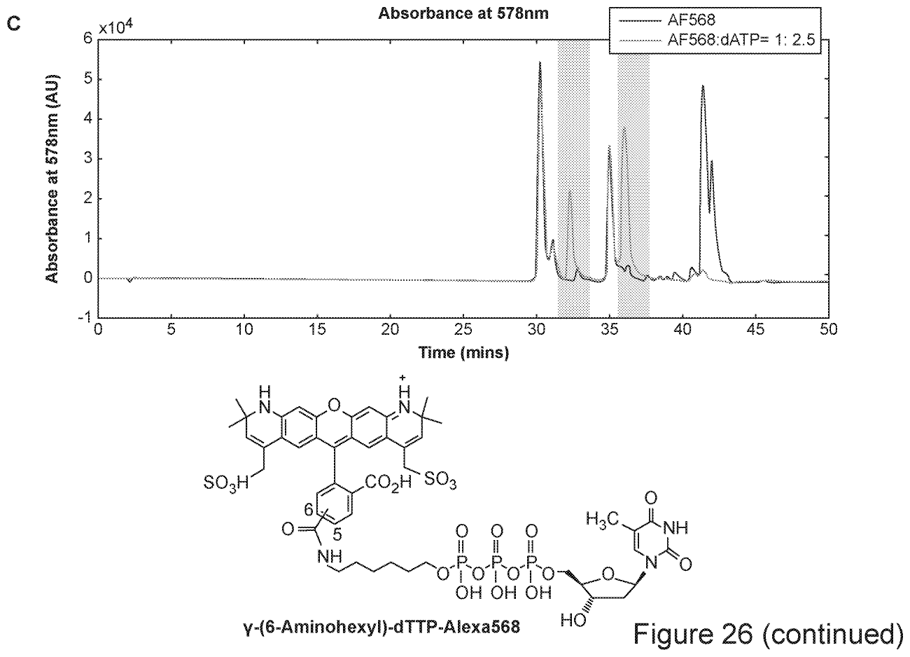

Aminated_dGTP normally elutes at around 12 min whereas Alexa647 labeled dGTP will normally elute at around 32 min (FIG. 26A). The fractions containing pure product were evaporated, followed by co-evaporation with methanol:MQW=1:1 (2×). The residue was dissolved in water and was quantitated by UV-Vis spectrophotometry then split into 500 pmol/tube (so that resuspending in 10 uL MQW gives 50 uM working stock), dried and stored at −20° C.

Alexa568 has two isomers that typically elute at approximately 41 and 42 minutes, appearing as a blue color at both 260 nm and 578 nm (FIG. 26B). These isomers have a hydrolyzed by-product that elutes at 30 and 35 minutes. Aminated_dGTP typically elutes at around 12 minutes, appearing as an orange color at 260 nm, while Alexa568 labeled dGTP generally elutes at around 31 and 36 minutes due to its two isomers, appearing as an orange color at both 260 nm and 578 nm (FIG. 26B). Aminated_dATP typically elutes at around 15 minutes, and Alexa568 labeled dTTP typically elutes at around 32 and 36 minutes. Aminated_dTTP typically elutes at around 10 minutes, and Alexa568 labeled dTTP generally elutes at around 32 and 36 minutes. Finally, Aminated_dCTP usually elutes at around 5 minutes, while Alexa568 labeled dCTP typically elutes at around 32 and 36 minutes (FIG. 26C).

### Configuring the Plasmonic Nanoantenna for DNA Sequencing

For initial proof of concept DNA sequencing experiments, the plasmonic nanoantenna was configured to contain both a single DNA polymerase enzyme immobilised with a 71-base single-stranded DNA linker and the primed target DNA strand (FIG. 27). This configuration removed the requirement to capture freely diffusing DNA. Thus, minimising the parameters in the experiment. The length of DNA reads will also be sterically limited.

2-Colour DNA Sequencing Experiments with Alexa647 Labelled dGTP and Alexa568 Labelled dATP

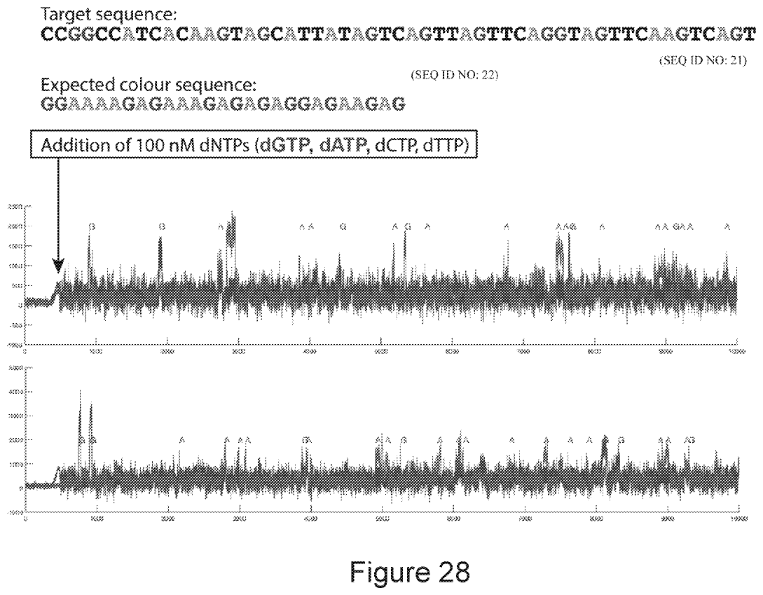

As a proof of concept, the plasmonic nanoantenna DNA sequencer was assembled in the configuration illustrated in FIG. 27, which were immobilised on the surface of a glass coverslip for imaging with TIRF microscopy. To identify fully assembled plasmonic nanoantenna sequencers, single molecule photobleaching assays were performed and scattering intensities measured to determine particles that had exactly one DNA origami scaffold, one DNA polymerase enzyme and a pair of gold nanoparticles. Fluorescence intensity around these fully assembled particles were monitored over time with simultaneous excitation with 568 and 647 lasers, to yield a 2-colour intensity trace. This led to a substantial increase in fluorescence background as expected. In addition, distinct fluorescent spikes were observed that were well above background (FIG. 28). Base calling was determined simply as any intensity spike that was above 3 standard deviations above background. This base calling algorithm led to multiple plasmonic nanoantenna DNA sequencers identifying bases in exactly the correct sequence for read lengths of 8-16 bases. Two example traces are shown in FIG. 28. Notably no correct sequences were identified in control experiments that lacked a single critical component such as DNA polymerase or the target DNA template strand.

1.-106. (canceled)

107. A system comprising:
a) two plasmonic particles;
b) a nucleic acid scaffold coupled to the two plasmonic particles; and
c) a protein, wherein the protein is bound to the nucleic acid scaffold.

108. The system of claim 107, further comprising a nucleic acid analyte bound to the nucleic acid scaffold.

109. The system of claim 108, wherein the nucleic acid analyte is bound non-covalently to the nucleic acid scaffold.

110. The system of claim 107, wherein the two plasmonic particles are bound to the nucleic acid scaffold and positioned relative to one another such that a plasmonic hotspot exists between the two plasmonic particles; and wherein the plasmonic hotspot comprises a region unoccupied by the nucleic acid scaffold.

111. The system of claim 110, wherein the region unoccupied by the nucleic acid scaffold is defined by a three-dimensional space having a length (L) between the two plasmonic nanoparticles, a height (H) at a midpoint (L/2) that is perpendicular to (L), and a width (W) at L/2 that is perpendicular to L and H, wherein W is selected from 20 nanometers (nm) to about 100 nm, wherein H is selected from 10 nm to about 100 nm, and wherein L is measured at the shortest distance between the two plasmonic nanoparticles.

112. The system of claim 111, wherein L is selected from 20 nm to 100 nm.

113. The system of claim 110, wherein the region unoccupied by the nucleic acid scaffold has a volume of at least about 1 zeptoliter (zL).

114. The system of claim 110, wherein the region unoccupied by the nucleic acid scaffold is 80% or more of the plasmonic hotspot.

115. The system of claim 107, wherein each of the two plasmonic particles is metallic.

116. The system of claim 115, wherein a plasmonic particle of the two plasmonic particles comprises gold, silver, aluminum, copper, palladium, platinum, or an alloy thereof.

117. The system of claim 107, wherein each of the two plasmonic particles has a largest diameter of about 5 nm to about 500 nm.

118. The system of claim 107, wherein each of the two plasmonic particles are coated with a coating.

119. The system of claim 118, wherein the coating reduces aggregation of the two plasmonic particles by 50% or more relative to the two plasmonic particles lacking the coating.

120. The system of claim 118, wherein the coating comprises a plurality of oligonucleotides.

121. The system of claim 120, wherein the two plasmonic particles are anchored to the nucleic acid scaffold by nucleic acid linkers formed by an oligonucleotide of the plurality of oligonucleotides and a polynucleotide forming part of, or extending from, the nucleic acid scaffold.

122. The system of claim 107, wherein the nucleic acid scaffold comprises DNA origami structures, and wherein at least a portion of the DNA origami structures are assembled to form a U-shaped or curved structure.

123. The system of claim 107, wherein the protein is a nucleic acid polymerase.

124. A method comprising:
a) providing:
i) two plasmonic particles;
ii) a nucleic acid scaffold coupled to the two plasmonic particles; and
iii) a protein, wherein the protein is bound to the nucleic acid scaffold; and
b) with aid of the two plasmonic particles, generating an electrical field at the protein bound to the nucleic acid scaffold.

125. The method of claim 124, wherein (a) further comprises contacting the protein with the nucleic acid analyte and a labelled nucleotide.

126. The method of claim 124, further comprising determining a sequence of the nucleic acid analyte.


SEQUENCE LISTING (28 sequences)

SEQ ID NO: 1 - DNA, 15 bp, synthetic construct
  source 1..15: mol_type = other DNA
aaaaaaaaaaaaaaa

SEQ ID NO: 2 - DNA, 15 bp, synthetic construct
  source 1..15: mol_type = other DNA
ttttttttttttttt

SEQ ID NO: 3 - DNA, 15 bp, synthetic construct
  source 1..15: mol_type = other DNA
agagagagagagaga

SEQ ID NO: 4 - DNA, 15 bp, synthetic construct
  source 1..15: mol_type = other DNA
tctctctctctctct

SEQ ID NO: 5 - DNA, 25 bp, synthetic construct
  source 1..25: mol_type = other DNA
agagagagagagagagagagagaga

SEQ ID NO: 6 - DNA, 25 bp, synthetic construct
  source 1..25: mol_type = other DNA
tctctctctctctctctctctctct

SEQ ID NO: 7 - DNA, 25 bp, synthetic construct
  source 1..25: mol_type = other DNA
acacacacacacacacacacacaca

SEQ ID NO: 8 - DNA, 25 bp, synthetic construct
  source 1..25: mol_type = other DNA
tgtgtgtgtgtgtgtgtgtgtgtgt

SEQ ID NO: 9 - DNA, 25 bp, synthetic construct
  source 1..25: mol_type = other DNA
tgtgtgtgtgtgtgtgtgtgtgtgt

SEQ ID NO: 10 - DNA, 25 bp, synthetic construct
  source 1..25: mol_type = other DNA
acacacacacacacacacacacaca

SEQ ID NO: 11 - DNA, 25 bp, synthetic construct
  source 1..25: mol_type = other DNA
ttttttttttttttttttttttttt

SEQ ID NO: 12 - DNA, 25 bp, synthetic construct
  source 1..25: mol_type = other DNA
aaaaaaaaaaaaaaaaaaaaaaaaa

SEQ ID NO: 13 - DNA, 25 bp, synthetic construct
  source 1..25: mol_type = other DNA
acacacacacacacacacacacaca

SEQ ID NO: 14 - DNA, 25 bp, synthetic construct
  source 1..25: mol_type = other DNA
tgtgtgtgtgtgtgtgtgtgtgtgt

SEQ ID NO: 15 - DNA, 25 bp, synthetic construct
  source 1..25: mol_type = other DNA
aaaaaaaaaaaaaaaaaaaaaaaaa

SEQ ID NO: 16 - DNA, 25 bp, synthetic construct
  source 1..25: mol_type = other DNA
ttttttttttttttttttttttttt

SEQ ID NO: 17 - DNA, 64 bp, synthetic construct
  source 1..64: mol_type = other DNA
gaaggagatataccatgggcagcagccatcaccatcatcaccacagccaggatccgaattcgag

SEQ ID NO: 18 - DNA, 15 bp, synthetic construct
  source 1..15: mol_type = other DNA
cactcggcaggacgg

SEQ ID NO: 19 - DNA, 15 bp, synthetic construct
  source 1..15: mol_type = other DNA
cgagccgctgactgg

SEQ ID NO: 20 - DNA, 53 bp, synthetic construct
  source 1..53: mol_type = other DNA
ggccggtagtgttcatcgtaatatcagtcaatcaagtccatcaagttcagtca

SEQ ID NO: 21 - DNA, 53 bp, synthetic construct
  source 1..53: mol_type = other DNA
ccggccatcacaagtagcattatagtcagttagttcaggtagttcaagtcagt

SEQ ID NO: 22 - DNA, 27 bp, synthetic construct
  source 1..27: mol_type = other DNA
ggaaaagagaaagagagaggagaagag

SEQ ID NO: 23 - DNA, 15 bp, synthetic construct
  source 1..15: mol_type = other DNA
  modified_base 1: mod_base = OTHER; note = 4-(N-Maleimidomethyl)cyclohexanecarboxylic acid N-hydroxysuccinimide ester modified nucleotide
cactcggcaggacgg

SEQ ID NO: 24 - DNA, 15 bp, synthetic construct
  source 1..15: mol_type = other DNA
  modified_base 15: mod_base = OTHER; note = 4-(N-Maleimidomethyl)cyclohexanecarboxylic acid N-hydroxysuccinimide ester modified nucleotide
cgagccgctgactgg

SEQ ID NO: 25 - DNA, 15 bp, synthetic construct
  source 1..15: mol_type = other DNA
cgagccgctgactgg

SEQ ID NO: 26 - DNA, 15 bp, synthetic construct
  source 1..15: mol_type = other DNA
  modified_base 15: mod_base = OTHER; note = 4-(N-Maleimidomethyl)cyclohexanecarboxylic acid N-hydroxysuccinimide ester modified nucleotide
cgagccgctgactgg

SEQ ID NO: 27 - protein, 6 aa, synthetic construct
HHHHHH

SEQ ID NO: 28 - protein, 17 aa, synthetic construct
MGSSHHHHHHSQDPNSS